(19) United States
(12) Patent Application Publication
(10) Pub. No.: US 2009/0012045 A1
(43) Pub. Date: Jan. 8, 2009

(54) Methods of Treating Cell Proliferative Disorders

(75) Inventors: Yasumichi Hitoshi, Brisbane, CA (US); Elliott Grossbard, San Francisco, CA (US); Ankush Argade, Foster City, CA (US); Rajinder Singh, Belmont, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: RIGEL PHARMACEUTICALS, INC., South San Francisco, CA (US)

(21) Appl. No.: 12/147,387

(22) Filed: Jun. 26, 2008

Related U.S. Application Data
(60) Provisional application No. 60/946,248, filed on Jun. 26, 2007.
(60) Provisional application No. 61/016,203, filed on Dec. 21, 2007.

(51) Int. Cl. A61K 31/538; A61P 35/00; A61K 31/5383; C12N 5/00; A61K 31/675; A61K 31/5415

(52) U.S. Cl. 514/81; 514/230.5; 514/224.2; 435/375

(57) ABSTRACT

The present disclosure provides methods for the treatment of cell proliferative disorders by administration of a RET kinase inhibitor. Cell proliferative disorders treatable by the methods include, thyroid tumors.

### CROSS REFERENCE TO RELATED APPLICATIONS

This application claims the benefit of the filing dates, under 35 USC §119(e), of U.S. Provisional Application Ser. No. 60/946,248, filed 26 Jun. 2007; and U.S. Provisional Application Ser. No. 61/016,203, filed 21 Dec. 2007, each of which are hereby incorporated by reference in their entirety.

### BACKGROUND OF THE INVENTION

1. Field of the Invention

The present disclosure relates to methods and compositions for treating cell proliferative disorders, where the compositions comprise inhibitors that target kinase activities affecting the proliferative potential of cells. In particular, the present disclosure concerns methods of inhibiting proliferation of tumor cells and treating solid tumor cancers using certain 2,4-pyrimidinediamine compounds or prodrugs thereof.

2. Summary of the Related Art

Cancer is a group of varied diseases characterized by uncontrolled, abnormal growth and division of cells. Cancer cells typically bear one or more abnormalities in the molecular mechanisms that control of cell growth and division, such as cell cycle checkpoint controls or signaling pathways involved in cellular communication. Through successive rounds of mutation and natural selection, a group of abnormal cells, generally originating from a single mutant cell, accumulates additional mutations that provide selective growth advantage over other cells, and thus evolves into a cell type that predominates in the cell mass and continues to divide unchecked.

The process of mutation and natural selection is enhanced by genetic instability displayed by many types of cancer cells, an instability which is gained either from somatic mutations or by inheritance from the germ line. The enhanced mutability of cancerous cells increases the probability of their progression towards formation of malignant cells. As the cancer cells further evolve, cells from the resulting cell mass, or tumor, may become locally invasive and may spread through the blood or lymph to start new cancers in tissues other than the cancer cell's tissue of origin (metastases), colonizing and destroying surrounding normal tissues. This property along with the heterogeneity of the tumor cell population makes cancer a particularly difficult disease to treat and eradicate.

The cellular processes controlling cell division and cell proliferation are complex, involving an intricate interplay between gene products that promote cell division and growth and those that hold such processes in check. Positive regulators of growth and proliferation are generally described as proto-oncogenes, which are the normal counterparts of altered genes and their gene products known to promote tumor and cancer formation. Proto-oncogenes promote cell division and negatively control cell apoptosis. Uncoupling the activity of these gene products from their normal regulated state converts the proto-oncogenes to oncogenes. Normal function of proto-oncogenes includes growth factors, growth factor receptors, cellular signal transduction molecules, and nuclear factors. Activation of the proto-oncogenes into oncogenic forms can occur in a variety of ways, including gene mutation, amplification, gene translocation, and viral activation.

Tumor suppressors, as opposed to the proto-oncogenes, generally exert a negative effect on cell growth, promote apoptosis of cells, inhibit cell cycle progression, and affect invasive and metastatic potential. In some instances, tumor suppressors can counter the activity of oncogenes even in their altered forms. Upon loss or inhibition of tumor suppressor function, the unregulated activity of proto-oncogenes or their corresponding oncogenic forms leads to cell transformation and carcinogenesis. Gene mutation or deletion, suppressed transcription, increased degradation, or abnormalities of associated proteins that work in concert with the tumor suppressors may compromise tumor suppressor activity. Tumor suppressor genes act as recessive alleles such that a cell with a normal allele along with a mutant allele still behaves normally. Thus, loss of the normal allele, also called loss of heterozygozity (LOH), characterizes some types of abnormal cell growth and proliferation. Genomic instability arising as a consequence of oncogene activity and disruption of normal cell division controls can increase the probability of LOH and thus the occurrence of the transformed phenotype by oncogenes.

Traditional cancer treatments take advantage of the higher proliferative capacity of cancer cells and their increased sensitivity to DNA damage. Ionizing radiation, including γ-rays and x-rays, and cytotoxic agents, such as bleomycin, cisplatin, vinblastine, cyclophosphamide, 5′-fluorouracil, and methotrexate rely upon a generalized damage to DNA or block DNA synthesis mechanisms, destabilizing chromosomal structure and eventually leading to destruction of cancer cells. These treatments are particularly effective for those types of cancers that have defects in the cell cycle checkpoint, because such defects limit the ability of these cells to repair damaged DNA, or to properly replicate DNA before undergoing cell division. The non-selective nature of these treatments, however, often results in severe and debilitating side effects. The systemic use of these drugs may result in damage to normally healthy organs and tissues, and compromise the long term health of the patient.

Although more selective chemotherapeutic treatments have been developed based on knowledge of how cancer cells develop, for example, the anti-estrogen compound tamoxifen, the effectiveness of all chemotherapeutic treatments is subject to development of resistance to the drugs. In particular, the increased expression of cell membrane bound transporters, such as MdrI, produces a multidrug resistance phenotype characterized by increased efflux of drugs from the cell. These types of adaptation by cancer cells severely limit the effectiveness of certain classes of chemotherapeutic agents.

Treatment of cell proliferative disorders can also target the oncogenes and/or the tumor suppressors affected in the transformed cells. However, a disorder arising from a loss-of-function, such as a tumor suppressor, is typically more problematic when attempting to treat the underlying molecular defect than treating the underlying molecular defect in a disorder arising from a gain-of-function change, such as activation of an oncogene. Altering cellular processes to provide the lost cellular function is not practicable in many cases. Thus, even for cell proliferative disorders arising from loss of tumor suppressor activity, therapy is typically directed at the dysregulated molecules (e.g., proto-oncogenes) that act as a consequence of the lost tumor suppressor function. Although many molecular targets have been identified, such as non-receptor and receptor based protein kinases, the complex nature of the cellular regulatory mechanisms at play in cell proliferation and growth would indicate that other molecules that could be targets of therapy remain to be identified. Some of these will be unknown while others may be known but not linked to cell proliferative disorders.

Renal cell carcinoma is the sixth leading cause of cancer death, and is characterized by a lack of early warning signs, diverse clinical manifestations, resistance to radiation and chemotherapy, and infrequent but reproducible responses to immunotherapy agents such as interferon alpha and interleukin (IL)-2. Consequently, identification of other chemotherapeutic agents is critical for establishing therapies effective for attacking the heterogeneous nature of proliferative diseases such as cancer and for overcoming any resistance that may develop over the course of therapy with other compounds. Moreover, use of combinations of chemotherapeutic agents with differing properties and cellular targets increases the effectiveness of chemotherapy and limits the generation of drug resistance.

A related gastroenteropancratic neuroendocrin proliferative disorder with few treatment options is carcinoid, slow-growing but malignant tumour type, originating in the cells of the neuroendocrine system. Carcinoid tumours are apudomas that arise from the enterochromaffin cells throughout the gut. They are most commonly found in the foregut, lung, bronchus and trachea from where they rarely metastasise. In cases of metastases it can lead to carcinoid syndrome, due to the production of serotonin, which is released into the systemic circulation, leading to symptoms of cutaneous flushing, diarrhea, bronchoconstriction and right-sided cardiac valve disease. Currently, surgery to remove a carcinoid is the only curative therapy, with current chemotherapy options offering little benefit.

Thus it is desirable to identify cellular molecules that act in an oncogenic manner in cell proliferative disorders, either as a consequence of alteration of its own activity or as a result of loss of a cellular function that act to regulate its activity. Upon identification of such molecules, compounds specifically directed to that cellular molecule can be identified and used, either independently or in combination with known therapies, to treat the cell proliferative disorder.

### SUMMARY OF THE INVENTION

It has been discovered that certain 2,4-pyrimidinediamine compounds are potent inhibitors of proliferation of abnormal cells, such as tumor cells, in in vitro assays. In particular, these compounds have demonstrated potent inhibition against renal tumor cell lines. Further, these compounds have demonstrated potent inhibition against thyroid tumor cell lines. The compounds can therefore be used to inhibit proliferation of tumor cells in vitro and in vivo, in a variety of contexts. Prodrugs of the compounds that yield the active drug compound under the conditions of use can also be use to inhibit tumor cell proliferation in a variety of in vitro and in vivo contexts.

The present disclosure provides method of treating cell proliferative disorders by administration to subjects an amount of a RET kinase inhibitory compound effective to treat the cell proliferative disorder. In some embodiments, the RET kinase inhibitor is selective for RET kinase, thereby specifically targeting the aberrant RET kinase activity present in the proliferative disorder. Any cell proliferative disorder wherein RET plays a role in some aspect of abnormal cell division or cell growth can be treated with the inhibitor compounds. Some examples are MEN Type 2, thyroid carcinoma and pheochromocytoma.

In a first aspect, the invention provides methods for treating a disease or condition caused by a mutation in RET kinase, comprising administering to a subject in need of such treatment an amount of a compound according to formula (XII),

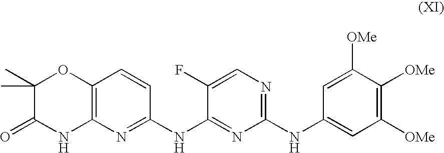

including salts, hydrates, solvates and N-oxides thereof.

In a second aspect, the invention provides methods for treating a disease or condition caused by a mutation in RET kinase, comprising administering to a subject in need of such treatment an amount of a prodrug compound according to formula (XII),

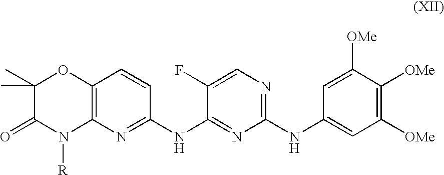

including salts, hydrates, solvates and/or N-oxides thereof, wherein R represents a progroup, as described below.

In a third aspect, the invention provides methods for treating a disease or condition caused by a mutation in RET kinase, comprising administering to a subject in need of such treatment an amount of a compound according to formula (XIII),

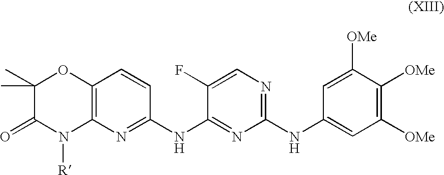

including salts, hydrates, solvates and/or N-oxides thereof, wherein R′ is hydrogen or a progroup, as described below.

In a fourth aspect, the invention provides methods for treating a disease or condition caused by a mutation in RET kinase, comprising administering to a subject in need of such treatment an amount of a compound according to formula (I),

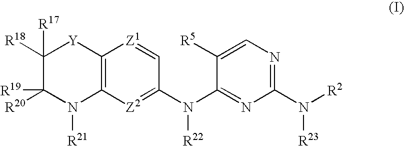

or a salt, solvate, hydrate or N-oxide thereof, wherein R², R⁵, R¹⁷⁻²⁴, Y, Z¹, and Z² are as defined herein.

In a fifth aspect, the invention provides methods for treating a disease or condition caused by a mutation in RET kinase, comprising administering to a subject in need of such treatment an amount of a compound according to formula (II) or (III),

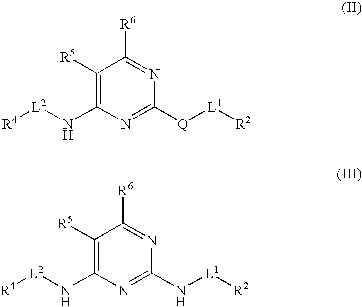

or a salt, solvate, hydrate or N-oxide thereof, wherein L¹, L², Q, R², R⁴, R⁵, and R⁶ are as defined herein.

In another aspect, the invention provides methods of inhibiting proliferation of a thyroid tumor cell, comprising, administering to a tumor cell an amount of a prodrug of a RET kinase inhibitory compound of formula (I), (II), (III), (XI), (XII), or (XIII) effective to inhibit proliferation of the tumor cell.

In another aspect, the invention provides methods of treating a solid thyroid tumor cancer in a subject, comprising administering to a subject an amount of a compound according to structural formula (I), (II), (III), (XI), (XII), or (XIII) effective to treat the solid tumor cancer.

In some embodiments, the cell proliferative disorders treatable with the inhibitor compounds are thyroid cell proliferative disorders. Thyroid cell proliferative disorders treatable with the RET inhibitory compounds include, among others, medullary thyroid carcinomas, papillary thyroid carcinomas, multiple endocrine neoplasia type 2A (MEN2A), parathyroid adenomas, multiple endocrine neoplasia type 2B (MEN2B), familial medullary thyroid carcinoma (FMTC), pheochromocytoma and parathyroid hyperplasia.

In other embodiments, the cell proliferative disorders treatable with the inhibitor compounds are renal tumor cell proliferative disorders. including, but not limited to, renal cell carcinoma, clear cell carcinoma of kidney, and renal cell adenocarcinoma. In some embodiments, the solid tumor cancer is renal cell carcinoma and/or renal cell adenocarcinoma. The tissue of origin for renal cell carcinoma is the proximal renal tubular epithelium. Renal cancer occurs in both a sporadic (nonhereditary) and a hereditary form, and both forms are associated with structural alterations of the short arm of chromosome 3 (3p). Genetic studies of the families at high risk for developing renal cancer led to the cloning of genes whose alteration results in tumor formation. These genes are either tumor suppressors (VHL, TSC) or oncogenes (MET). At least 4 hereditary syndromes associated with renal cell carcinoma are recognized: (1) von Hippel-Lindau (VHL) syndrome, (2) hereditary papillary renal carcinoma (HPRC), (3) familial renal oncocytoma (FRO) associated with Birt-Hogg-Dube syndrome (BHDS), and (4) hereditary renal carcinoma (HRC).

The present disclosure provides 2,4-pyrimidinediamine compounds and prodrugs thereof according to formulae (I), (II), (III), (XI), (XII), and (XIII) that have myriad biological activities, and hence therapeutic uses, compositions comprising the prodrugs, methods and intermediates useful for synthesizing the 2,4-pyrimidinediamine compounds prodrugs and methods of using the 2,4-pyrimidinediamine compounds prodrugs in a variety of in vitro and in vivo contexts, including in the treatment and/or prevention of diseases mediated.

In some aspects, the inhibitor compounds can be used in combination with other cancer treatments. In some embodiments, RET inhibitory compounds are used in combination with other chemotherapeutic agents, including, among others, antimetabolites, alkylating agents, coordination compounds, transcription inhibitors, topoisomerase inhibitors, DNA minor-groove binding compounds, vinca alkyloids, antitumor antibiotics, hormones, and antitumor enzymes.

### BRIEF DESCRIPTION OF THE DRAWINGS

FIG. 1 provides schemes illustrating metabolic pathways of exemplary phosphorous-containing prodrugs.

FIG. 2 provides a scheme illustrating a metabolic pathway of an exemplary cyclic phosphate ester prodrug.

### DETAILED DESCRIPTION OF THE PREFERRED EMBODIMENTS

### Methods of Treatment

The present disclosure provides method of treating cell proliferative disorders by administration of RET kinase inhibitory compounds. The RET gene encodes a single-pass transmembrane receptor tyrosine kinase (RTK). When deregulated, RTKs can become potent oncoproteins. RET is a paradigm of a single RTK gene that induces different types of human cancer depending on the mutation. Germline point mutations in RET cause three related dominantly inherited cancer syndromes: multiple endocrine neoplasia type 2A (MEN2A), MEN2B, familial medullary thyroid carcinoma (FMTC), pheochromocytoma and parathyroid hyperplasia. MEN2 patients are affected by medullary thyroid carcinoma (MTC), a malignant tumor arising from calcitonin-secreting C cells of the thyroid. Pheochromocytoma and parathyroid hyperplasia are present in about 50% and 15-30%, respectively, of MEN2A cases. MEN2B patients can be affected by medullary thyroid carcinoma, pheochromocytomas, musculoskeletal anomalies, sensory deficits and mucosal ganglioneuromas.

RET is a prime target for treatment strategies for thyroid cancer. Strategies such as RNA interference to abrogate gene expression or gene therapy with dominant negative mutants have been envisaged to block RTK function. Moreover, monoclonal antibodies have proven clinical efficacy against RTKs. Several anti-RET antibodies have been reported, but they have not yet been used in treatment. Small molecule TK inhibitors compete with ATP, thereby obstructing autophosphorylation and signal transduction downstream from the targeted kinase.

In the descriptions of the methods herein, the terms used will have their ordinary and common meaning, unless specifically defined otherwise herein.

“Cell proliferative disorder” refers to a disorder characterized by abnormal proliferation of cells. A proliferative disorder does not imply any limitation with respect to the rate of cell growth, but merely indicates loss of normal controls that affect growth and cell division. Thus, in some embodiments, cells of a proliferative disorder can have the same cell division rates as normal cells but do not respond to signals that limit such growth. Within the ambit of “cell proliferative disorder” is neoplasm or tumor, which is an abnormal growth of tissue. Cancer refers to any of various malignant neoplasms characterized by the proliferation of cells that have the capability to invade surrounding tissue and/or metastasize to new colonization sites.

“Inhibition of proliferation” refers to an arrest of cell division, a reduction in the rate of cell division, proliferation and/or growth, and/or induction of cell death. The drugs or prodrugs disclosed herein have been shown to inhibit the proliferation of treated cells as compared to untreated control cells of a similar type. As used herein, inhibition of proliferation can be brought about by any mechanism or combination of mechanisms, and may operate to inhibit proliferation cytostatically or cytotoxically.

“GI₅₀” refers to the concentration of compound at which inhibition of growth of 50% of the population of cells being assayed is observed.

“TGI” refers to the concentration of compound at which total inhibition of growth of cells being assayed is observed.

“LC₅₀” refers to the concentration of compound which results in lethality in 50% of the population of cells being assayed.

“Progroup” refers to a type of protecting group that, when used to mask a functional group within an active 2,4-pyrimidinediamine drug to form a promoiety, converts the drug into a prodrug. Progroups are typically attached to a functional group of the drug via bonds that are cleavable under specified conditions of use (e.g., various physiological conditions). Thus, a progroup is that portion of a promoiety that is cleavable from a molecule under specified conditions (e.g., various physiological conditions) to leave a functional group. As a specific example, an amide promoiety of the formula —NHC(O)CH₃ comprises the progroup —C(O)CH₃ and the functional group —NH₂.

Generally, cell proliferative disorders treatable with the compounds and prodrugs disclosed herein relate to any disorder characterized by aberrant cell proliferation. These include various tumors and cancers, benign or malignant, metastatic or non-metastatic. Specific properties of cancers, such as tissue invasiveness or metastasis, can be targeted using the methods described herein. Cell proliferative disorders include a variety of cancers, including, among others, breast cancer, ovarian cancer, renal cancer, gastrointestinal cancer, kidney cancer, bladder cancer, pancreatic cancer, lung squamous carcinoma, and adenocarcinoma.

It is to be understood that the RET inhibitor compounds can be used independently of any other treatment, or used in combination with other cancer treatment regimens, including surgery, radiology, or other chemotherapies. Accordingly, in some embodiments, the RET kinase inhibitors can be used in combination with other chemotherapeutic agents. Combination treatments with RET inhibitors can target different cellular components by appropriate choice of the second chemotherapeutic agent.

Various chemotherapeutic agents can be used in combination with RET kinase inhibitors to treat cell proliferative disorders. These chemotherapeutic agents can be general cytotoxic agents or target a specific cellular molecule. Various classes of cancer chemotherapeutic agents include, among others, antimetabolites, agents that react with DNA (e.g., alkylating agents, coordination compounds, etc.), inhibitors of transcription enzymes, topoisomerase inhibitors, DNA minor-groove binding compounds, antimitotic agents (e.g., vinca alkyloids), antitumor antibiotics, hormones, and enzymes. Exemplary alkylating agents include, by way of example and not limitation, mechlorothamine, cyclophosphamide, ifosfamide, melphalan, chlorambucil, ethyleneimines, methylmelamines, alkyl sulfonates (e.g., busulfan), and carmustine. Exemplary antimetabolites include, by way of example and not limitation, folic acid analog methotrexate; pyrimidine analogs fluorouracil, cytosine arabinoside; and purine analogs mercaptopurine, thioguanine, and azathioprine. Exemplary vinca alkyloids include, by way of example and not limitation, vinblastine, vincristine, paclitaxel, and colchicine. Exemplary antitumor antibiotics include, by way of example and not limitation, actinomycin D, daunorubicin, and bleomycin. An exemplary enzyme effective as anti-neoplastic agent is L-asparaginase. Exemplary coordination compounds include, by way of example and not limitation, cisplatin and carboplatin. Exemplary hormones and hormone related compounds include, by way of example and not limitation, adrenocorticosteroids prednisone, and dexamethasone; aromatase inhibitors amino glutethimide, formestane, and anastrozole; progestin compounds hydroxyprogesteron caproate, medroxyprogesterone; and anti-estrogen compound tamoxifen. Exemplary topoisomerase inhibitors include, by way of example and not limitation, amsacrine (m-AMSA); mitoxantrone, topotecan, irinotecan, and camptothecin.

These and other useful anti-cancer compounds are described in Merck Index, 13th Ed. (O'Neil, M. J. et al., ed) Merck Publishing Group (2001) and Goodman and Gilmans The Pharmacological Basis of Therapeutics, 10th Edition, Hardman, J. G. and Limbird, L. E. eds., pg. 1381-1287, McGraw Hill, (1996), both of which are incorporated herein by reference.

Other anti-proliferative compounds useful in combination with the RET inhibitor compounds include, by way of example and not limitation, antibodies directed against growth factor receptors (e.g., anti-Her2); cytokines such as interferon-α and interferon-γ, interleukin-2, and GM-CSF; and antibodies for cell surface markers (e.g., anti-CTLA-4. anti-CD20 (rituximab); anti-CD33). When antibodies against cell surface markers are used, a chemotherapeutic agent can be conjugated to it for specific targeting to the tumor cell. Suitable conjugates include radioactive compounds (e.g., radioactive metal bound to an antibody conjugated chelator), cytotoxic compounds, and drug activating enzymes (e.g., allinase, peptidases, esterases, catalytic antibodies, etc.) (see, e.g., Arditti et al., 2005, Mol. Cancer. Therap. 4(2):325-331; U.S. Pat. No. 6,258,360; incorporated herein by reference)

In some embodiments, the RET inhibitors can be used with a second kinase inhibitor that targets an oncogenic kinase different from RET. Given that RET inhibitors are disclosed herein for the treatment of medullary thyroid carcinomas, papillary thyroid carcinomas, multiple endocrine neoplasia type 2A (MEN2A), parathyroid adenomas, multiple endocrine neoplasia type 2B (MEN2B), or familial medullary thyroid carcinoma (FMTC), other compatible kinase inhibitors used for treating thyroid cancers can also be used. Examples include inhibitors of kinases associated with cell proliferative disorders such as, but not limited to the inhibitors of the kinases, Aurora-A, AKT, CDK1/cyclinB, CDK2/cyclinA, CDK3/cyclinE, CDK5/p35, CDK6/cyclinD3, CDK7/cyclinH/MAT1, CHK1, CHK2, EGFR, c-RAF, RAS, cSRC, Yes, Fyn, Lck, Fes, Lyn, Bmx, FGFR3, GSK3α, GSK3β, P13, IGF-1R, MAPK2, MAPKAP-K2, JNK, MEK1, p70S6K, PAK2, PDGFRα, PDGFRβ, PDK1, PKA, PKCε, PKC, PKD2, VEGF, PRAK, PRK2, ROCK-II, Rsk1, Rsk2, Rsk3, and SGK.

As further described herein, the administration of other chemotherapeutic agents can be done in the form of a composition, or administered adjunctively in combination with the RET inhibitor. When provided adjunctively, the chemotherapeutic agents can be administered simultaneously with or sequentially with administration of the RET inhibitor.

### DEFINITIONS

In reference to various inhibitors, the terms used to describe the compounds and prodrugs will have their ordinary and common meaning as used by those in the art unless a different definition is provided herein or is provided in the references describing the specific inhibitor compounds.

Those of skill in the art will appreciate that some of the drug and prodrug compounds of the methods described herein may exhibit the phenomena of tautomerism, conformational isomerism, geometric isomerism and/or optical isomerism. For example, the drug and prodrug compounds may include one or more chiral centers and/or double bonds and as a consequence may exist as stereoisomers, such as double-bond isomers (i.e., geometric isomers), enantiomers and diasteromers and mixtures thereof, such as racemic mixtures. As another example, the drug and prodrug compounds may exist in several tautomeric forms, including the enol form, the keto form and mixtures thereof. As the various compound names, formulae and compound drawings within the specification and claims can represent only one of the possible tautomeric, conformational isomeric, optical isomeric or geometric isomeric forms, it should be understood that the invention encompasses any tautomeric, conformational isomeric, optical isomeric and/or geometric isomeric forms of the drug and prodrug compounds having one or more of the utilities described herein, as well as mixtures of these various different isomeric forms. In cases of limited rotation around the 2,4-pyrimidinediamine core structure, atropisomers are also possible and are also specifically included in the drug and prodrug compounds of the methods herein described.

“Alkyl” by itself or as part of another substituent refers to a saturated or unsaturated, branched, straight-chain or cyclic monovalent hydrocarbon group having the stated number of carbon atoms (i.e., C₁-C₆ means from one to six carbon atoms) derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. The expression “lower alkyl” refers to alkyl groups composed of from 1 to 6 carbon atoms.

“Alkanyl” by itself or as part of another substituent refers to a saturated branched, straight-chain or cyclic alkyl group. Typical alkanyl groups include, but are not limited to, methanyl; ethanyl; propanyls such as propan-1-yl, propan-2-yl (isopropyl), cyclopropan-1-yl, etc.; butyanyls such as butan-1-yl, butan-2-yl (sec-butyl), 2-methyl-propan-1-yl (isobutyl), 2-methyl-propan-2-yl (t-butyl), cyclobutan-1-yl, etc.

“Alkenyl” by itself or as part of another substituent refers to an unsaturated branched, straight-chain or cyclic alkyl group having at least one carbon-carbon double bond derived by the removal of one hydrogen atom from a single carbon atom of a parent alkene. The group can be in either the cis or trans (i.e., Z- or E-) conformation about the double bond(s). Typical alkenyl groups include, but are not limited to, ethenyl (i.e., vinyl); propenyls such as prop-1-en-1-yl, prop-1-en-2-yl, prop-2-en-1-yl (i.e., allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.

“Alkynyl” by itself or as part of another substituent refers to an unsaturated branched, straight-chain or cyclic alkyl group having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.

“Alkoxy” by itself or as part of another substituent refers to an alkyl group, as defined herein, attached to its parent moiety via an oxygen atom. “Lower alkoxy” by itself or as part of another substituent refers to a lower alkyl group, as defined herein, attached to its parent moiety via an oxygen atom.

“Parent Aromatic Ring System” refers to an unsaturated cyclic or polycyclic ring system having a conjugated π-electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Further examples of parent aromatic ring systems include, but are not limited to, anthracene, azulene, benzene, fluorene, indane, indene, naphthalene, phenanthrene, pyrene, cyclopentadienyl, and the like.

“Aryl” by itself or as part of another substituent refers to a monovalent aromatic hydrocarbon group having the stated number of carbon ring atoms (i.e., C₅-C₁₄ means from 5 to 14 carbon ring atoms) derived by the removal of one hydrogen atom from a single carbon atom of a parent aromatic ring system. Typical aryl groups include, but are not limited to, groups derived from anthracene, azulene, benzene, fluorene, indane, indene, naphthalene, phenanthrene, pyrene, cyclopentadienyl, and the like. In preferred embodiments, the aryl group is (C₅-C₁₄) aryl, with (C₅-C₁₀) being even more preferred. Particularly preferred aryls are cyclopentadienyl, phenyl and naphthyl.

“Arylalkyl” by itself or as part of another substituent refers to an acyclic alkyl group in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In preferred embodiments, the arylalkyl group is (C₆-C₁₆) arylalkyl, e.g., the alkanyl, alkenyl or alkynyl moiety of the arylalkyl group is (C₁-C₆) and the aryl moiety is (C₅-C₁₀). In particularly preferred embodiments the arylalkyl group is (C₆-C₁₃), e.g., the alkanyl, alkenyl or alkynyl moiety of the arylalkyl group is (C₁-C₃) and the aryl moiety is (C₅-C₁₀).

“Aryloxy” by itself or as part of another substituent refers to an aryl group, as defined herein, attached to its parent moiety via an oxygen atom.

“Bridged cycloalkyl” refers to cycloalkyl groups, as defined herein, containing a linking moiety connecting any two non-adjacent carbon atoms within the cycloalkyl ring. Typical linkers include, but are not limited to, methano, ethano, propano, —O—, —NH—, and the like. Typical bridged cycloalkyl groups include, but are not limited to, norbornene, norbornane, norbornadiene, bicyclo[3.3.1]nonane, 9-oxabicyclo[3.3.1]nonane, 3-oxabicyclo[3.3.1]nonane, 8-azabicyclo[3.2.1]octane, 3-azabicyclo[3.2.1]octane, 7-oxabicyclo[2.2.1]heptane, bicyclo[2.2.2]octane, bicyclo[2.2.2]oct-2-ene, 7-oxabicyclo[2.2.1]hept-2-ene, and 7-oxabicyclo[2.2.1]hepta-2,5-diene.

“Cycloalkyl” and “Heterocycloalkyl” by themselves or as part of another substituent refer to cyclic versions of “alkyl” and “heteroalkyl” groups, respectively. For heteroalkyl groups, a heteroatom can occupy the position that is attached to the remainder of the molecule. Typical cycloalkyl groups include, but are not limited to, cyclopropyl; cyclobutyls such as cyclobutanyl and cyclobutenyl; cyclopentyls such as cyclopentanyl and cyclopentenyl; cyclohexyls such as cyclohexanyl and cyclohexenyl; and the like. Typical heterocycloalkyl groups include, but are not limited to, tetrahydrofuranyl (e.g., tetrahydrofuran-2-yl, tetrahydrofuran-3-yl, etc.), piperidinyl (e.g., piperidin-1-yl, piperidin-2-yl, etc.), morpholinyl (e.g., morpholin-3-yl, morpholin-4-yl, etc.), piperazinyl (e.g., piperazin-1-yl, piperazin-2-yl, etc.), and the like. The expression “lower cycloalkyl” refers to a cycloalkyl group composed of from 3 to 8 carbon atoms.

“Cycloalkylalkyl” and “Heterocycloalkylalkyl” as used herein means a cycloalkyl or heterocycloalkyl group as defined herein, respectively, attached to the parent moiety via an alkyl group, as defined herein.

“Halo” or “halogen” as used herein means —F, —Cl, —Br, or —I.

“Parent Heteroaromatic Ring System” refers to a parent aromatic ring system in which one or more carbon atoms are each independently replaced with the same or different heteroatoms or heteroatomic groups. Typical heteroatoms or heteroatomic groups to replace the carbon atoms include, but are not limited to, N, NH, P, O, S, Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Also included in the definition of “parent heteroaromatic ring system” are those recognized rings that include substituents, such as benzopyrone. Typical parent heteroaromatic ring systems include, but are not limited to, arsindole, benzodioxan, benzofuran, benzopyrone, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Heteroaryl” by itself or as part of another substituent refers to a monovalent heteroaromatic group having the stated number of ring atoms (i.e., “5-14 membered” means from 5 to 14 ring atoms) derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some embodiments, the heteroaryl group is a 5-14 membered heteroaryl or a 5-10 membered heteroaryl.

“Heteroarylalkyl” by itself or as part of another substituent refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. In some embodiments, the heteroarylalkyl group is a 6-20 membered heteroarylalkyl, e.g., the alkanyl, alkenyl or alkynyl moiety of the heteroarylalkyl is 1-6 membered and the heteroaryl moiety is a 5-14-membered heteroaryl. In particularly preferred embodiments, the heteroarylalkyl is a 6-13 membered heteroarylalkyl, e.g., the alkanyl, alkenyl or alkynyl moiety is 1-3 membered and the heteroaryl moiety is a 5-10 membered heteroaryl.

“Substituted Alkyl, Aryl, Arylalkyl, Cycloalkyl, Cycloalkyalkyl, Heteroaryl Heteroarylalkyl, Heterocycloalkyl, or Heterocycloalkylalkyl” refers to an alkyl, aryl, arylalkyl, cycloalkyl, cycloalkylalkyl, heteroaryl, heteroarylalkyl, heterocycloalkyl, or heterocycloalkylalkyl group in which one or more hydrogen atoms is replaced with another substituent group. Exemplary substituent groups include, but are not limited to, —OR′, —SR′, —NR′R′, —NO₂, —NO, —CN, —CF₃, halogen (e.g., —F, —Cl, —Br and —I), —C(O)R′, —C(O)OR′, —C(O)NR′, —S(O)₂R′, —S(O)₂NR′R′, where each R′ is independently hydrogen or (C₁-C₆) alkyl.

“Prodrug” refers to a derivative of an active compound (drug) that, under the conditions of use, such as within the body, is transformed to the active drug. Prodrugs are frequently, but not necessarily, pharmacologically inactive until converted into the active drug. Prodrugs are typically obtained by masking a functional group in the drug believed to be in part required for activity with a progroup (defined below) to form a promoiety which undergoes a transformation, such as chemical cleavage, under the specified conditions of use to transform the functional group, and hence release the active drug. The cleavage of the promoiety can proceed spontaneously, such as by way of a hydrolysis reaction, or it can be catalyzed or induced by another agent, such as by an enzyme, by light, by acid, or by a change of or exposure to a physical or environmental parameter, such as a change of temperature. The agent can be endogenous to the conditions of use, such as an enzyme present in the cells to which the prodrug is administered or the acidic conditions of the stomach, or it can be supplied exogenously.

A wide variety of progroups, as well as the resultant promoieties, suitable for masking functional groups in the active drugs to yield prodrugs are well-known in the art. For example, a hydroxyl functional group can be protected as a sulfonate, ester or carbonate promoiety, each of which can be hydrolyzed in vivo to provide the hydroxyl group. An amino functional group can be protected as an amide, carbamate, imine, urea, phosphenyl, phosphoryl or sulfenyl promoiety, each of which can be hydrolyzed in vivo to provide the amino group. A carboxyl group can be protected as an ester (including silyl esters and thioesters), amide or hydrazide promoiety, each of which can be hydrolyzed in vivo to provide the carboxyl group. Other specific examples of suitable progroups and their respective promoieties will be apparent to those of skill in the art.

In some embodiments, the inhibitor compound can be selective for RET kinase. A “RET kinase selective inhibitory compound” refers to a compound displaying selectivity for RET, which is defined as the ratio of an IC₅₀ for a reference kinase over an IC₅₀ for RET kinase in a defined set of assays. Generally the RET kinase selective inhibitory compound can have a selectivity for RET kinase that is greater than about 10 times, greater than about 50 times, greater than about 100 times, greater than about 1000 times, or higher more selective. The reference kinase can be any kinase, particularly a kinase with activity associated with cell proliferative disorders or with an undesired clinical effect when inhibited. Reference kinases may include, by way of example and not limitation, kinases associated with cell proliferative disorders such as Aurora-A, AKT, CDK1/cyclinB, CDK2/cyclinA, CDK3/cyclinE, CDK5/p35, CDK6/cyclinD3, CDK7/cyclinH/MAT1, CHK1, CHK2, EGFR, c-RAF, RAS, cSRC, Yes, Fyn, Lck, Fes, Lyn, Bmx, FGFR3, GSK3α, GSK3β, P13, IGF-1R, MAPK2, MAPKAP-K2, JNK, MEK1, p70S6K, PAK2, PDGFRβ, PDGFRβ, PDK1, PKA, PKCε, PKC, PKD2, VEGF, PRAK, PRK2, ROCK-II, Rsk1, Rsk2, Rsk3, SGK. Various assays for each of the kinases will be apparent to the skilled artisan. For example, Aurora kinase activities can use natural or synthetic substrates (e.g., fluorescent peptides, Histone H3) in in vitro assays, or measurement of phosphorylated products in cells (Walter et al., 2000, Oncogene 19(42):4906-16). Kinase activities can be detected using various approaches, including, by way of example and not limitation, immunoprecipitation (e.g., Cyclex Aurora A kinase Assay; MBL Corp, Woburn, Mass., USA) mobility shift (e.g., Caliper Technologies, Mountain View, Calif., USA), autofluorescent fusion protein substrates (e.g., U.S. Pat. No. 6,248,550), and FRET based assays (Z-LYTE®; Invitrogen, Calif., USA). As will be appreciated by the skilled artisan, other active kinases involved in aberrant cell proliferation or undesired clinical effects, when inhibited, can be used to determine the selectivity of a kinase inhibitor for RET.

Various kinase inhibitors can be used in the methods herein, and is meant to include, where applicable, the salts, hydrates, solvates, and N-oxides of the corresponding inhibitor compounds.

RET Kinase Inhibitor Compounds

According the first aspect, drug compounds useful in the methods of the invention are compounds according to structural formula (XI):

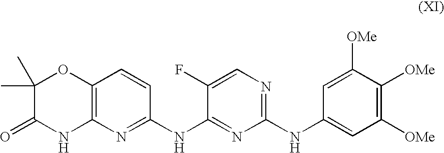

including pharmaceutically acceptable salts, hydrates, solvates and N-oxides thereof.

In one embodiment of the first aspect, the invention provides pharmaceutically acceptable salts of compounds according to structural formula (XI). Such salts may be, for example, acid addition salts of at least one of the following acids: benzenesulfonic acid, citric acid, α-glucoheptonic acid, D-gluconic acid, glycolic acid, lactic acid, malic acid, malonic acid, mandelic acid, phosphoric acid, propanoic acid, succinic acid, sulfuric acid, tartaric acid (d, l, or dl), tosic acid (toluenesulfonic acid), valeric acid, palmitic acid, pamoic acid, sebacic acid, stearic acid, lauric acid, acetic acid, adipic acid, carbonic acid, 4-chlorobenzenesulfonic acid, ethanedisulfonic acid, ethylsuccinic acid, fumaric acid, galactaric acid (mucic acid), D-glucuronic acid, 2-oxo-glutaric acid, glycerophosphoric acid, hippuric acid, isethionic acid (ethanolsulfonic acid), lactobionic acid, maleic acid, 1,5-naphthalene-disulfonic acid, 2-naphthalene-sulfonic acid, pivalic acid, terephthalic acid, thiocyanic acid, cholic acid, n-dodecyl sulfate, 3-hydroxy-2-naphthoic acid, 1-hydroxy-2-naphthoic acid, oleic acid, undecylenic acid, ascorbic acid, (+)-camphoric acid, d-camphorsulfonic acid, dichloroacetic acid, ethanesulfonic acid, formic acid, hydriodic acid, hydrobromic acid, hydrochloric acid, methanesulfonic acid, nicotinic acid, nitric acid, orotic acid, oxalic acid, picric acid, L-pyroglutamic acid, saccharine, salicylic acid, gentisic acid, and/or 4-acetamidobenzoic acid.

In one embodiment of the first aspect, the invention provides pharmaceutically acceptable salts of compounds according to structural formula (XI), wherein the acid addition salt is a hydrogen chloride addition salt or a sulfonic acid addition salt. Examples of sulfonic acid addition salts include, but are not limited to, benzenesulfonate (besylate), trifluoromethylsulfonate (triflate), 4-bromobenzenesulfonate (brosylate), p-toluenesulfonate (tosylate), methylsulfonate (mesylate), 4-hydroxybenzenesulfonate, 2,4,6-trimethylbenzenesulfonate, pyridine-3-sulfonate, p-ethylbenzenesulfonate, 1,2-ethanedisulfonate, (1R)-10-camphorsulfonate, and (1S)-10-camphorsulfonate.

In another embodiment, the invention provides pharmaceutically acceptable solvates of compounds according to structural formula (XI). Such solvates, may include a stoichiometric (or above) or substoichiometric ratio of compound of the invention to solvate. Solvates may include water (hydrates), methanol, ethanol, dimethylsulfoxide, isopropanol, ethyl acetate, ethanolamine, acetonitrile, and mixtures thereof.

RET Kinase Inhibitor Prodrugs

According to the second aspect, the present invention provides prodrugs compounds of formula (XII),

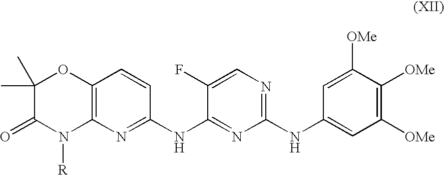

including pharmaceutically acceptable salts, hydrates, solvates and/or N-oxides thereof, wherein R represents a progroup, as described below.

Prodrugs are derivatives of drug compounds that require a transformation under the conditions of use, such as within the body, to release the active drug. Prodrugs are frequently, but not necessarily, pharmacologically inactive until converted into the active drug. Prodrugs are typically obtained by masking a functional group in the drug believed to be in part required for activity with a progroup (defined below) to form a promoiety which undergoes a transformation, such as cleavage, under the specified conditions of use to release the functional group, and hence the active drug.

The cleavage of the promoiety may proceed spontaneously, such as by way of a hydrolysis reaction, or it may be catalyzed or induced by another agent, such as by an enzyme, by light, by acid, or by a change of or exposure to a physical or environmental parameter, such as a change of temperature. The agent may be endogenous to the conditions of use, such as an enzyme present in the cells to which the prodrug is administered or the acidic conditions of the stomach, or it may be supplied exogenously.

Such prodrugs may be active in their prodrug form, or may be inactive until converted under physiological or other conditions of use to an active drug form. For example, primary or secondary amino groups may be included in an amide promoiety that cleaves under conditions of use to generate the primary or secondary amino group.

Thus, the prodrugs include special types of protecting groups, termed “progroups,” that cleave under the conditions of use to yield an active 2,4-pyrimidinediamine drug compound. Myriad progroups suitable for masking such functional groups to yield promoieties that are cleavable under the desired conditions of use are known in the art (see, for example, Greene and Wuts, Greene's Protective Groups in Organic Synthesis, 4th Ed., Wiley-Interscience:New York, 2006; and Testa and Mayer, Hydrolysis in Drug and Prodrug Metabolism: Chemistry, biochemistry, and Enzymology, Academic Press: New York, 2003). All of these progroups, alone or in combinations, may be included in the prodrugs. Specific examples of promoieties that yield primary or secondary amine groups that can be included in the prodrugs include, but are not limited to amides, carbamates, imines, ureas, phosphenyls, phosphoryls and sulfenyls.

The class of 2,4-pyrimidinediamine drug and prodrug compounds of formulae (XI) and (XII) have been previously described in detail in U.S. application Ser. No. 11/337,049 filed Jan. 19, 2006 (US2006/0211657), and U.S. application Ser. No. 10/913,270 filed Aug. 6, 2004 (US2005/0113398), the disclosures of which are incorporated herein by reference in their entirety.

The progroup can include, but is not limited to, a group or moiety that can be converted (e.g., metabolized), under the conditions of use, to yield an unstable α-hydroxymethyl, α-aminomethyl or α-thiomethyl intermediate, which then further converted in vivo to yield the active 2,4-pyrimidinediamine drug. In some embodiments, the progroup can be, for example, an acid labile hydroxyalkyl-containing progroup, an acid labile phosphate containing progroup, or a salt thereof. In some embodiments, the progroup is —CH₂—O—P(O)(OH)₂, including ionized forms (e.g., —CH₂—O—P(O)(OH)O⁻ and —CH₂—O—P(O)(O⁻)₂) and salts thereof. Preferred prodrug groups include any of those described with respect to the variable R^{p}, below. In one example, the prodrug comprises a base addition salt of the aforementioned phosphonate progroups. In some embodiments this salt form exists as a hydrate. In one exemplary embodiment, the compound of the invention is a disodium salt hydrate, more specifically N4-(2,2-dimethyl-4-[(dihydrogen phosphonoxy)methyl]-3-oxo-5-pyrido[1,4]oxazin-6-yl)-5-fluoro-N2-(3,4,5-trimethoxyphenyl)-2,4-pyrimidinediamine disodium salt hexahydrate.

According to the third aspect, drug compounds useful in the methods of the invention are compounds according to structural formula (XIII),

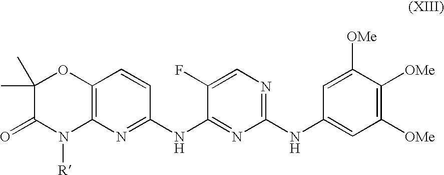

including pharmaceutically acceptable salts, hydrates, solvates and/or N-oxides thereof, wherein R′ is independently hydrogen or a progroup.

Specific embodiments of progroups, R′, include those discussed below with respect to the variable R^{p}.

In one embodiment of the fourth aspect of the invention, the prodrugs for use in the methods of the invention are compounds according to structural formula (I)

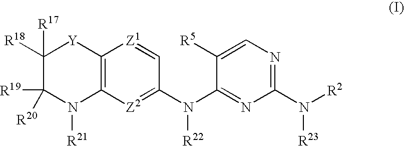

including salts, solvates, hydrates and N-oxides thereof, wherein:
Y is CH₂, NR²⁴, O, S, S(O), or S(O)₂;
Z¹ and Z² are independently CH or N;
R² is lower alkyl optionally substituted with one or more of the same or different R⁸ groups, lower cycloalkyl optionally substituted with one or more of the same or different R⁸ groups, cyclohexyl optionally substituted with one or more of the same or different R⁸ groups, 3-8 membered heterocycloalkyl optionally substituted with one or more of the same or different R⁸ groups, (C₆-C₁₄) aryl optionally substituted with one or more of the same or different R⁸ groups, phenyl optionally substituted with one or more of the same or different R⁸ groups, or 5-15 membered heteroaryl optionally substituted with one or more of the same or different R⁸ groups;
R⁵ is halo (e.g. fluoro), cyano, nitro, trihalomethyl, or trifluoromethyl;
each R⁸ is independently R^{a}, R^{b}, —B(OR^{a})₂, —B(R^{c}R^{c})₂, —(CH₂)ₘ—R¹, —(CHR^{a})ₘ—R^{b}, —O—(CH₂)ₘ—R^{b}, S—(CH₂)ₘ—R^{b}, —O—CHR^{a}R^{b}, —O—CR^{a}(b)₂, —O—(CHR^{a})ₘ—R^{b}, —O— (CH₂)ₘ—CH[(CH₂)ₘR^{b}]R^{b}, —S—(CHR^{a})ₘ—R^{b}, —O—C(O)NH—(CH₂)ₘ—R^{b}, —C(O)NH—(CHR^{a})ₘ—R^{b}, —O—(CH₂)ₘ—C(O)NH—(CH₂)ₘ—R^{b}, —S—(CH₂)ₘ—C(O)NH—(CH₂)ₘ—R^{b}, —O—(CHR^{a})ₘ—C(O)NH—(CHR^{a})_R^{b}, S—(CHR^{a})ₘ—C(O)NH—(CHR^{a})ₘ—R^{b}, —NH—(CH₂)ₘ—R^{b}, —NH—(CHR^{a})ₘ—R^{b}, —NH—[(CH₂)ₘR^{b}], —NH—[(CH₂)ₘR^{b}]₂, NH—C(O)—NH—(CH₂)ₘ—R^{b}, —NH—C(O)—(CH₂)ₘ—CHR^{b}R^{b}, NH—(CH₂)ₘ—C(O)—NH—(CH₂)ₘ—R^{b}, R^{a} substituted with one to four of the same or different R^{a} or R^{b}; or —OR^{a} substituted with one or more of the same or different R^{a} or R^{b},
R¹⁷ and R¹⁸ are independently hydrogen, halogen, fluoro, lower alkyl, or methyl; or
R¹⁷ and R¹⁸ taken together form an oxo (═O) group or, together with the carbon atom to which they are attached, a spirocycle containing from 3 to 7 carbon atoms;
R¹⁹ and R²⁰ are independently hydrogen, lower alkyl, or methyl; or
R¹⁹ and R²⁰ taken together form an oxo (═O) group or, together with the carbon atom to which they are attached, a spirocycle containing from 3 to 7 carbon atoms;
each R^{a} is independently hydrogen, lower alkyl, lower cycloalkyl, cyclohexyl, (C₄-C₁₁) cycloalkylalkyl, (C₆-C₁₀) aryl, phenyl, (C₇-C₁₆) arylalkyl, benzyl, 2-6 membered heteroalkyl, 3-8 membered heterocycloalkyl, morpholinyl, piperazinyl, homopiperazinyl, piperidinyl, 4-11 membered heterocycloalkylalkyl, 5-10 membered heteroaryl, or 6-16 membered heteroarylalkyl;
each R^{b} is independently ═O, —OR^{a}, (C₁-C₃) haloalkyloxy, ═S, —SR^{a}, ═NR^{a}, ═NOR^{a}, —NR^{c}R^{c}, halogen, —CF₃, —CN, —NC, —OCN, —SCN, —NO, —NO₂, ═N₂, —N₃, —S(O)R^{a}, —S(O)₂R^{a}, —S(O)₂OR^{a}, —S(O)NR^{c}R^{c}, —S(O)₂NR^{c}R^{c}, —OS(O)₂R^{a}, OS(O)₂OR^{a}, —OS(O)₂NR^{c}R^{c}, —C(O)R^{a}, —C(O)OR^{a}, —C(O)NR^{c}R^{c}, —C(NH)NR^{c}R^{c} —C(NR^{a})NR^{c}R^{c}, —C(NOH)R^{a}, —C(NOH)NR^{c}R^{c}, —OC(O)R^{a}, —OC(O)OR^{a}, —OC(O)NR^{c}R^{c}, —OC(NH)NR^{c}R^{c}, —OC(NR^{a})NR^{c}R^{c}, —[NHC(O)]ₙR^{a}, —[NR^{a}C(O)]ₙR^{a}, —[NHC(O)]ₙOR^{a}, —[NR^{a}C(O)]ₙOR^{a}, —[NHC(O)]ₙNR^{c}R^{c}, —[NR^{a}C(O)]ₙNR^{c}R^{c}, —[NHC(NH)]ₙNR^{c}R^{c}, or —[NR^{a}C(NR^{a})]ₙNR^{c}R^{c};
each R^{c} is independently R^{a},
or two R^{c} bonded to the same nitrogen atom, taken together with the nitrogen atom to which they are both attached, form a 5 to 8-membered heterocycloalkyl or heteroaryl group comprising one or more of the same or different additional heteroatoms and optionally substituted with one to four of the same or different R^{a} groups;
R²¹, R²² and R²³ are each independently hydrogen or progroup R^{p};
R²⁴ is hydrogen, lower alkyl, or progroup R^{p};
each m is independently 1, 2, or 3; and
each n is independently 0, 1, 2, or 3,
provided that at least one of R²¹, R²², R²³ and R²⁴ is R^{p}.

In the prodrugs described herein, and in particular in the prodrugs of structural formula (I), R²¹, R²² and R²³ each represent either hydrogen or a progroup R^{p}. Also, R²⁴ represents hydrogen, a lower alkyl or a progroup R^{p}. Thus, the prodrugs can include a single R^{p} progroup, two R^{p} progroups, three R^{p} progroups, or even more R^{p} progroups, depending, in part, on the identity of Y and whether the R² substituent includes any R^{p} progroups. In some embodiments, it is preferred that the prodrugs described herein, and in particular the prodrugs of structural formula (I), include only one R^{p} group. Without intending to be bound by any theory of operation, it is possible that the different R^{p} groups in prodrugs including more than one R^{p} progroup may metabolize at different rates. Prodrugs including a single R^{p} progroup would avoid such differential metabolic kinetics. A specific embodiment of prodrugs according to structural formula (I) that include a single progroup R^{p} are compounds according to structural formula (Ia):

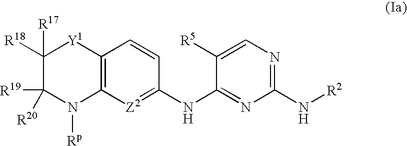

wherein Y¹ is CH₂, NR²⁴, O, S, S(O), or S(O)₂; and Z², R², R⁵, R¹⁷, R¹⁸, R¹⁹, R²⁰, R²⁴ and R^{p} are as previously defined, with the proviso that R² does not include any R^{p} groups.

A specific embodiment of prodrugs according to structural formula (Ia) that include a single progroup R^{p} are compounds according to structural formula (Ib):

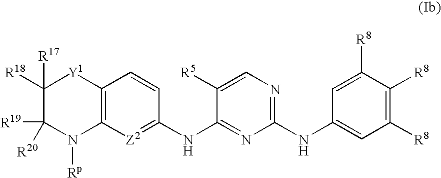

wherein Y¹ is CH₂, NR²⁴, O, S, S(O), or S(O)₂; and Z², R⁵, R⁸, R¹⁷, R¹⁸, R¹⁹, R²⁰, R²⁴ and R^{p} are as previously defined, with the proviso that each R⁸ is not an R^{p} group.

A specific embodiment of prodrugs according to structural formula (Ib) that include a single progroup R^{p} are compounds according to structural formula (Ic):

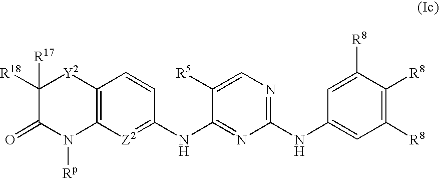

wherein Y² is O, S, S(O), or S(O)₂; and Z², R⁵, R⁸, R¹⁷, R¹⁸, R²⁴ and R^{p} are as previously defined, with the proviso that each R⁸ is not an R^{p} group.

The identity of any R^{p} progroups present in the prodrugs described herein is not critical for success, provided that it hydrolyzes under the conditions of use to yield the active 2,4-pyrimidinediamine compound.

It has recently been discovered that a phosphate-containing prodrug according to the structure illustrated below (Compound P-1):

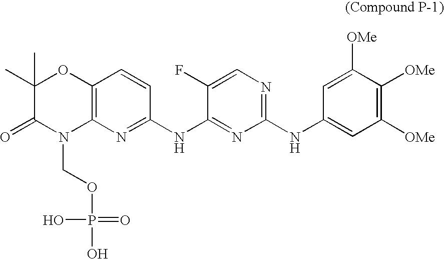

metabolizes in vivo to the corresponding active 2,4-pyrimidinediamine compound (Compound 1), illustrated below:

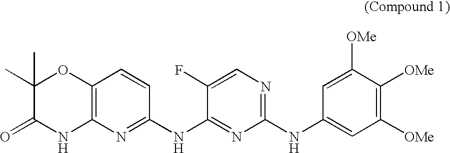

While not intending to be bound by any particular theory operation, it is believed that prodrug compound P-1 metabolizes to active Compound 1 via the corresponding hydroxymethylamine intermediate (1-1) illustrated below:

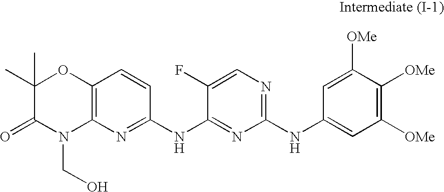

Such hydroxymethylamines, a hemi-aminal group, are known to be unstable under physiological conditions and various pH ranges where they hydrolyze in vivo to yield formaldehyde and the active drug substance. Thus, prodrugs like P-1 are believed to be metabolized in vivo, for example by the acidic conditions of the stomach and/or by enzymes present in the digestive tract or other organs and/or tissues or fluids with the body, to yield the hydroxymethylamine intermediate illustrated above which will likewise metabolize to the active 2,4 pyrimidinediamine drug, such as those of formula (XI) (supra). Prodrug P-1 and salts and hydrates thereof are described in International Patent Application Publication WO/2008/064274, which is herein incorporated in its entirety.

Moreover, it is expected that the amino and thio analogs of this hydroxymethylamine intermediate, will be similarly unstable at physiological conditions and also hydrolyze in vivo to the active 2,4-pyrimdiendiamine drug. Accordingly, the corresponding amino and thio compounds, as well as compounds in which the α-amino and α-thio groups are masked with “protecting” groups that are removed under physiological conditions of use to yield the α-amino and α-thio groups, can serve as suitable prodrugs.

Thus, in some embodiments, the progroup(s) R^{p} in the prodrugs of structural formulae (I), (Ia), (Ib), and (Ic) are each independently R^{p 1} or R^{p2}, wherein
R^{p1} is —C(═X²)—X¹—(CR⁵⁵R⁶⁵)_{q}—R⁷⁵, wherein
X¹ is O, S, or NR¹¹, wherein each R¹¹ is independently H or lower alkyl;
X² is O or S;
R⁵⁵ and R⁶⁵ are each independently H, OH, —OR¹¹, NR¹⁵R¹⁵, halo, lower alkyl, —C(O)O-alkyl, —C(O)OH, —OP(═O)(OR¹¹)₂, —OC(═O)OR¹¹, —OC(═O)R¹¹, cycloalkyl, aryl, heteroaryl or together form an oxo, wherein
each R¹⁵ is independently selected from H, lower alkyl, prenyl, allyl, —C(O)O— alkyl, cycloalkyl, aryl, heteroaryl, alkaryl and alkheteroaryl,
or two of R¹⁵ combine to form an optionally substituted heterocycloalkyl wherein each optionally substituted group is independently selected from R^{b};
R⁷⁵ is straight or branched, saturated or unsaturated alkyl, allyl, cycloalkyl, cycloalkyl, heterocycloalkyl, aryl, heteroaryl, prenylalkaryl, or heteroarylalkyl, each of which is optionally substituted wherein each optionally substituted with one or more (e.g., with 1, 2, or 3 groups) R¹ groups;
q is an integer from 0 to 10; and
R^{p2} is —C(R^{d}R^{d})_{y}-A-R³, wherein
each R^{d} is independently hydrogen, cyano, —C(O)R^{e1}, —C(O)OR^{e1}, —C(O)NR^{e1}R^{e1}, —C(OR^{e1})(OR^{e1}), optionally substituted (C₁-C₂₀) alkyl, (C₁-C₂₀) perfluoroalkyl, optionally substituted (C₇-C₃₀) arylalkyl, or optionally substituted 6-30 membered heteroarylalkyl, wherein
each R^{e1} is independently hydrogen, alkyl, aryl, arylalkyl, heteroaryl, or heteroarylalkyl;
y is 1, 2, or 3;
   A is O, S or NR⁵⁰, wherein R⁵⁰ is R^{d} or cycloalkyl; and

R³ is —R^{f}, —C(O)R^{f}, —C(O)O—R^{f}, —C(O)NR^{f}R^{f}, —Si(R^{f})₃, —P(O)(OH)₂, —P(O)(OH)(OR^{e}), —P(O)(OR^{e})₂, —P(OH)₂, —P(OH)(OR^{e}), or —P(OR^{e})₂, wherein
each R^{e} is independently (i) substituted or unsubstituted lower alkyl, substituted or unsubstituted (C₆-C₁₄) aryl, or substituted or unsubstituted (C₇-C₂₀) arylalkyl wherein each is optionally substituted with one or more groups (e.g., 1, 2, or 3 groups) independently selected from R^{b}, or (ii) —(CR^{d}R^{d})_{y}—OR^{f}, —(CR^{d}R^{d})_{y}—O—C(O)R^{f}, —(CR^{d}R^{d})_{y}—O—C(O)OR^{f}, —(CR^{d}R^{d})_{y}—S—C(O)R^{f}, —(CR^{d}R^{d})_{y}—S—C(O)OR^{f}, —(CR^{d}R^{d})_{y}—NH—C(O)R^{f}, —(CR^{d}R^{d})_{y}—NH—C(O)OR^{f}, or —Si(R^{f})₃;
or two R^{e} taken together with the oxygen atoms to which they are attached, form a 5-8 membered heterocycloalkyl group optionally substituted with substituted or unsubstituted lower alkyl, substituted or unsubstituted lower heterocycloalkyl, substituted or unsubstituted (C₆-C₁₄) aryl, substituted or unsubstituted (C₇-C₂₀) arylalkyl, or substituted or unsubstituted 5-14 membered heteroaryl wherein each is optionally substituted with one or more groups (e.g., 1, 2, or 3 groups) independently selected from R^{b};
each R^{f} group is independently hydrogen, optionally substituted lower alkyl, optionally substituted lower heteroalkyl, optionally substituted lower cycloalkyl, optionally substituted lower heterocycloalkyl, optionally substituted (C₆-C₁₀) aryl, optionally substituted 5-10 membered heteroaryl, optionally substituted (C₇-C₁₈) arylalkyl, or optionally substituted 6-18 membered heteroarylalkyl, wherein each is optionally substituted with one or more groups (e.g., 1, 2, or 3 groups) independently selected from R^{b};
or R⁵⁰ and R³ taken together with nitrogen atom to which they are both attached, form a three- to seven-membered ring,
where R^{b} is as defined for formula (I).

In certain embodiments, the progroup(s) R^{p} in the prodrugs of structural formulae (I), (Ia), (Ib), and (Ic) are of the formula —CR^{d}R^{d}-A-R³, wherein
each R^{d} is independently hydrogen, cyano, —C(O)R^{e1}, —C(O)OR^{e1}, —C(O)NR^{e1}R^{e1}, —C(OR^{e1})(OR^{e1}), optionally substituted (C₁-C₂₀) alkyl, (C₁-C₂₀) perfluoroalkyl, optionally substituted (C₇-C₃₀) arylalkyl, or optionally substituted 6-30 membered heteroarylalkyl;
each R^{e1} is independently hydrogen, alkyl (for example lower alkyl), aryl (for example phenyl or naphthyl, arylalkyl (for example benzyl), heteroaryl, or heteroarylalkyl;
A is O, S or NR⁵⁰, wherein
R⁵⁰ is R^{d}, cycloalkyl; and
R³ is a group that, together with A, metabolizes under the conditions of use to yield an intermediate group of the formula —CR^{d}R^{d}AH,
or, R⁵⁰ and R³ taken together with nitrogen atom to which they are both attached, form a three- to seven-membered ring;
wherein R^{d} and A are as previously defined.

As mentioned above, compounds of structural formula (I), (Ia), (Ib) and (Ic) in which the R^{p} groups are of the formula —CR^{d}R^{d}-AH spontaneously hydrolyze in vivo to yield the active 2,4-pyrimidinediamine drug.

The mechanism by which the R³ group metabolizes to yield intermediate group —CR^{d}R^{d}-AH is not critical, and can be caused by, for example, hydrolysis under the acidic conditions of the stomach, and/or by enzymes present in the digestive tract and/or tissues or organs of the body. Indeed, the R³ group(s) can be selected to metabolize at a particular site within the body. For example, many esters are cleaved under the acidic conditions found in the stomach. Prodrugs designed to cleave chemically in the stomach to the active 2,4-pyrimidinediamine can employ progroups including such esters. Alternatively, the progroups can be designed to metabolize in the presence of enzymes such as esterases, amidases, lipolases, phosphatases including ATPases and kinase etc., to yield the intermediate group of formula —CR^{d}R^{d}-AH. Progroups including linkages capable of metabolizing in vivo to yield such an intermediate group are well-known, and include, by way of example and not limitation, ethers, thioethers, silylethers, silylthioethers, esters, thioesters, carbonates, thiocarbonates, carbamates, thiocarbamates, ureas, thioureas, carboxamides, etc. In some instances, a “precursor” group that is oxidized by oxidative enzymes such as, for example, cytochrome P450 of the liver, to a metabolizable group, can be selected.

The identity of the R³ group can also be selected so as to impart the prodrug with desirable characteristics. For example, lipophilic groups can be used to decrease water solubility and hydrophilic groups can be used to increase water solubility. In this way, prodrugs specifically tailored for selected modes of administration can be obtained. The R³ group can also be designed to impart the prodrug with other properties, such as, for example, improved passive intestinal absorption, improved transport-mediated intestinal absorption, protection against fast metabolism (slow-release prodrugs), tissue-selective delivery, passive enrichment in target tissues, targeting-specific transporters, etc. Groups capable of imparting prodrugs with these characteristics are well-known, and are described, for example, in Ettmayer et al., 2004, J. Med. Chem. 47(10:2393-2404), the disclosure of which is incorporated by reference. All of the various groups described in these references can be utilized in the prodrugs described herein.

In some embodiments, R³ is —R^{f}, —C(O)R^{f}, —C(O)NR^{f}R^{f}, or —SiR^{f}R^{f}R^{f}, where the R^{f} groups are selected so as to impart the prodrugs with desired bioavailability, cleavage and/or targeting properties. In a specific embodiment, the R^{f} groups are selected to impart the prodrug with higher water-solubility than the underlying active 2,4-pyrimidinediamine drug. Thus, in some embodiments, the R^{f} groups are selected such that they, taken together with the heteroatom or group to which they are bonded, are hydrophilic in character. Such hydrophilic groups can be charged or uncharged, as is well-known in the art. As specific examples, each R^{f} group is independently hydrogen, optionally substituted lower alkyl, optionally substituted lower heteroalkyl, optionally substituted lower cycloalkyl, optionally substituted lower heterocycloalkyl, optionally substituted (C₆-C₁₀) aryl, optionally substituted 5-10 membered heteroaryl, optionally substituted (C₇-C₁₈) arylalkyl, or optionally substituted 6-18 membered heteroarylalkyl. The nature of any present substituents can vary widely, as is known in the art. In some embodiments any present substituents are, independently of one another, selected from R^{b}, defined above.

In a specific embodiment of the prodrugs of formula (I) and/or (Ia), (Ib), and (Ic) each R^{p} is independently R^{p2}, wherein R^{p2} is —C(R^{d}R^{d})_{y}-A-R³, wherein
each R^{d} is independently hydrogen, cyano, —C(O)R^{e1}, —C(O)OR^{e1}, —C(O)NR^{e1}R^{e1}, —C(OR^{e1})(OR^{e1}), optionally substituted (C₁-C₂₀) alkyl, (C₁-C₂₀) perfluoroalkyl, optionally substituted (C₇-C₃₀) arylalkyl, or optionally substituted 6-30 membered heteroarylalkyl, wherein
each R^{e1} is independently hydrogen, alkyl, aryl, arylalkyl, heteroaryl, or heteroarylalkyl;
y is 1, 2, or 3;
   A is O, S or NR⁵⁰, wherein R⁵⁰ is R^{d} or cycloalkyl; and
R³ is —R^{f}, —C(O)R^{f}, —C(O)O—R^{f}, —C(O)NR^{f}R^{f}, —Si(R^{f})₃, —P(O)(OH)₂, —P(O)(OH)(OR^{e}), —P(O)(OR^{e})₂, —P(OH)₂, —P(OH)(OR^{e}), or —P(OR^{e})₂, wherein
each R^{e} is independently (i) substituted or unsubstituted lower alkyl, substituted or unsubstituted (C₆-C₁₄) aryl, or substituted or unsubstituted (C₇-C₂₀) arylalkyl wherein each is optionally substituted with one or more groups (e.g., 1, 2, or 3 groups) independently selected from R^{b}, or (ii) —(CR^{d}R^{d})_{y}—OR^{f}, —(CR^{d}R^{d})_{y}—O—C(O)R^{f}, —(CR^{d}R^{d})_{y}—O—C(O)OR^{f}, —(CR^{d}R^{d})_{y}—S—C(O)R^{f}, —(CR^{d}R^{d})_{y}—S—C(O)OR^{f}, —(CR^{d}R^{d})_{y}—NH—C(O)R^{f}, —(CR^{d}R^{d})_{y}—NH—C(O)OR^{f}, or —Si(R^{f})₃;
or two R^{e} taken together with the oxygen atoms to which they are attached, form a 5-8 membered heterocycloalkyl group optionally substituted with substituted or unsubstituted lower alkyl, substituted or unsubstituted lower heterocycloalkyl, substituted or unsubstituted (C₆-C₁₄) aryl, substituted or unsubstituted (C₇-C₂₀) arylalkyl, or substituted or unsubstituted 5-14 membered heteroaryl wherein each is optionally substituted with one or more groups (e.g., 1, 2, or 3 groups) independently selected from R^{b};
each R^{f} group is independently hydrogen, optionally substituted lower alkyl, optionally substituted lower heteroalkyl, optionally substituted lower cycloalkyl, optionally substituted lower heterocycloalkyl, optionally substituted (C₆-C₁₀) aryl, optionally substituted 5-10 membered heteroaryl, optionally substituted (C₇-C₁₈) arylalkyl, or optionally substituted 6-18 membered heteroarylalkyl, wherein each is optionally substituted with one or more groups (e.g., 1, 2, or 3 groups) independently selected from R^{b},
or R⁵⁰ and R³ taken together with nitrogen atom to which they are both attached, form a three- to seven-membered ring,
where R^{b} is as defined for formula (I).

In another specific embodiment, of the prodrugs of formula (I) and/or (Ia), (Ib), and (Ic) each R^{p} is independently R^{p2}, wherein R^{p2} is —C(R^{d}R^{d})_{y}-A-R³, wherein
each R^{d} is independently hydrogen, cyano, —C(O)R^{e1}, —C(O)OR^{e1}, —C(O)NR^{e1}R^{e1}, —C(OR^{e1})(OR^{e1}), optionally substituted (C₁-C₂₀) alkyl, (C₁-C₂₀) perfluoroalkyl, optionally substituted (C₇-C₃₀) arylalkyl, or optionally substituted 6-30 membered heteroarylalkyl, wherein
each R^{e1} is independently hydrogen, alkyl, aryl, arylalkyl, heteroaryl, or heteroarylalkyl;
y is 1, 2, or 3;
   A is O, S or NR⁵⁰, wherein R⁵⁰ is R^{d} or cycloalkyl; and
R³ is —P(O)(OH)₂, —P(O)(OH)(OR^{e}), —P(O)(OR^{e})₂, —P(OH)₂, —P(OH)(OR^{e}), or —P(OR^{e})₂, wherein
each R^{e} is independently (i) substituted or unsubstituted lower alkyl, substituted or unsubstituted (C₆-C₁₄) aryl, or substituted or unsubstituted (C₇-C₂₀) arylalkyl wherein each is optionally substituted with one or more groups (e.g., 1, 2, or 3 groups) independently selected from R^{b}, or (ii) —(CR^{d}R^{d})_{y}—OR^{f}, —(CR^{d}R^{d})_{y}—O—C(O)R^{f}, —(CR^{d}R^{d})_{y}—O—C(O)OR^{f}, —(CR^{d}R^{d})_{y}—S—C(O)R^{f}, —(CR^{d}R^{d})_{y}—S—C(O)OR^{f}, —(CR^{d}R^{d})_{y}—NH—C(O)R^{f}, —(CR^{d}R^{d})_{y}—NH—C(O)OR^{f}, or —Si(R^{f})₃;
or two R^{e} taken together with the oxygen atoms to which they are attached, form a 5-8 membered heterocycloalkyl group optionally substituted with substituted or unsubstituted lower alkyl, substituted or unsubstituted lower heterocycloalkyl, substituted or unsubstituted (C₆-C₁₄) aryl, substituted or unsubstituted (C₇-C₂₀) arylalkyl, or substituted or unsubstituted 5-14 membered heteroaryl wherein each is optionally substituted with one or more groups (e.g., 1, 2, or 3 groups) independently selected from R^{b};
each R^{f} group is independently hydrogen, optionally substituted lower alkyl, optionally substituted lower heteroalkyl, optionally substituted lower cycloalkyl, optionally substituted lower heterocycloalkyl, optionally substituted (C₆-C₁₀) aryl, optionally substituted 5-10 membered heteroaryl, optionally substituted (C₇-C₁₈) arylalkyl, or optionally substituted 6-18 membered heteroarylalkyl, wherein each is optionally substituted with one or more groups (e.g., 1, 2, or 3 groups) independently selected from R^{b},
or R⁵⁰ and R³ taken together with nitrogen atom to which they are both attached, form a three- to seven-membered ring,
where R^{b} is as defined for formula (I).

In a specific embodiment, the progroups on the prodrugs of formula (I) and/or (Ia), (Ib), and (Ic) are of the formula —CR^{d}R^{d}-A-R³, where R³ is —(CH₂)ᵢ—R^{b}, —C(O)R^{a}, —C(O)—(CH₂)ᵢ—R^{b}, —C(O)O—R^{a}, or —C(O)O—(CH₂)ᵢ—R^{b}, where X, R^{a}, R^{b} and R^{d} are as previously defined, and i is 0, 1, 2, 3, 4, 5, or 6. Specific, non-limiting, examples of exemplary water-solubility increasing progroups include, by the way of example and not limitation, hydrophilic groups such as alkyl, aryl, arylalkyl, or heterocycloalkyl groups substituted with one or more of an amine, alcohol, a carboxylic acid, a phosphorous acid, a sulfoxide, a sugar, an amino acid, a thiol, a polyol, a ether, a thioether and a quaternary amine salt.

One important class of progroups includes progroups that contain a phosphate group, for example, phosphate-containing progroups of the formula —(CR^{d}R^{d})_{y}—O—P(O)(OH)₂, where R^{d} is as defined above and y is 1, 2, or 3, typically 1 or 2. In a specific embodiment, each R^{d} is independently hydrogen, substituted or unsubstituted lower alkyl, substituted or unsubstituted (C₆-C₁₄) aryl or substituted or unsubstituted (C₇-C₂₀) arylalkyl.

While not intending to be bound by any theory of operation, it is believed that such phosphate-containing progroups R^{p} act as substrates for both alkaline and acid phosphatase enzymes, leading to their removal from the prodrugs under physiological conditions of use. As alkaline phosphatases are abundant in the digestive tract of humans, phosphate-containing progroups R^{p} that can be cleaved in the presence of alkaline phosphatases are particularly suitable for formulating phosphate-containing prodrugs intended for oral administration. Specific examples of phosphate-containing progroups R^{p} suitable for use in prodrugs intended for oral administration include, but are not limited to, groups of the formula —(CR^{d}R^{d})_{y}—P(O)(OH)₂ in which each R^{d} is independently hydrogen or unsubstituted lower alkanyl. Exemplary embodiments of such phosphate-containing progroups include, but are not limited to, —CH₂—O—P(O)(OH)₂ and —CH₂CH₂—O—P(O)(OH)₂.

Although phosphate-containing prodrugs suitable for oral administration are of interest, skilled artisans will appreciate that prodrugs including phosphate-containing progroups R^{p} can be administered via other routes of administration, as phosphatases are distributed throughout the body. For example, exemplary prodrug Compound P-1 has been found to metabolize to the active drug Compound 1 in in vitro experiments carried out with rat plasma, as well as with rat hepatic and intestinal microsomal preparations, indicating that phosphatases are also present in plasma. Thus, the only requirement is that the particular phosphate-containing progroup R^{p} selected should be removable under the conditions of intended use.

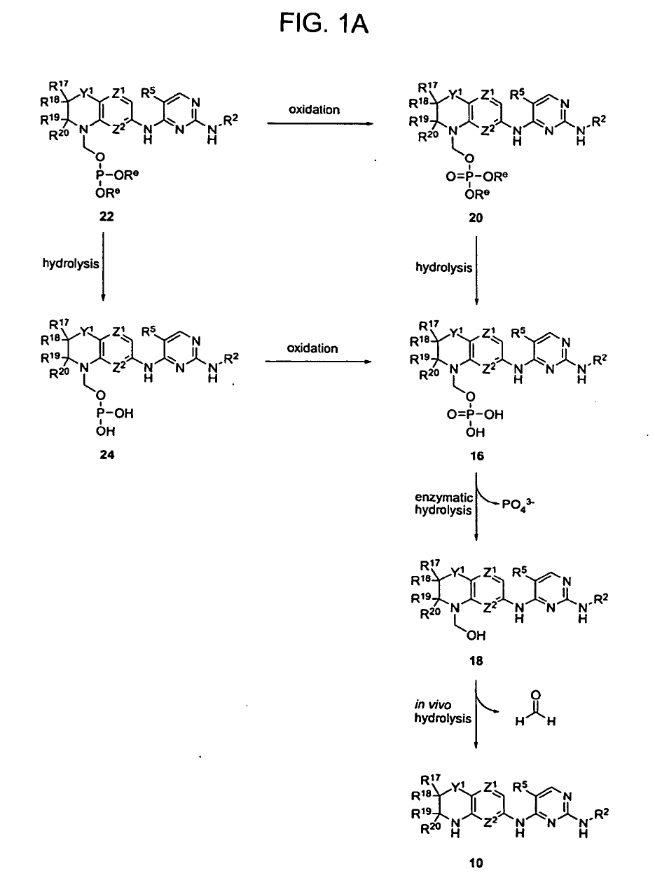

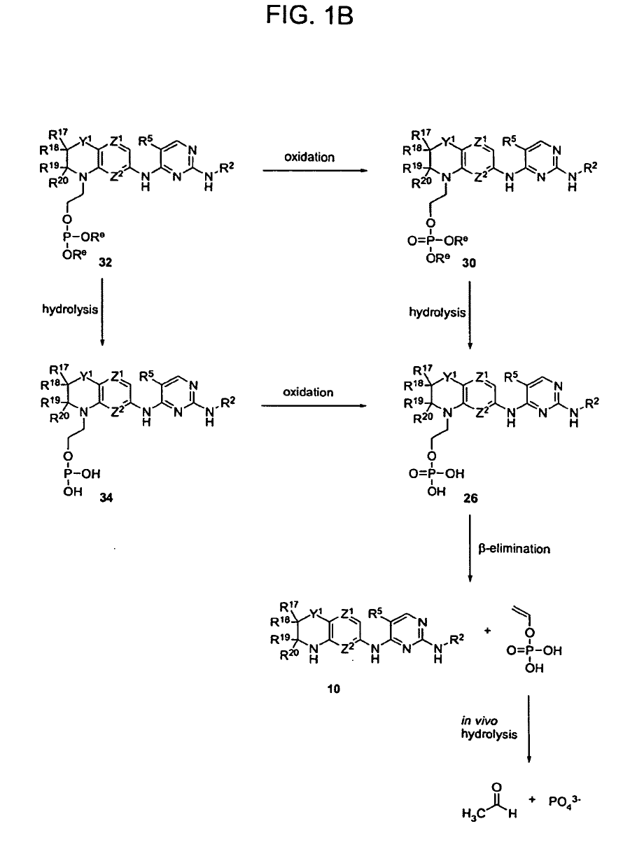

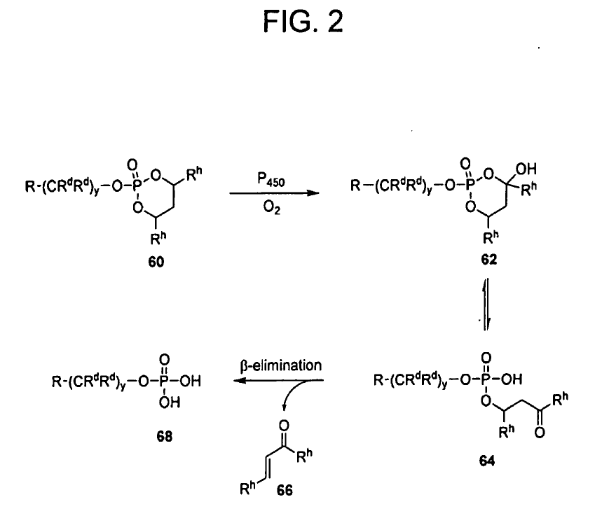

While not intending to be bound by any theory of operation, it is believed that when y is 1, phosphate-containing prodrugs, such as those according to structural formula (Ia), are metabolized to the active 2,4-pyrimidinediamine compound via the corresponding hydroxymethylamine. This metabolism is illustrated in FIG. 1A. Referring to FIG. 1A, removal of phosphoric acid from phosphate prodrug 16 via enzymatic hydrolysis yields the corresponding hydroxymethylamine 18, which undergoes hydrolysis in vivo to yield formaldehyde and active 2,4-pyrimidinediamine compound 10.

Referring to FIG. 1B, when y is 2, it is believed that in vivo hydrolysis of phosphate prodrug 26 yields active 2,4-pyrimidinediamine 10 and enol phosphate, which then hydrolyses in vivo to acetaldehyde and phosphoric acid.

Referring again to FIG. 1A, skilled artisan will appreciate that while hydroxymethylamine 18 metabolizes under physiological conditions to yield active 2,4-pyrimidinediamine compound 10, it is stable at pH 7 and can therefore be prepared and administered as a hydroxyalkyl-containing prodrug of active compound 10. Thus, in some embodiments of the prodrugs of structural formula (I), R^{p} is a hydroxyalkyl-containing progroup of the formula —CR^{d}R^{d}—OH, where R^{d} is as previously defined. In a specific exemplary embodiment, R^{p} is —CH₂OH.

Still referring again to FIG. 1A, skilled artisans will also appreciate that phosphate prodrugs can be generated by in vivo hydrolysis of phosphate ester prodrugs, such as phosphate ester prodrugs 20 and/or by in vivo oxidation of phosphite prodrugs, such as phosphite prodrugs 24. Such phosphate ester and phosphite prodrugs can in turn be generated by either in vivo oxidation or hydrolysis of phosphite ester prodrugs such as phosphite ester prodrugs 22. The corresponding phosphate ester, phosphite and phosphite ester prodrugs of phosphate prodrug 26 are illustrated in FIG. 1B as compounds 30, 34 and 32, respectively. Thus, as will be appreciated by skilled artisans, prodrugs that include precursors of phosphates that can metabolize into phosphate groups in vivo are also included in the present invention.

In some embodiments of such prodrugs, the phosphorous-containing progroup R^{p} comprises a phosphite group. A specific exemplary embodiment of such phosphite-containing prodrugs includes prodrug compounds in which the progroup R^{p} is of the formula —(CR^{d}R^{d}))_{y}—O—P(OH)(OH), where R^{d} and y are as previously defined.

In other embodiments of such prodrugs, the phosphorous-containing progroup R^{p} comprises an acyclic phosphate ester or phosphite ester group. Specific exemplary embodiments of such acyclic phosphate ester and phosphite ester prodrugs include progroups R^{p} of the formula —(CR^{d}R^{d})_{y}—O—P(O)(OH)(OR^{e}), —(CR^{d}R^{d})_{y}—O—P(O)(OR^{e})₂, —(CR^{d}R^{d})_{y}—O—P(OH)(OR^{e}) and —(CR^{d}R^{d})_{y}—O—P(OR^{e})₂, where each R^{e} is independently substituted or unsubstituted lower alkyl, substituted or unsubstituted (C₆-C₁₄) aryl (e.g., phenyl, naphthyl, 4-lower alkoxyphenyl, 4-methoxyphenyl), substituted or unsubstituted (C₇-C₂₀) arylalkyl (e.g., benzyl, 1-phenylethan-1-yl, 2-phenylethan-1-yl), —(CR^{d}R^{d})_{y}—OR^{f}, —(CR^{d}R^{d})_{y}—O—(O)R^{f}, —(CR^{d}R^{d})_{y}—O—C(O)OR^{f}, —(CR^{d}R^{d})_{y}—S—C(O)R^{f}, (CR^{d}R^{d})_{y}—S—C(O)OR^{f}, (CR^{d}R^{d})_{y}—NH—C(O)R^{f}, —(CR^{d}R^{d})_{y}—NH—C(O)OR^{f}, or —Si(R^{f})₃, wherein each R^{f} is independently hydrogen, unsubstituted or substituted lower alkyl, substituted or unsubstituted (C₆-C₁₄) aryl, or substituted or unsubstituted (C₇-C₂₀) arylalkyl, and R^{d} and y are as previously defined.

In still other embodiments, phosphorous-containing prodrugs that include phosphate precursors are prodrugs in which the phosphorous-containing progroup R^{p} comprises a cyclic phosphate ester of the formula (XX),

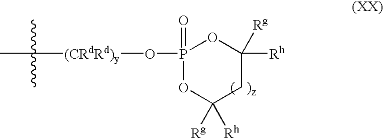

where each R^{g} is independently hydrogen or lower alkyl; each R^{h} is independently hydrogen, substituted or unsubstituted lower alkyl, substituted or unsubstituted lower heterocycloalkyl, substituted or unsubstituted (C₆-C₁₄) aryl, substituted or unsubstituted (C₇-C₂₀) arylalkyl, or substituted or unsubstituted 5-14 membered heteroaryl; z is 0, 1, or 2; and R^{d} and y are as previously defined.

In still other embodiments, phosphorous-containing prodrugs that include phosphate precursors are prodrugs in which the phosphorous-containing progroup R^{p} comprises a cyclic phosphite ester of the formula (XXI),

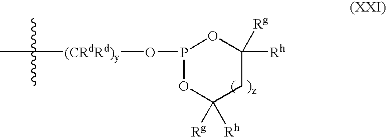

where R^{g}, R^{h}, R^{d}, y and z are as previously defined.

In some embodiments, the substituents R^{h} on such cyclic phosphate ester and phosphite ester prodrugs are selected such that the progroup is metabolized in vitro by esterase enzymes. Specific examples of such phosphate ester and phosphite ester progroups include those in which each R^{h} is independently hydrogen, lower alkyl, methyl, ethyl or propyl.

In some embodiments of formulae (XX) and (XXI) each R^{p} is independently of any one of formulae (XXIII)-(XXXVIII):

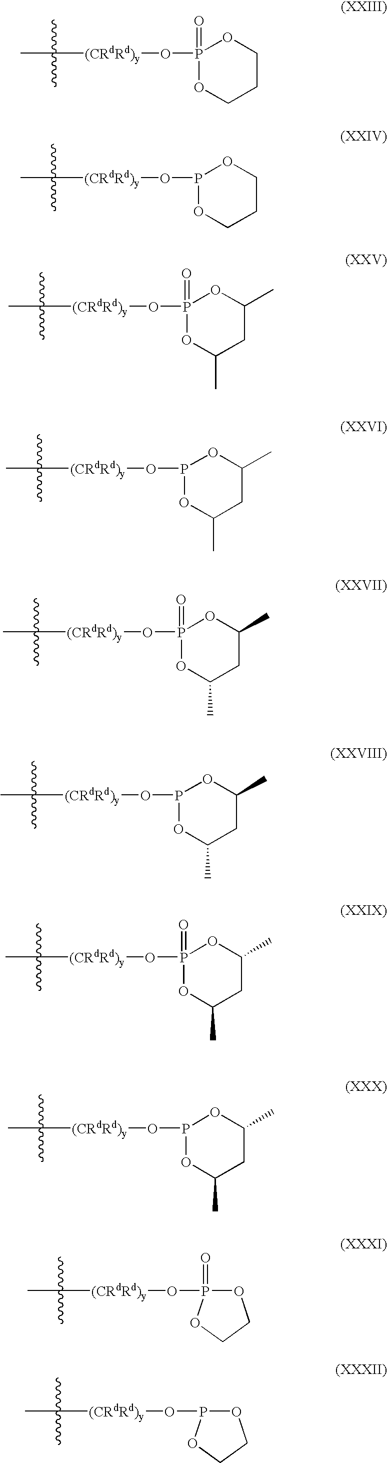

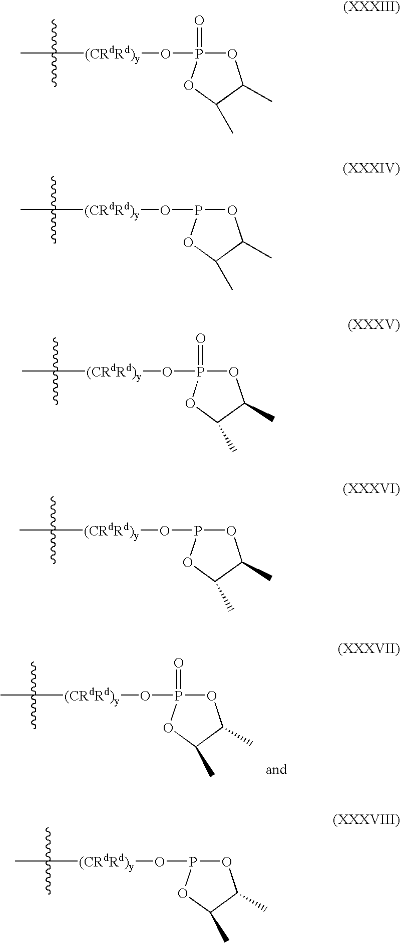

wherein R^{d} and y are defined previously.

Many of these phosphate esters and phosphite esters are acid label and, when administered orally, metabolize to the corresponding phosphates and phosphites under the acidic conditions of the stomach and/or gut.

Thus, in the phosphorous-containing prodrugs described herein, the identity of the particular phosphorous-containing progroups R^{p} employed can be selected to tailor the prodrugs for particular modes of delivery, etc.

The suitability of any particular progroup R^{p} for a desired mode of administration can be confirmed in biochemical assays. For example, if a prodrug is to be administered by injection into a particular tissue or organ, and the identities of the various phosphatases expressed in the tissue or organ are known, the particular prodrug can be tested for metabolism in biochemical assays with the isolated phosphatase(s). Alternatively, the particular prodrug can be tested for metabolism to an active 2,4-pyrimidinediamine compound with tissue and/or organ extracts. Using tissue and/or organ extracts can be of particular convenience when the identity(ies) of the phosphatases expressed in the target tissues or organs are unknown, or in instances when the isolated phosphatases are not conveniently available. Skilled artisans will be able to readily select progroups R^{p} having metabolic properties (such as kinetics) suitable for particular applications using such in vitro tests. Of course, specific prodrugs could also be tested for suitable metabolism in in vitro animal models.

In some embodiments, the prodrugs are prodrugs according to structural formula (I), (Ia), (Ib), or (Ic) that have one or more features selected from:
(i) R⁵ is fluoro;
(ii) R² is a phenyl optionally substituted with one or more of the same or different R⁸ groups;
(iii) R² is 3,4,5-tri(loweralkoxy)phenyl;
(iv) R² is 3,4,5-trimethoxyphenyl;
(v) Y or Y¹ is O; Z¹ is CH, Z² is N; R¹⁷ and R¹⁸ are each methyl; and R¹⁹ and R²⁰ are taken together to form an oxogroup; and
(vi) R^{p} is a hydroxyalkyl-containing progroup of the formula —CH₂OH, or a phosphate-containing progroup of the formula —(CR^{d}R^{d})_{y}—O—P(O)(OH)₂, or a phosphate ester, phosphite or phosphite ester analog thereof, wherein y is 1 or 2 and each R^{d} is independently hydrogen or unsubstituted lower alkyl, or
(vii) R^{p} is —CH₂OH, —CH₂SH, —CH₂NH₂, —CH₂NHR⁵⁰, —CH₂N(R⁵⁰)₂, —CH₂-A-R^{f}, —CH₂-A-C(O)R^{f}, —CH₂-A-C(O)OR^{f}, or —CH₂-A-C(O)NR^{f}R^{f}, where A, R⁵⁰ and R^{f} are as previously defined.

In some embodiments, the prodrugs of structural formulae (I), (Ia), (Ib), and (Ic) have two or three of the above-delineated features. In one specific embodiment, the prodrugs have features (i), (iii) and (v). In another specific embodiment, the prodrugs have features (i), (iv) and (v). In still another specific embodiment, the prodrugs have features (i), (iii), (v) and (vi) or (vii). In still another specific embodiment, the prodrugs have features (i), (iv), (v) and (vi) or (vii). In still another specific embodiment, R^{p} is a phosphate-containing progroup of the formula —(CR^{d}R^{d})_{y}—O—P(O)(OH)₂, where R^{d} and y are as defined above.

The invention comprises a produg salt of the compounds described above, or a hydrate, solvate or N-oxide thereof, comprising a 2,4-pyrimidineamine moiety and at least one progroup salt R^{q} linked covalently to a primary or secondary amino nitrogen atom of the 2,4-pyrimidinediamine moiety. The progroup salt R^{q} may be any suitable salt of any acidic progroup R^{p}, as discussed above. For example, the progroup salt R^{q} may be a salt of a phosphate-containing progroup, a carbonate-containing progroup, or a sulfonate-containing progroup. The counterion may be, for example, an alkali cation (e.g., Na⁺), an alkaline earth cation (e.g. [Ca²⁺]_{0.5}), or an ammonium cation (e.g., NH₄⁺). Examples of suitable progroup salts R^{q} include: —(CR^{d}R^{d})_{y}-A-P(O)(O⁻)₂M⁺₂, —(CR^{d}R^{d})_{y}-A—P(O)(OR^{e})(O⁻)M⁺, —(CR^{d}R^{d})_{y}-A-P(O)(OH)(O⁻)M⁺, —(CR^{d}R^{d})_{y}—P(O)₂, —(CR^{d}R^{d})_{y}-A-P(OR^{e})(O⁻)M⁺, —(CR^{d}R^{d})_{y}-A-P(OH)(O⁻)M⁺ an (CR^{d}R^{d})_{y}-A-COO⁻M⁺, wherein A, R^{d}, R^{e}, and y are as described above and M⁺ is alkali cation, and alkaline earth cation, or an ammonium cation (e.g., a lysine cation or an arginine cation). In one embodiment of the invention, A is O.

In another example, R^{q} is —CH₂—O—P(O)(O⁻)₂Na₂; —CH₂—O—P(O)(OH)(O⁻)Na⁺; —CH₂—O—P(O)(O⁻)₂K⁺₂; —CH₂—O—P(O)(OH)(O⁻)K⁺; —CH₂—O—P(O)(O⁻)₂Ca²⁺; —CH₂—O—P(O)(OH)(O⁻) [Ca²⁺]_{0.5}; —CH₂—O—P(O)(O⁻)₂Mg²⁺; or —CH₂—O—P(O)(OH)(O⁻)[Mg²⁺]_{0.5}. In certain embodiments of the invention, M⁺ is an alkali cation, an amino acid cation, or an ammonium cation. In certain embodiments of the invention, the prodrug salts are in solid or semi-solid form, and are not dissolved in aqueous solution.

Another aspect of the invention relates to hydrates of the prodrug salts described above. Hydrates desirably have, for example, ranging from about 1 to about 15 moles of water per mole of prodrug salt. As is described in more detail below, the inventors have determined that prodrug salt hydrates of the present invention have surprisingly desirable stability characteristics, remaining at a stable level of hydration over wide ranges of relative humidities. The inventors surmise that the hydration stability characteristics are due to the high binding energy of the water molecules to the hydrate as well as the special stability of the three dimensional order of the crystalline matrix and its inability to spatially rearrange itself in response to dehydration.

According to one aspect of the invention, prodrug salt hydrates of the invention contain from about 3% to about 17% by weight of water, more preferably from about 13.0% to 16.5% by weight of water. In one embodiment, prodrug salt hydrates of the present invention have the formula

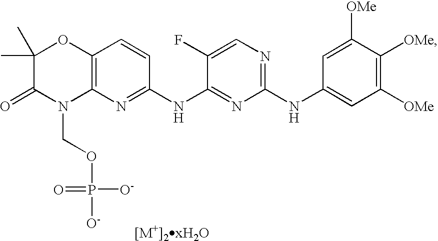

in which x is from about 1 to about 15 and M⁺ is as defined above. More preferably x is from about 5 to about 10. In one particular embodiment, x is from about 5 to about 8. For example, in one embodiment the prodrug salt hydrate of the present invention has the structure:

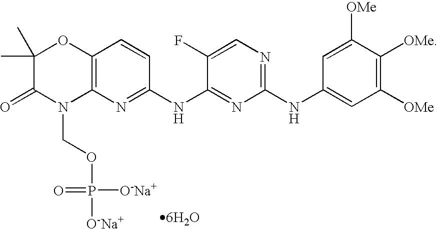

Control of pH in the formation of the salts is desirable. Desirable pH values for the formation of di-M⁺ salts of compound 4 range from about 8 to about 11, more preferably from about 9 to about 11, even more preferably from about 9.3 to about 10.5. For example, desirable pH values for the formation of the disodium salt of compound 4 fall within these ranges. Desirable pH values for the formation of mono-M⁺ (e.g., monosodium) salts of compound 4 range from about 5 to about 7, more preferably from about 5 to about 6, even more preferably from about 5.0 to about 5.5.

According to the fifth aspect, the compounds useful in the methods of the invention are 2,4-pyrimidinediamine compounds according to structural formulae (II) and (III):

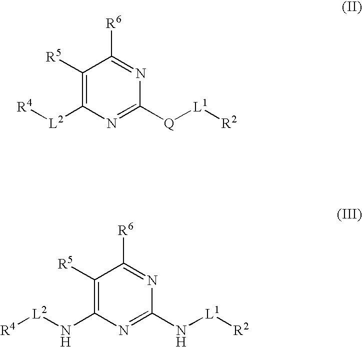

including pharmaceutically acceptable salts, hydrates, solvates and N-oxides thereof, wherein:
Q is O or S;
L¹ is a direct bond or a linker;
L² is a direct bond, —NH—, —O—, —S—, or a linker;
R² is (C₁-C₆) alkyl optionally substituted with one or more of the same or different R⁵ groups, (C₃-C₈) cycloalkyl optionally substituted with one or more of the same or different R⁸ groups, cyclohexyl optionally substituted with one or more of the same or different R⁸ groups, 3-8 membered heterocycloalkyl optionally substituted with one or more of the same or different R⁸ groups, (C₅-C₁₅) aryl optionally substituted with one or more of the same or different R⁸ groups, phenyl optionally substituted with one or more of the same or different R⁵ groups, or 5-15 membered heteroaryl optionally substituted with one or more of the same or different R⁸ groups;
R⁴ is hydrogen, (C₁-C₆) alkyl optionally substituted with one or more of the same or different R⁸ groups, (C₃-C₈) cycloalkyl optionally substituted with one or more of the same or different R⁸ groups, cyclohexyl optionally substituted with one or more of the same or different R⁸ groups, 4-10 membered bridged cycloalkyl optionally substituted with one or more of the same or different R⁸ groups, 3-8 membered heterocycloalkyl optionally substituted with one or more of the same or different R⁸ groups, (C₅-C₁₅) aryl optionally substituted with one or more of the same or different R⁸ groups, phenyl optionally substituted with one or more of the same or different R⁸ groups, or 5-15 membered heteroaryl optionally substituted with one or more of the same or different R⁸ groups;
R⁵ is R⁶, (C₁-C₆) alkyl optionally substituted with one or more of the same or different R⁸ groups, (C₁-C₄) alkanyl optionally substituted with one or more of the same or different R⁸ groups, (C₂-C₄) alkenyl optionally substituted with one or more of the same or different R⁸ groups, or (C₂-C₄) alkynyl optionally substituted with one or more of the same or different R⁸ groups;
each R⁶ is independently hydrogen, an electronegative group, —OR^{d}, —SR^{d}, (C₁-C₃) haloalkyloxy, (C₁-C₃) perhaloalkyloxy, —NR^{c}R^{c}, halogen, (C—C) haloalkyl, (C₁-C₃) perhaloalkyl, —CF₃, —CH₂CF₃, —CF₂CF₃, —CN, —NC, —OCN, —SCN, —NO, —NO₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)NR^{c}R^{c}, —OS(O)₂NR^{c}R^{c}, —C(O)R^{d}, —C(O)OR^{d} —C(O)NR^{c}R^{c}, —C(NH)NR^{c}R^{c}, —OC(O)R^{d}, —SC(O)R^{d}, OC(O)OR^{d}, —SC(O)OR^{d} —OC(O)NR^{c}R^{c}, —SC(O)NR^{c}R^{c}, —OC(NH)NR^{c}R^{c}, —SC(NH)NR^{c}R^{c}, —[NHC(O)]ₙR^{d}, —[NHC(O)]ₙOR^{d}, —[NHC(O)]ₙNR^{c}R^{c}, —[NHC(NH)]ₙNR^{c}R^{c}, (C₅-C₁₀) aryl optionally substituted with one or more of the same or different R⁸ groups, phenyl optionally substituted with one or more of the same or different R⁸ groups, (C₆-C₁₆) arylalkyl optionally substituted with one or more of the same or different R⁸ groups, 5-10 membered heteroaryl optionally substituted with one or more of the same or different R⁸ groups, or 6-16 membered heteroarylalkyl optionally substituted with one or more of the same or different R⁵ groups;
R⁸ is R^{a}, R^{b}, —B(OR^{a})₂, —B(NR^{c}R^{c})₂, —(CH₂)ₘ—R^{b}, —(CHR^{a})ₘ—R^{b}, —O(CH₂)ₘ R^{b}, —S—(CH₂)ₘ—R^{b}, —O—CHR^{a}R^{b}, —O—CR^{a} (R^{b})₂, —O—(CHR^{a})ₘ—R^{b}, —O—(CH₂)ₘ—CH[(CH₂)ₘ—R^{b}]R^{b}, —S—(CHR^{a})ₘ—R^{b}, —C(O)NH—(CH₂)ₘ—R^{b}, —C(O)NH—(CHR^{a})ₘ—R^{b}, —O—(CH₂)ₘ—C(O)NH—(CH₂)R^{b}, —S—(CH₂)ₘ—C(O)NH—(CH₂)ₘ—R^{b}, —O— (CHR^{a})ₘ—R^{b}, —O—(CH₂)ₘ—C(O)NH—(CH₂)ₘ—R^{b}, —S—(CHR^{a})ₘ—C(O)NH—(CHR^{a})ₘ—R^{b}, —NH—(CH₂)ₘ—R^{b}, —NH—(CHR^{a})ₘ—R^{b}, —NH[(CH₂)ₘ—R^{b}], —N[(CH₂)ₘR^{b}]₂, —NH—C(O)—NH—(CH₂)ₘ—R^{b}, —NH—C(O)—(CH₂)ₘ—CHR^{b}R^{b}, —NH—(CH₂)ₘ—C(O)—NH—(CH₂)ₘ—R^{b}, R^{a} substituted with one or more of the same or different R^{a} or R^{b}; or —OR^{a} substituted with one or more of the same or different R^{a} or R^{b};
each R^{a} is independently hydrogen, (C₁-C₆) alkyl, (C₃-C₈) cycloalkyl, cyclohexyl, (C₄-C₁₁) cycloalkylalkyl, (C₅-C₁₀); aryl, phenyl, (C₆-C₁₆) arylalkyl, benzyl, 2-6 membered heteroalkyl, 3-8 membered heterocycloalkyl, morpholinyl, piperazinyl, homopiperazinyl, piperidinyl, 4-11 membered heterocycloalkylalkyl, 5-10 membered heteroaryl, or 6-16 membered heteroarylalkyl, wherein each cycloalkyl, aryl, heterocycloalkyl, and heteroaryl group or part of a larger group is optionally substituted with one or more groups which are independently (C₁-C₃) alkyl, cyclopropyl, halogen, amino, hydroxy, carboxy, carbamoyl, cyano, nitro, or trifluoromethyl;
each R^{b} is ═O, —OR^{d}, (C₁-C₃) haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d} OC(O)OR^{d}, —OC(O)NR^{c}R^{c}, —OC(NH)NR^{c}R^{c}, —OC(Na)NR^{c}R^{c}, —[NHC(O)]R^{d}, —[NR^{a}C(O)]R^{d}, —[NHC(O)]OR^{d}, —[NR^{a}C(O)], —OR^{d}, [NHC(O)]ₙNR^{c}R^{c}, —[NR^{a}C(O)]ₙNR^{c}R^{c}, —[NHC(NH)]ₙNR^{c}R^{c}, or —[NR^{c}C(NR^{a})]ₙNR^{c}R^{c};
each R^{c} is independently R^{a};
or two R^{c} is taken together with the nitrogen atom to which they are each bonded form a 5 to 8-membered heterocycloalkyl or heteroaryl having one or more of the same or different additional heteroatoms and which is optionally substituted with one or more of the same or different R^{a} or suitable R^{b} groups;
each R^{d} is independently R^{a};
each m is independently 1, 2, or 3; and
each n is independently 0, 1, 2, or 3.

In the compounds of structural formulae (II) and (III), L¹ and L² are independently a direct bond or a linker. Thus, as will be appreciated by skilled artisans, the substituents R² and/or R⁴ may be bonded either directly to their respective nitrogen atoms or, alternatively, spaced away from their respective nitrogen atoms by way of a linker. The identity of the linker is not critical and typical suitable linkers include, but are not limited to, (C₁-C₆) alkyldiyls, (C₁-C₆) alkanos and (C₁-C₆) heteroalkyldiyls, each of which may be optionally substituted with one or more of the same or different R⁸ groups, where R⁸ is as previously defined for structural formula (II) and (III).

In a specific embodiment of the compounds of structural formulae (II) and (III), L¹ and L² are each independently a direct bond, (C₁-C₃) alkyldiyl optionally substituted with one or more of the same or different R^{a}, R^{b}, or R⁹ groups and 1-3 membered heteroalkyldiyl optionally substituted with one or more of the same or different R^{a}, R^{b}, or R⁹ groups, wherein each R⁹ is independently (C₁-C₃) alkyl, —OR^{a}, —C(O)OR^{a}, (C₅-C₁₀) aryl optionally substituted with one or more of the same or different halogens, phenyl optionally substituted with one or more of the same or different halogens, 5-10 membered heteroaryl optionally substituted with one or, more of the same or different halogens or 6 membered heteroaryl optionally substituted with one or more of the same or different halogens; and R^{a} and R^{b} are as previously defined for structural formula (II) and (III). Specific R⁹ groups that may be used to substitute L¹ and L² include —OR^{a}, —C(O)OR^{a}, phenyl, halophenyl and 4-halophenyl, wherein R^{a} is as previously defined for structural formula (II).

In another specific embodiment of the compounds of structural formulae (II) and (III), L¹ and L² are each independently methylene, ethylene or propylene, wherein L¹ and L² are optionally monosubstituted with an R⁹ group, where R⁹ is as previously defined above.

In all of the above embodiments, each R^{a} group in R⁹ are independently hydrogen, (C₁-C₆) alkyl, phenyl and benzyl.

In still another specific embodiment, L¹ and L² are each a direct bond such that the 2,4-pyrimidinediamine compounds of the invention are compounds according to structural formula (IIa) and (IIIa):

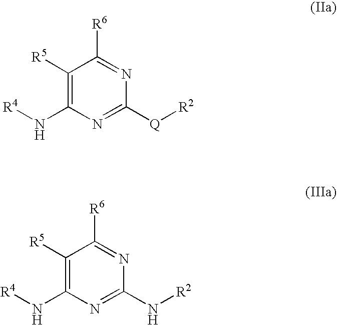

including pharmaceutically acceptable salts, hydrates, solvates and N-oxides thereof, wherein R², R⁴, R⁵ and R⁶ are as previously defined for structural formula (II) and (III). Additional specific embodiments of the 2,4-pyrimidinediamine compounds of the invention are described below.

In a first embodiment of the compounds of structural formulae (II), (III), (IIa) and (IIIa), R², R⁴, R⁵, R⁶, L¹ and L² are as previously defined for their respective structures (II), (III), (IIa) and (IIIa),

wherein R² is a phenyl group substituted with three R⁸ groups.

In a specific embodiment of this first embodiment of structural formulae (II), (III), (IIa) and (IIIa), R² is a trisubstituted phenyl having the formula (XC):

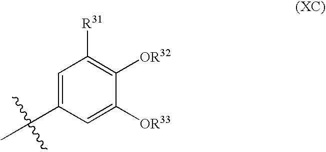

wherein R³¹ is hydrogen, halo, straight-chain or branched (C₁-C₆) alkyl optionally substituted with one or more of the same or different R²⁵ groups; hydroxyl; (C₁-C₆) alkoxy optionally substituted with one or more of the same or different phenyl or R²⁵ groups; thiol (—SH); (C₁-C₆) alkylthio optionally substituted with one or more of the same or different phenyl or R²⁵ groups; amino (—NH₂); —NHR²⁶, or —NR²⁶R²⁶;

R²² and R²³ are independently a (C₁-C₆) straight-chain or branched alkyl optionally substituted with one or more of the same or different R²⁵ groups;

R²⁵ is halo, hydroxyl, (C₁-C₆) alkoxy, thiol, (C₁-C₆) alkylthio, (C₁-C₆) alkylamino or (C₁-C₆) dialkylamino; and

each R²⁶ is independently a (C₁-C₆) alkyl optionally substituted with one or more of the same or different phenyl or R²⁵ groups or —C(O)R²⁷,

where R²⁷ is a (C₁-C₆) alkyl optionally substituted with one or more of the same or different phenyl or R²⁵ groups.

In another specific embodiment of this first embodiment of structural formulae (II), (III), (IIa) and (IIIa), R² is a trisubstituted phenyl of formula (XC), wherein R³¹ is methoxy optionally substituted with one or more of the same or different halo groups and/or R³² and R³³ are each, independently of one another, a methyl or ethyl optionally substituted with one or more of the same or different halo groups.

In another specific embodiment of the compounds of structural formulae (II), (III), (IIa) and (IIIa), R², R⁴, R⁵ and L² are as previously described for their respective structures (II), (III), (IIa) and (IIIa), L¹ is a direct bond and R⁶ is hydrogen.

In another specific embodiment of the compounds of structural formulae (II), (III), (IIa) and (IIIa), when R⁵ is cyano or —C(O)NHR, where R is hydrogen or (C₁-C₆) alkyl; and R⁶ is hydrogen, then R² is other than a substituted phenyl group.

In another specific embodiment, the compounds of structural formulae (III) and (IIa) exclude compounds in which R² and R⁴ are each independently a substituted or unsubstituted pyrrole or indole ring which is attached to the remainder of the molecule via its ring nitrogen atom.

In another specific embodiment of the compounds of structural formulae (II), (III), (IIa) and (IIa), R⁵ is fluoro.

Those of skill in the art will appreciate that in the compounds of formulae (II), (III), (IIa) and (IIa), R² and R⁴ may be the same or different, and can vary broadly. When R² and/or R⁴ are optionally substituted rings, such as optionally substituted cycloalkyls, heterocycloalkyls, aryls and heteroaryls, the ring can be attached to the remainder of the molecule through any available carbon or heteroatom. The optional substituents can be attached to any available carbon atoms and/or heteroatoms.

In a another specific embodiment of the compounds of structural formulae (II), (III), (IIa) and (IIa), R² and/or R⁴ is an optionally substituted phenyl or an optionally substituted (C₅-C₁₅) aryl, subject to the provisos that,
(1) when R⁶ is hydrogen, then R² is not 3,4,5-trimethoxyphenyl or 3,4,5-tri (C₁-C₆) alkoxyphenyl;
(2) when R² is a 3,4,5-trisubstituted phenyl, then the substituents at the 3- and 4-positions are not simultaneously methoxy or (C₁-C₆) alkoxy; and/or
(3) when R⁶ is hydrogen and R⁴ is (C₁-C₆) alkyl, (C₃-C₈) cycloalkyl, 3-8 membered heterocycloalkyl or 5-15 membered heteroaryl, then R⁵ is other than cyano.

The optionally substituted aryl or phenyl group can be attached to the remainder of the molecule through any available carbon atom. Specific examples of optionally substituted phenyls include phenyls that are optionally mono-, di- or tri-substituted with the same or different R⁸ groups, where R⁸ is as previously defined for structural formula (II) and subject to the above provisos. When the phenyl is mono-substituted, the R⁸ substituent can be positioned at either the ortho, meta or para position.

In one embodiment, when R² is phenyl mono-substituted with R⁸, then R⁸ is (C₁-C₁₀) alkyl, (C₁-C₁₀) branched alkyl, —OR^{a} optionally substituted with one or more of the same or different R^{b} groups, —O—C(O)OR^{a}, —O—(CH₂)ₘ, C(O)OR^{a}, —C(O)OR^{a}, —O—(CH₂)ₘ—NR^{c}R^{c}, —O—C(O)NR^{c}R^{c}, —O—(CH₂)ₘ—C(O)NR^{c}R^{c}, —O—C(NH)NR^{c}R^{c}, —O—(CH₂)ₘ—C(NH)NR^{c}R^{c}, or —NH—(CH₂)ₘ—N—R^{c}R^{c}, where m, R^{a} and R^{c} are as previously defined for structural formula (II) and (III). In particular embodiments of these compounds,
(a) —NR^{c}R^{c} is a 5-6 membered heteroaryl which optionally includes one or more of the same or different additional heteroatoms; for example, but not limited to, oxadiazolyl, triazolyl, thiazolyl, oxazolyl, tetrazolyl and isoxazolyl;
(b) —NR^{c}R^{c} is a 5-6 membered saturated heterocycloalkyl ring which optionally includes one or more of the same or different heteroatoms; for example, but not limited to, pyrrolidinyl, pyrazolidinyl, imidazolidinyl, piperidinyl, piperazinyl and morpholinyl;
(c) each R^{a} is independently a (C₁-C₆) alkyl and/or each —NR^{c}R^{c} is —NHR^{a}, where R^{a} is a (C₁-C₆) alkyl;
(d) R³ is —O—CH₂—C(O)NHCH₃; or
(e) R⁸ is —OH.

In another embodiment, when R² is di-substituted or tri-substituted phenyl, then the R⁸ substituents can be positioned at any combination of positions. For example, the R⁸ substituents may be positioned at the 2,3-, 2,4-, 2,5-, 2,6-, 3,4-, 3,5-, 2,3,4-, 2,3,5-, 2,3,6-, 2,4,5-, 2,4,6-, 2,5,6- or 3,4,5-positions. In one embodiment of compounds including a disubstituted phenyl, the substituents are positioned other than 3,4. In another embodiment they are positioned 3,4. In one embodiment of compounds including a trisubstituted phenyl, the substituents are positioned other than 3,4,5 or, alternatively, no two of the substituents are positioned 3,4. In another embodiment, the substituents are positioned 3,4,5. Specific examples of R⁸ substituents in such di- and trisubstituted phenyls include the various R⁸ substituents described above in connection with the ortho, meta and para substituted phenyls.

In another specific embodiment, when R² is di-substituted or tri-substituted phenyl, then each R⁸ is independently (C₁-C₆) alkyl, (C₁-C₆) alkoxy, methoxy, halo, chloro, —(C₁-C₆) perhaloalkyl, —CF₃, (C₁-C₆) perhaloalkoxy, or —OCF₃. Preferably, each R⁸ is independently methoxy, chloro, —CF₃, or —OCF₃

In a preferred embodiment, R² is di-substituted phenyl and the R⁸ substituents are positioned 3, 4 or 3,5. Specific examples of preferred di-substituted phenyl rings include 3-chloro-4-methoxy-phenyl, 3-methoxy-4-chlorophenyl, 3-chloro-4-trifluoromethoxy-phenyl, 3-trifluoromethoxy-4-chloro-phenyl, 3,4-dichloro-phenyl, 3,4-dimethoxyphenyl and 3,5-dimethoxyphenyl.

In another specific embodiment of the preceding embodiment are provided compounds of formulas (III) and (IIa) with the provisos that:

(1) when R⁴ is one of the above-identified phenyls, and R⁵ and R⁶ are each hydrogen, then R² is not 3,4,5-tri(C₁-C₆)alkoxyphenyl or 3,4,5-trimethoxyphenyl;

(2) when R² is 3,4-dimethoxyphenyl and R⁵ and R⁶ are each hydrogen, then R⁴ is not 3-(C₁-C₆)alkoxyphenyl, 3-methoxyphenyl, 3,4-di-(C₁-C₆) alkoxyphenyl or 3,4-dimethoxyphenyl;

(3) when R⁴ is 3-chloro-4-methoxyphenyl and R⁵ is halo or fluoro, and optionally R⁶ is hydrogen, then R² is not 3-chloro-4-(C₁-C₆) alkoxyphenyl or 3-chloro-4-methoxyphenyl;

(4) when R⁴ is 3,4-dichlorophenyl, R⁵ is hydrogen, (C₁-C₆) alkyl, methyl, halo or chloro and optionally R⁶ is hydrogen, then R² is not a phenyl mono substituted at the para position with a (C₁-C₆) alkoxy group which is optionally substituted with one or more of the same or different R^{b}, —OH or —NR^{c}R^{c} groups, where R^{b} and R^{c} are as previously described for structural formula (I); and/or

(5) R² and/or R⁴ is not 3,4,5-tri(C₁-C₆) alkoxyphenyl or 3,4,5-trimethoxyphenyl, especially when R⁵ and R⁶ are each hydrogen.

In another embodiment, when R² is tri-substituted phenyl, then the trisubstituted phenyl has the formula (XL):

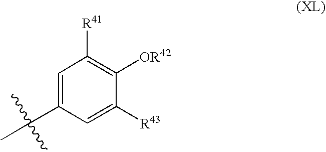

wherein: R⁴¹ is methyl or (C₁-C₆) alkyl; R⁴² is hydrogen, methyl, or (C₁-C₆) alkyl; and R⁴³ is a halo group.

In a thirteenth embodiment of the compounds of structural formulae (II), (IIa), (III), and (IIIa), R² and/or R⁴ is an optionally substituted heteroaryl. Preferably, heteroaryl groups according to this thirteenth embodiment comprise from 5 to 15, and more preferably from 5 to 11 ring atoms, and include one, two, three or four of the same or different heteratoms or heteroatomic groups that are each independently N, NH, O, S, S(O), or S(O)₂. The optionally substituted heteroaryl may be attached to its respective C2 or C4 nitrogen atom or linker L¹ or L² through any available carbon atom or heteroatom, but is typically attached via a carbon atom. The optional substituents can be the same or different, and can be attached to any available carbon atom or heteroatom.

In one embodiment of compounds of the thirteenth embodiment, R⁵ is other than bromo, nitro, trifluoromethyl, cyano or —C(O)NHR, where R is hydrogen or (C₁-C₆) alkyl.

In another embodiment of compounds of the thirteenth embodiment, when R² and R⁴ are each a substituted or unsubstituted pyrrole or indole, then the ring is attached to the remainder of the molecule via a ring carbon atom.

In still another embodiment of compounds of the thirteenth embodiment, the heteroaryl is unsubstituted or substituted with from one to four of the same or different R⁸ groups, where each R⁸ is as previously defined for structural formula (II) and (III).

In one embodiment of compounds of the thirteenth embodiment, R² and/or R⁴ is an optionally substituted 5-15 membered heteroaryl wherein each R⁸ is independently R^{d}, —NR^{c}R^{c}, —(CH₂)ₘ—NR^{c}R^{c}, —C(O)NR^{c}R^{c}, —(CH₂)ₘ—C(O)NR^{c}R^{c}, —C(O)OR^{d}, —(CH₂)ₘ—C(O)OR^{d}, or —(CH₂)ₘ—OR^{d}, where m, R^{c} and R^{d} are as previously defined for structural formula (II) and (III).

Specific examples of optionally substituted heteroaryls of the thirteenth embodiment include, but are not limited to, the following heteroaryl groups of formulae (XLI)-(LXXXVIII):

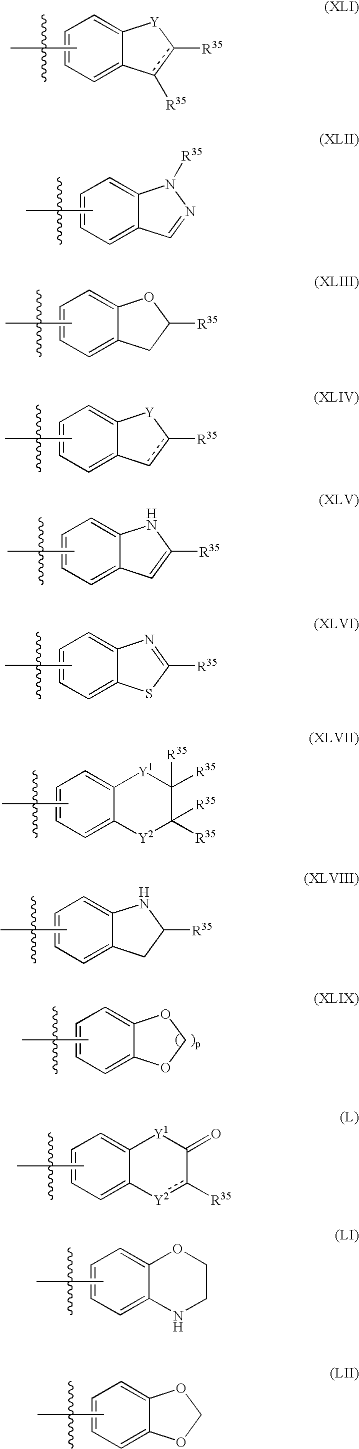

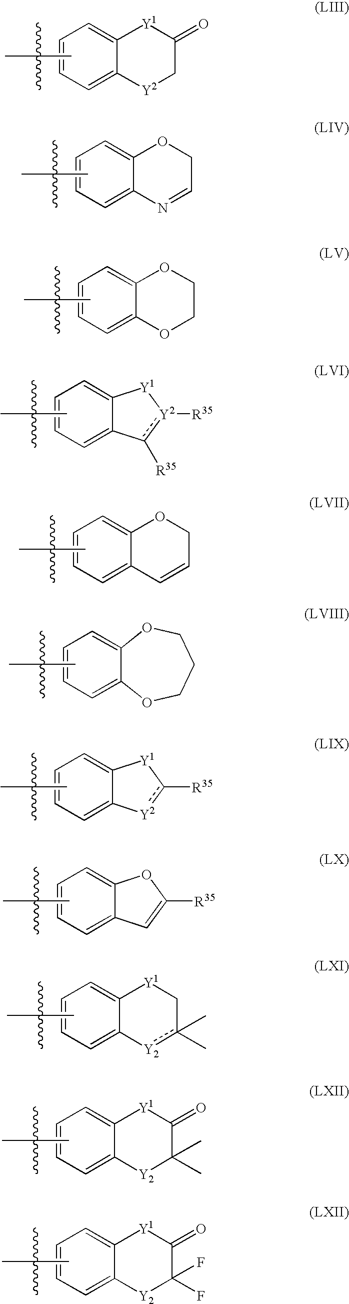

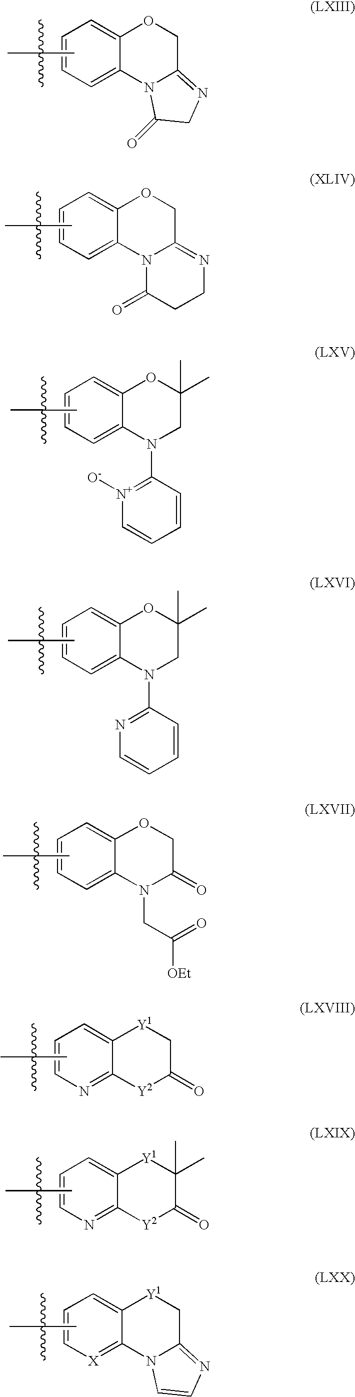

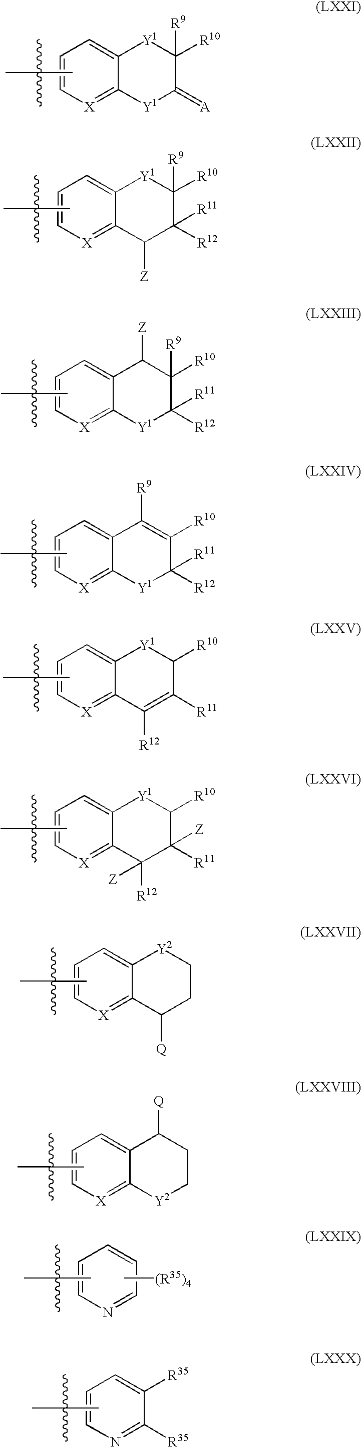

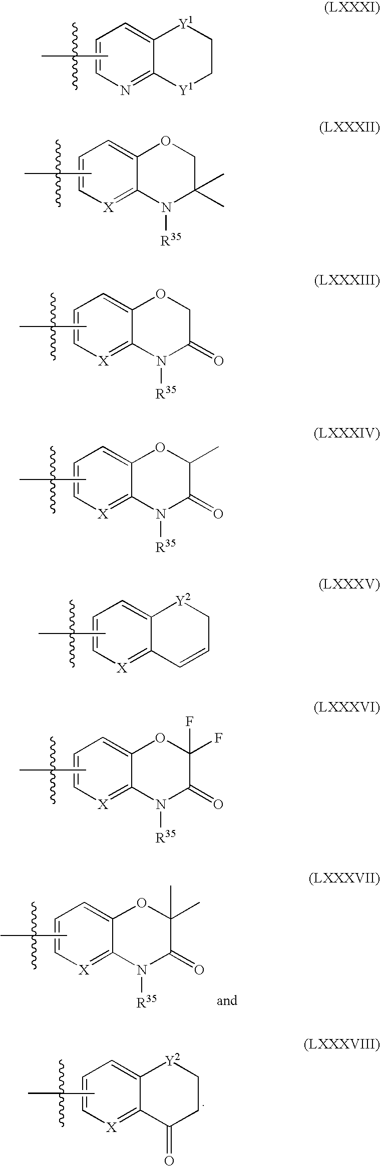

wherein:
p is an integer from one to three;
each . . . independently represents a single bond or a double bond;
R³⁵ is hydrogen or R⁸, where R⁸ is as previously defined for structural formula (II);
X is CH, N, or N—O;
each Y is independently O, S, or NH;
each Y¹ is independently O, S, SO, SO₂, SONR³⁶, NH, or NR¹⁷;
each Y² is independently CH, CH₂, O, S, N, NH, or NR¹⁷;
R³⁶ is hydrogen or alkyl;
R³⁷ is hydrogen or a progroup, preferably hydrogen or a progroup that is aryl, arylalkyl, heteroaryl, R^{a}, R^{b}—CR^{a}R^{b} —O—C(O)R⁸, —CR^{a}R^{b}—O—PO(OR⁸)₂, —CH₂—O—PO(OR²)₂, —CH₂—PO(OR⁸)₂, —C(O)—CR^{a}R^{b}—N(CH₃)₂, —CR^{a}R^{b}—O—C(O)— —CR^{a}R^{b}—N(CH₃)₂, —C(O)R⁸, —C(O)CF₃, or —C(O)—NR⁸—C(O)R⁸;
A is O, NH, or NR³⁸;
R³⁸ is alkyl or aryl;
R⁹, R¹⁰, R¹¹ and R¹² are each independently alkyl, alkoxy, halogen, haloalkoxy, aminoalkyl or hydroxyalkyl;
or R⁹ and R¹⁰ and/or R¹¹ and R¹² taken together form a ketal;
each Z is independently hydroxyl, alkoxy, aryloxy, ester, carbamate or sulfonyl;
Q is —OH, OR⁸, —NR^{c}R^{c}, —NHR¹⁹—C(O)R⁸, —NHR³⁹—C(O)OR⁸, —NR¹⁹—CHR⁴⁰—R^{b}, —NR³⁹—(CH₂)ₘ—R^{b}, or —NR³⁹—C(O)—CHR⁴⁰—NR^{c}R^{c};
R³⁹ and R⁴⁰ are each independently hydrogen, alkyl, aryl, alkylaryl; arylalkyl, or NHR⁸; and
R^{a}, R^{b} and R^{c} are as previously defined for structural formula (II).

In one embodiment of formulae (LXXVII) and (LXXVIII), R^{b} substitutents for Q are each independently —C(O)OR⁸, —O—C(O)R⁸, —O—P(O)(OR⁸)₂, or —P(O)(OR⁸)₂.

In another embodiment of formulae (XLI)-(LXXXVIII), each R⁸ is independently R^{d}, —NR^{c}R^{c}, —(CH₂)ₘ—NR^{c}R^{c}, —C(O)NR^{c}R^{c}, —(CH₂)ₘ—C(O)NR^{c}R^{c}, —C(O)OR^{d}, —(CH₂)ₘ—C(O)OR^{d}, or —(CH₂)ₘ—OR^{d}, where m, R^{c} and R^{d} are as previously defined for structural formula (II) and (III).

In another embodiment of formulae (XLI)-(LXXXVIII), R^{d} and/or R^{c} is (i) R^{a} or (ii) (C₃-C₈) cycloalkyl optionally substituted with one or more of the same or different hydroxyl, amino or carboxyl groups.

In another embodiment of formulae (XLI)-(LXXXVIII), each R³⁵ is hydrogen or (C₁-C₆) ethyl or methyl.

In another embodiment of formulae (XLI)-(LXXXVIII), the aromatic ring connectivity is either at the 5 or 6 position. It should be understood that either R² or R⁴ can utilize the heteroaryl groups discussed throughout this specification.

In a another embodiment of the compounds of structural formulae (II), (IIa), (III), and (IIIa), R² and R⁴ are each independently an optionally substituted phenyl, aryl or heteroaryl, with the provisos for formulas (III) and (IIa) that:
(1) when L¹ is a direct bond and R⁶ and optionally R⁵ is hydrogen, then R² is other than 3,4,5-trimethoxyphenyl or 3,4,5-tri(C₁-C₆) alkoxyphenyl;
(2) when L¹ and L² are each a direct bond, R⁶ is hydrogen and R⁵ is halo, then R² and R⁴ are not each simultaneously 3,4,5-trimethoxyphenyl or 3,4,5-tri(C₁-C₆) alkoxyphenyl;
(3) when R⁴ is 3-methoxyphenyl or 3-(C₁-C₆) alkoxyphenyl and R² is a 3,4,5-trisubstituted phenyl, the substituents positioned at the 3 and 4 positions are not both simultaneously methoxy or (C₁-C₆) alkoxy;
(4) when R² is a substituted phenyl and R⁶ is hydrogen, then R⁵ is other than cyano or C(O)NHR, where R is hydrogen or (C₁-C₆) alkyl; and/or
(5) when R² and R⁴ are each independently a substituted or unsubstituted pyrrole or indole, then the pyrrole or indole is attached to the remainder of the molecule via a ring carbon atom.

Alternatively, R² is subject to the provisos described in connection with the first or second embodiment.

In this embodiment of the invention, the R² and R⁴ substituents can be the same or different. Specific optionally substituted phenyl, aryl and/or heteroaryls include those illustrated above in connection with the twelfth and thirteenth embodiments.

In another embodiment of the compounds of structural formulae (II), (IIa), (III), and (IIIa), including the above-described first through fourteenth embodiments thereof, R⁶ is hydrogen and R⁵ is an electronegative group. As will be recognized by skilled artisans, electronegative groups are atoms or groups of atoms that have a relatively great tendency to attract electrons to themselves. Specific examples of electronegative groups according to this fourteenth embodiment include, but are not limited to, —CN, —NC, —NO₂, halo, bromo, chloro, fluoro, (C₁-C₃) haloalkyl, (C₁-C₃) perhaloalkyl, (C₁-C₃) fluoroalkyl, (C₁-C₃) perfluoroalkyl, —CF₃, (C₁-C₃) haloalkoxy, (C₁-C₃) perhaloalkoxy, (C₁-C₃) fluoroalkoxy, (C₁-C₃) perfluoroalkoxy, —OCF₃, —C(O)R³, —C(O)OR^{a}, —C(O)CF₃ and —C(O)OCF₃. In a specific embodiment, the electronegative group is a halogen-containing electronegative group, such as —OCF₃, —CF₃, bromo, chloro or fluoro. In another specific embodiment, R⁵ is fluoro, subject to the proviso that the compound is not any compound according to the third embodiment.

In a another embodiment, the compounds of structural formulae (II), (IIa), (III), and (IIIb) are compounds according to structural formula (IIb) and (IIIb):

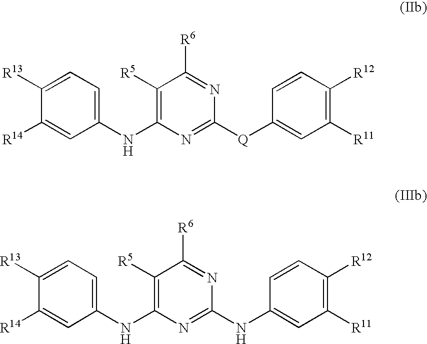

and pharmaceutically acceptable salts, hydrates, solvates and N-oxides thereof wherein

R¹¹, R¹², R¹³ and R¹⁴ are each independently hydrogen, hydroxy, (C₁-C₆) alkoxy or —NR^{c}R^{c}; and

R⁵, R⁶ and R^{c} are as previously defined for structural formula (II) and (III), with the proviso that when R¹³, R⁵ and R⁶ are each hydrogen, then R¹¹ and R¹² are not simultaneously methoxy, (C₁-C₆) alkoxy or (C₁-C₆) haloalkoxy.

In a another embodiment, the compounds of structural formulae (II), (IIa), (III), and (IIIb), are compounds according to structural formula (IIc) and (IIIc):

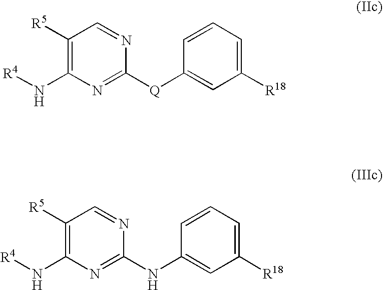

and pharmaceutically acceptable salts, hydrates, solvates and N-oxides thereof, wherein:
R⁴ is a 5-10 membered heteroaryl or 3-hydroxyphenyl;
R⁵ is F or —CF₃; and
R⁸ is —O(CH₂)ₘ—R^{b}, where m and R^{b} are as previously defined for structural formula (II) and (III).

In a specific embodiment, R⁵ is —O—CH₂—C(O)NH—CH₃ and/or R⁴ is a heteroaryl according to the thirteenth embodiment.

In all of the compounds described herein that include substituent alternatives that may be substituted, such as, for example, some of the substituent alternatives delineated for R^{d}, R^{e}, R^{f}, R^{g}, and R^{h}, the substitutions are typically, independently of one another, selected from amongst the R^{b} groups described in connection with structural formulae (I), (II) and (III). In a specific embodiment, any present substitutions are, independently of one another, selected from hydroxyl, lower alkoxy, (C₆-C₁₄) aryloxy, lower alkoxyalkyl, methoxymethyl, methoxyethyl, ethoxymethyl, ethoxyethyl and halogen.

Moreover, skilled artisans will appreciate that when lists of alternative substituents include members which, owing to valency requirements or other reasons, cannot be used to substitute a particular group, the list is intended to be read in context to include those members of the list that are suitable for substituting the particular group. For example, skilled artisans will appreciate that while all of the listed alternatives for R^{b} can be used to substitute an alkyl group, certain of the alternatives, such as ═O, cannot be used to substitute a phenyl group. It is to be understood that only possible combinations of substituent-group pairs are intended.

Depending upon the nature of the various substituents, the compounds and/or prodrugs described herein may be in the form of salts. Such salts include salts suitable for pharmaceutical uses (“pharmaceutically-acceptable salts”), salts suitable for veterinary uses, etc. Such salts may be derived from acids or bases, as is well-known in the art.

In one embodiment, the salt is a pharmaceutically acceptable salt. Generally, pharmaceutically acceptable salts are those salts that retain substantially one or more of the desired pharmacological activities of the parent compound and which are suitable for administration to humans. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, hydriodic, etc.), sulfuric acid, nitric acid, phosphoric acid, and the like. Organic acids suitable for forming pharmaceutically acceptable acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl)benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-toluenesulfonic acid, camphorsulfonic acid, etc.), 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

Pharmaceutically acceptable salts also include salts formed when an acidic proton present in the parent compound is either replaced by a metal ion (e.g., an alkali metal ion, an alkaline earth metal ion or an aluminum ion) or coordinates with an organic base (e.g., ethanolamine, diethanolamine, triethanolamine, N-methylglucamine, morpholine, piperidine, dimethylamine, diethylamine, etc.).

The compounds and/or prodrugs described herein, as well as the salts thereof, may also be in the form of hydrates, solvates and N-oxides, as are well-known in the art. Unless specifically indicated otherwise, the expression “prodrug” is intended to encompass such salts, hydrates, solvates and/or N-oxides. Specific exemplary salts include, but are not limited to, mono- and di-sodium salts, mono- and di-potassium salts, mono- and di-lithium salts, mono- and di-alkylamino salts, mono-magnesium salts, mono-calcium salts and ammonium salts.

Compounds can be tested in various biochemical and cellular assays for their inhibitory effect on autophosphorylation of RET kinase. In other embodiments, the activity is determined by contacting an isolated RET kinase, or an active fragment thereof with an inhibitor compound in the presence of a RET kinase substrate (e.g., ATP) and assessing RET phosphorylation. Alternatively, the assay can be carried out with cells that express a RET kinase. The cells can express the RET kinase endogenously or they can be engineered to express a recombinant RET kinase. The cells can optionally also express the RET kinase substrate. In other embodiments the compounds can be tested for their effect on RET kinase substrate phosphorylation or downstream activation, e.g. phosphorylation of AKT or activation of PLK3. Cells suitable for performing such confirmation assays, as well as methods of engineering suitable cells will be apparent to those of skill in the art.

Determining the effect of the inhibitor compounds on cell proliferation can use any number of in vitro and in vivo assays. For example, proliferating cells can be suitably cultured in vitro and treated with the compounds of interest. Proliferative capacity in the cell populations can be determined use dye staining (e.g., trypan blue dye-exclusion; 3-4,5-dimethylthiazol-2,5-diphenyltetrazolium (MTT); and annexin V), or cell sorting techniques (e.g., fluorescence activated cell sorting with propidium iodide). In vivo assays for cell proliferation can be based on transplantation of tumor cells into experimental animals followed by administration of the inhibitor compounds. These and other methods of assessing cell proliferation will be apparent to the skilled artisan.

In Vitro Uses

To assess the antiproliferative effects of compounds of formula (XI) and prodrug compounds of formulae (II), (III), (XII), and (XIII), on growth of particular cancer cell lines, the compounds can be administered by contacting cultured tumor cell lines with the compounds. In the context of in vitro assays, administration of the drug or prodrug compound to tumor cells may be simply contacting cells in culture with an amount of the drug or prodrug compound in an amount effective to inhibit proliferation. When the drug compound is supplied in the form of a prodrug compound, the method is carried out under conditions in which the prodrug compound yields the drug compound.

Examples of tumor cell lines derived from human tumors and available for use in the in vivo studies described herein include, but are not limited to, leukemia cell lines (e.g., CCRF-CEM, HL-60(TB), K-562, MOLT-4, RPM1-8226, SR, P388 and P388/ADR); non-Small cell lung cancer cell lines (e.g., A549/ATCC, EKVX, HOP-62, HOP-92, NCI-H226, NCI-H23, NCI-H322M, NCI-H460, NCI-H522 and LXFL 529); small cell lung cancer cell lines (e.g., DMS 114 and SHP-77); colon cancer cell lines (e.g., COLO 205, HCC-2998, HCT-116, HCT-15, HT29, KM12, SW-620, DLD-1 and KM20L2); Central Nervous System (CNS) cancer cell lines (e.g., SF-268, SF-295, SF-539, SNB-19, SNB-75, U251, SNB-78 and XF 498); melanoma cell lines (e.g., LOX I MVI, MALME-3M, M14, SK-MEL-2, SK-MEL-28, SK-MEL-5, UACC-257, UACC-62, RPMI-7951 and M19-MEL); ovarian cancer cell lines (e.g., IGROV1, OVCAR-3, OVCAR-4, OVCAR-5, OVCAR-8 and SK-OV-3); renal cancer cell lines (e.g., 786-0, A498, ACHN, CAKI-1, RXF 393, SN12C, TK-10, UO-31, RXF-631 and SN12K1); prostate cancer cell lines (e.g., PC-3 and DU-145); breast cancer cell lines (e.g., MCF7, NCI/ADR-RES, MDA-MB-231/ATCC, HS 578T, MDA-MB-435, BT-549, T-47D and MDA-MB-468); and thyroid cancer cell lines (e.g., SK-N-SH).

In Vivo Uses

Compounds of formula (XI) and prodrug compounds of formulae (II), (III), (XII), and (XIII) can be used to inhibit tumor cell growth in a subject, as a therapeutic approach towards the treatment or prevention of proliferative disorders, such as tumorigenic cancers.

Generally, cell proliferative disorders treatable with the compounds provided herein relate to any disorder characterized by aberrant cell proliferation. These include various tumors and cancers, benign or malignant, metastatic or non-metastatic.

Types of Cancers

A variety of cellular proliferative disorders may be treated using the drug and prodrug compounds via the disclosed methods. In some embodiments, the drug or prodrug compounds are used to treat various cancers in afflicted subjects. Cancers are traditionally classified based on the tissue and cell type from which the cancer cells originate. Carcinomas are considered cancers arising from epithelial cells while sarcomas are considered cancers arising from connective tissues or muscle. Other cancer types include leukemias, which arise from hematopoietic cells, and cancers of nervous system cells, which arise from neural tissue. For non-invasive tumors, adenomas are considered benign epithelial tumors with glandular organization while chondomas are benign tumor arising from cartilage. In the present invention, the described compounds may be used to treat proliferative disorders encompassed by carcinomas, sarcomas, leukemias, neural cell tumors, and non-invasive tumors.

Solid tumor cancers include malignant neoplastic masses of tissue or cancerous neoplasms characterized by the progressive or uncontrolled proliferation of cells. The cells involved in the neoplastic growth have an intrinsic heritable abnormality such that they are not regulated properly by normal methods. Malignant or cancerous neoplasms tend to grow rapidly, spread throughout the body, and recur if removed. The cells of malignant tumors may be well differentiated, but most have some degree of anaplasia. Anaplastic cells tend to be larger than normal and are abnormal, even bizarre, in shape. The nuclei tend to be very large, and irregular, and they often stain darkly. Malignant tumors may be partially encapsulated, but the cells of the cancer can infiltrate and destroy surrounding tissue. Thus, cells from the primary tumor can migrate (metastasize) from the original tumor site and colonize in other tissues. Tumors formed from cells that have spread are referred to as “secondary tumors” and contain cells that are similar to those in the original “primary” tumor. Metastatic tumors typically form by migration of tumor cells from the original tumor site through the blood and lymph system to other tissues.

Specific properties of cancers, such as tissue invasiveness or metastasis, may be targeted using the methods described herein. In some embodiments, the drugs or prodrugs are used to treat solid tumors arising from various tissue types, including, but not limited to, cancers of the bone, breast, respiratory tract (e.g., bladder), brain reproductive organs, digestive tract, urinary tract, eye, liver, skin, head, neck, thyroid, parathyroid, and metastatic forms thereof.

Specific proliferative disorders include the following:

a) proliferative disorders of the breast include, but are not limited to, invasive ductal carcinoma, invasive lobular carcinoma, ductal carcinoma, lobular carcinoma in situ, and metastatic breast cancer;

b) proliferative disorders of the skin include, but are not limited to, basal cell carcinoma, squamous cell carcinoma, malignant melanoma, and Karposi's sarcoma;

c) proliferative disorders of the respiratory tract include, but are not limited to, small cell and non-small cell lung carcinoma, bronchial adema, pleuropulmonary blastoma, and malignant mesothelioma;

d) proliferative disorders of the brain include, but are not limited to, brain stem and hyptothalamic glioma, cerebellar and cerebral astrocytoma, medullablastoma, ependymal tumors, oligodendroglial, meningiomas, and neuroectodermal and pineal tumors;

e) proliferative disorders of the male reproductive organs include, but are not limited to, prostate cancer, testicular cancer, and penile cancer;

f) proliferative disorders of the female reproductive organs include, but are not limited to, uterine cancer (endometrial), cervical, ovarian, vaginal, vulval cancers, uterine sarcoma, ovarian germ cell tumor;

g) proliferative disorders of the digestive tract include, but are not limited to, anal, colon, colorectal, esophageal, gallbladder, stomach (gastric), pancreatic cancer, pancreatic cancer-Islet cell, rectal, small-intestine, and salivary gland cancers;

h) proliferative disorders of the liver include, but are not limited to, hepatocellular carcinoma, cholangiocarcinoma, mixed hepatocellular cholangiocarcinoma, and primary liver cancer;

i) proliferative disorders of the eye include, but are not limited to, intraocular melanoma, retinoblastoma, and rhabdomyosarcoma;

j) proliferative disorders of the head and cancers include, but are not limited to, laryngeal, hypopharyngeal, nasopharyngeal, oropharyngeal cancers, and lip and oral cancer, squamous neck cancer, metastatic paranasal sinus cancer;

k) proliferative disorders of the lymphomas include, but are not limited to, various T cell and B cell lymphomas, non-Hodgkins lymphoma, cutaneous T cell lymphoma, Hodgkins disease, and lymphoma of the central nervous system;

l) leukemias include, but are not limited to, acute myeloid leukemia, acute lymphoblastic leukemia, chronic lymphocytic leukemia, chronic myelogenous leukemia, and hair cell leukemia,

m) proliferative disorders of the thyroid include thyroid cancer, thymoma, malignant thymoma, medullary thyroid carcinomas, papillary thyroid carcinomas, multiple endocrine neoplasia type 2A (MEN2A), pheochromocytoma, parathyroid adenomas, multiple endocrine neoplasia type 2B (MEN2B), familial medullary thyroid carcinoma (FMTC) and carcinoids;

n) proliferative disorders of the urinary tract include, but are not limited to, bladder cancer;

o) sarcomas include, but are not limited to, sarcoma of the soft tissue, osteosarcoma, malignant fibrous histiocytoma, lymphosarcoma, and rhabdomyosarcoma;

p) proliferative disorders of the kidneys include, but are not limited to, renal cell carcinoma, clear cell carcinoma of the kidney; and renal cell adenocarcinoma;

q) precursor B-lymphoblastic leukemia/lymphoma (precursor B-cell acute lymphoblastic leukemia), are B-cell chronic lymphocytic leukemia/small lymphocytic lymphoma, B-cell prolymphocytic leukemia, lymphoplasmacytic lymphoma, splenic marginal zone B-cell lymphoma, hairy cell leukemia, plasma cell myeloma/plasmacytoma, extranodal marginal zone B-cell lymphoma of MALT type, nodal marginal zone B-cell lymphoma, follicular lymphoma, mantle-cell lymphoma, diffuse large B-cell lymphoma, mediastinal large B-cell lymphoma, primary effusion lymphoma, and Burkitt's lymphoma/Burkitt cell leukemia

(r) precursor T-lymphoblastic lymphoma/leukemia (precursor T-cell acute lymphoblastic leukemia) T-cell prolymphocytic leukemia T-cell granular lymphocytic leukemia, aggressive NK-cell leukemia, adult T-cell lymphoma/leukemia (HTLV-1), extranodal NK/T-cell lymphoma, nasal type, enteropathy-type T-cell lymphoma, hepatosplenic gamma-delta T-cell lymphoma, subcutaneous panniculitis-like T-cell lymphoma, Mycosis fungoides/Sezary syndrome, Anaplastic large-cell lymphoma, T/null cell, primary cutaneous type, Peripheral T-cell lymphoma, not otherwise characterized, Angioimmunoblastic T-cell lymphoma, Anaplastic large-cell lymphoma, T/null cell, primary systemic type;

(s) nodular lymphocyte-predominant Hodgkin's lymphoma, Nodular sclerosis Hodgkin's lymphoma (grades 1 and 2), Lymphocyte-rich classical Hodgkin's lymphoma, Mixed cellularity Hodgkin's lymphoma, and Lymphocyte depletion Hodgkin's lymphoma;

(t) myelogenous leukemia (e.g., Philadelphia chromosome positive (t(9; 22)(qq34; q11)), multiple myeloma, chronic neutrophilic leukemia, chronic eosinophilic leukemia/hypereosinophilic syndrome, chronic idiopathic myelofibrosis, polycythemia vera, and essential thrombocythemia, chronic myelomonocytic leukemia, atypical chronic myelogenous leukemia, and juvenile myelomonocytic leukemia, are refractory anemia, with ringed sideroblasts and without ringed sideroblasts, refractory cytopenia (myelodysplastic syndrome) with multilineage dysplasia, refractory anemia (myelodysplastic syndrome) with excess blasts, 5q-syndrome, and myelodysplastic syndrome with t(9; 12)(q22; p12);

(u) AML with t(8; 21)(q22; q22), AML1(CBF-alpha)/ETO, Acute promyelocytic leukemia (AML with t(15; 17)(q22; q11-12) and variants, PML/RAR-alpha), AML with abnormal bone marrow eosinophils (inv(16)(p13q22) or t(16; 16)(p13; q11), CBFb/MYH11X), and AML with 11q23 (MLL) abnormalities, AML minimally differentiated, AML without maturation, AML with maturation, Acute myelomonocytic leukemia, Acute monocytic leukemia, Acute erythroid leukemia, Acute megakaryocytic leukemia, Acute basophilic leukemia, and Acute panmyelosis with myelofibrosis

It is to be understood that the descriptions of proliferative disorders is not limited to the conditions described above, but encompasses other disorders characterized by uncontrolled growth and malignancy. It is further understood that proliferative disorders include various metastatic forms of the tumor and cancer types described herein. The drug and prodrug compounds of the described methods may be tested for effectiveness against these disorders, and a therapeutically effective regimen established. Effectiveness, as further described below, includes reduction or remission of the tumor, decreases in the rate of cell proliferation, or cytostatic or cytotoxic effect on cell growth.

Preferred proliferative disorders include proliferative disorders of the thyroid include thyroid cancer, thymoma, malignant thymoma, medullary thyroid carcinomas, papillary thyroid carcinomas, multiple endocrine neoplasia type 2A (MEN2A), pheochromocytoma, parathyroid adenomas, multiple endocrine neoplasia type 2B (MEN2B), and familial medullary thyroid carcinoma (FMTC).

Other preferred proliferative disorders include proliferative disorders of the kidneys include, but are not limited to, renal cell carcinoma, clear cell carcinoma of the kidney; and renal cell adenocarcinoma.

Methods of Synthesis

Compounds for use in the methods of the invention may be readily prepared by one skilled in the art as discussed in detail in U.S. application Ser. No. 11/453,731, filed Jun. 14, 2006 (US2006/0234983A1), in U.S. application Ser. No. 11/337,049, filed Jan. 19, 2006 (US 2006/0211657A1), and U.S. application Ser. No. 10/355,543, filed Jan. 31, 2003 (US2004/0029902A1), which are hereby incorporated by reference in their entirety.

In particular, the metabolism of a 2,4-pyrimidinediamine prodrug (Compound P-1 to Compound 1, supra) of the instant disclosure is detailed in U.S. application Ser. No. 11/337,049 filed Jan. 19, 2006 (US2006/0211657 A1), at paragraphs 134-142 and 146 of the printed publication.

Dosages

The active compound(s), prodrugs, or compositions thereof, can be used in an amount effective to treat or prevent the particular disease being treated. The compound(s), prodrugs, or compositions thereof can be administered therapeutically to achieve therapeutic benefit or prophylactically to achieve prophylactic benefit. By therapeutic benefit is meant eradication or amelioration of the underlying cell proliferative disorder being treated (e.g., medullary thyroid carcinomas, papillary thyroid carcinomas, multiple endocrine neoplasia type 2A (MEN2A), parathyroid adenomas, multiple endocrine neoplasia type 2B (MEN2B), familial medullary thyroid carcinoma (FMTC), pheochromocytoma and parathyroid hyperplasia.) and/or eradication or amelioration of one or more of the symptoms associated with the underlying disorder such that the patient reports an improvement in condition, notwithstanding that the patient can still be afflicted with the underlying disorder. Therapeutic benefit also includes halting or slowing the progression of the disease, regardless of whether improvement is realized.

For prophylactic administration, the active compound, prodrug, or composition thereof, can be administered to a patient at risk of developing a disorder characterized by, caused by or associated with aberrant cell proliferation, such as the various disorders previously described above. For instance, if a patient is diagnosed with a tumor but there is no indication of metastasis, the inhibitor compounds can be administered prophylactically to inhibit tumor metastasis.

The amount of the active compound, prodrug, or composition thereof, administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, etc. Determination of an effective dosage is well within the capabilities of those skilled in the art.

Initial dosages can be estimated initially from in vitro assays. For example, an initial dosage for use in animals can be formulated to achieve a circulating blood or serum concentration of compound that inhibits RET sufficient to reduce the cell proliferation or invasiveness of the tumor cells. Alternatively, an initial dosage for use in animals can be formulated to achieve a circulating blood or serum concentration of active compound that is equal to or greater than the IC₅₀ as measured in RET kinase inhibition assay. Calculating dosages to achieve such circulating blood or serum concentrations taking into account the bioavailability of the particular inhibitor compound is well within the capabilities of skilled artisans. For guidance, the reader is referred to Fingl and Woodbury, “General Principles,” In: The Pharmaceutical Basis of Therapeutics, Chapter 1, pp. 1-46, 1975, and the references cited therein. Initial dosages can also be estimated from in vivo data, such as animal models. Animal models useful for testing the efficacy of compounds to treat or prevent diseases characterized by, caused by or associated with RET kinase activity are described herein.

Dosage amounts will typically be in the range of from about 1 mg/kg/day to about 100 mg/kg/day, 200 mg/kg/day, 300 mg/kg/day, 400 mg/kg/day or 500 mg/kg/day, but can be higher or lower, depending upon, among other factors, the activity of the inhibitory compound, its bioavailability, the mode of administration and various factors discussed above. Dosage amount and interval can be adjusted individually to provide plasma levels of the active compound(s) which are sufficient to maintain therapeutic or prophylactic effect. In cases of local administration or selective uptake, such as local topical administration, the effective local concentration of active compound(s) may not be related to plasma concentration. Skilled artisans will be able to optimize effective dosages without undue experimentation.

The active compounds, prodrugs, or compositions thereof, can be administered once per day, a few or several times per day, or even multiple times per day, depending upon, among other things, the indication being treated and the judgment of the prescribing physician.

Preferably, the active the active compounds, prodrugs, or compositions thereof, will provide therapeutic or prophylactic benefit without causing substantial toxicity. Toxicity of the active compound(s) can be determined using standard pharmaceutical procedures. The dose ratio between toxic and therapeutic (or prophylactic) effect is the therapeutic index. Active compound(s) that exhibit high therapeutic indices are preferred.

Administration

When used to treat or prevent cell proliferative disorders, the RET kinase inhibitor compounds and prodrugs can be administered singly, as mixtures of one or more active compounds and/or prodrugs or in mixture or combination with other agents useful for treating such diseases and/or symptoms associated with such diseases. The compounds and prodrugs can be administered per se or as pharmaceutical compositions.

Pharmaceutical compositions comprising the compounds and/or prodrugs of the invention can be manufactured by means of conventional mixing, dissolving, granulating, dragee-making levigating, emulsifying, encapsulating, entrapping or lyophilization processes. The compositions can be formulated in conventional manner using one or more physiologically acceptable carriers, diluents, excipients or auxiliaries which facilitate processing of the active compounds into preparations which can be used pharmaceutically. The actual pharmaceutical composition administered will depend upon the mode of administration. Virtually any mode of administration can be used, including, for example topical, oral, systemic, inhalation, injection, transdermal, etc.

The active compound and/or prodrug can be formulated in the pharmaceutical compositions per se, or in the form of a pharmaceutically acceptable salt. As used herein, the expression “pharmaceutically acceptable salt” means those salts which retain substantially the biological effectiveness and properties of the active compound and which is not biologically or otherwise undesirable. Such salts can be prepared from inorganic and organic acids and bases, as is well-known in the art. Typically, such salts are more soluble in aqueous solutions than the corresponding free acids and bases.

For topical administration, the active compound(s) and/or prodrug(s) can be formulated as solutions, gels, ointments, creams, suspensions, etc. as are well-known in the art.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) and/or prodrug(s) in aqueous or oily vehicles. The compositions can also contain formulating agents, such as suspending, stabilizing and/or dispersing agent. The formulations for injection can be presented in unit dosage form, e.g., in ampules or in multidose containers, and can contain added preservatives.

Alternatively, the injectable formulation can be provided in powder form for reconstitution with a suitable vehicle, including but not limited to sterile pyrogen free water, buffer, dextrose solution, etc., before use. To this end, the active compound(s) and/or prodrug(s) can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art with, for example, sugars or enteric coatings.

Liquid preparations for oral administration can take the form of, for example, elixirs, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound(s) and/or prodrug(s).

For buccal administration, the compositions can take the form of tablets or lozenges formulated in conventional manner.

For rectal and vaginal routes of administration, the active compound(s) and/or prodrug(s) can be formulated as solutions (for retention enemas) suppositories or ointments containing conventional suppository bases such as cocoa butter or other glycerides.

For administration by inhalation, the active compound(s) and/or prodrug(s) can be conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated containing a powder mix of the active compound(s) and/or prodrug(s) and a suitable powder base such as lactose or starch.

For prolonged delivery, the active compound(s) and/or prodrug(s) can be formulated as a depot preparation, for administration by implantation; e.g., subcutaneous, intradermal, or intramuscular injection. Thus, for example, the active compound(s) and/or prodrug(s) can be formulated with suitable polymeric or hydrophobic materials (e.g., as an emulsion in an acceptable oil) or ion exchange resins, or as sparingly soluble derivatives; e.g., as a sparingly soluble salt.

Alternatively, transdermal delivery systems manufactured as an adhesive disc or patch which slowly releases the active compound(s) for percutaneous absorption can be used. To this end, permeation enhancers can be used to facilitate transdermal penetration of the active compound(s). Suitable transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other pharmaceutical delivery systems can be employed. Liposomes and emulsions are well-known examples of delivery vehicles that can be used to deliver active compounds(s) and/or prodrug(s). Certain organic solvents such as dimethylsulfoxide (DMSO) can also be employed, although usually at the cost of greater toxicity.

The pharmaceutical compositions can, if desired, be presented in a pack or dispenser device which can contain one or more unit dosage forms containing the active compound(s). The pack can, for example, comprise metal or plastic foil, such as a blister pack. The pack or dispenser device can be accompanied by instructions for administration.

Formulations

When used to treat or prevent a solid tumor cancer, the active compounds(s) and/or prodrug(s) of formulae (I), (II), (III), (XI), (XII), and (XIII) can be administered singly, as mixtures of one or more active compounds and/or prodrugs or in mixture or combination with other agents useful for treating cancer and/or the symptoms associated with cancer. Compounds of formulae (I), (II), (III), (XI), (XII), and/or (XIII) can also be administered in mixture or in combination with agents useful to treat other disorders or maladies, such as steroids, membrane stablizers.

Pharmaceutical compositions, comprising active compounds(s) and/or prodrug(s) of formulae (I), (II), (III), (XI), (XII), and/or (XIII), used in the methods herein disclosed may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the active compounds(s) and/or prodrug(s) of formulae (I), (II), (III), (XI), (XII), and/or (XIII) into preparations which can be used pharmaceutically (see Remingtons's Pharmaceutical Sciences, 15^{th} Ed., Hoover, J. E. ed., Mack Publishing Co. (2003)

The active compounds(s) and/or prodrug(s) of formulae (I), (II), (III), (XI), (XII), and (XIII) can be formulated in the pharmaceutical compositions per se, or in the form of a hydrate, solvate, N-oxide or pharmaceutically acceptable salt. Such salts may be derived from acids or bases, as is well-known in the art. Typically, such salts are more soluble in aqueous solutions than the corresponding free acids and bases, but salts having lower solubility than the corresponding free acids and bases may also be formed. Such salts include salts suitable for pharmaceutical uses (“pharmaceutically-acceptable salts”), salts suitable for veterinary uses, etc. In some embodiments, the salt is a pharmaceutically acceptable salt. Generally, pharmaceutically acceptable salts are those salts that retain substantially one or more of the desired pharmacological activities of the parent compound and which are suitable for administration to humans.

Combination Therapies

The active compounds(s) and/or prodrug(s) of the present disclosure may be used alone, in combination, or as an adjunct to, or in conjunction with, other established antiproliferative therapies or with cytotoxic agents. Thus, the active compounds(s) and/or prodrug(s) of the present disclosure may be used with traditional cancer therapies, such as ionization radiation in the form of γ-rays and x-rays, delivered externally or internally by implantation of radioactive compounds, and as a follow-up to surgical removal of tumors. The active compounds(s) and/or prodrug(s) of the present disclosure and the other therapeutic agent may be administered simultaneously, sequentially, by the same route of administration, or by different routes.

Various chemotherapeutic agents may be used in combination with the active compounds(s) and/or prodrug(s) of formulae (I), (II), (III), (XI), (XII), and/or (XIII) provided herein to treat inhibit tumor cell proliferation and/or survival. These chemotherapeutic agents may be general cytotoxic agents or target a specific cellular molecule. Various classes of cancer chemotherapeutic agents include, among others, antimetabolites, agents that react with DNA (e.g., alkylating agents, coordination compounds, etc.), inhibitors of transcription enzymes, topoisomerase inhibitors, DNA minor-groove binding compounds, antimitotic agents (e.g., vinca alkyloids), antitumor antibiotics, hormones, and enzymes. Exemplary alkylating agents include, by way of example and not limitation, mechlorothamine, cyclophosphamide, ifosfamide, melphalan, chlorambucil, ethyleneimines, methylmelamines, alkyl sulfonates (e.g., busulfan), and carmustine. Exemplary antimetabolites include, by way of example and not limitation, folic acid analog methotrexate; pyrimidine analogs fluorouracil, cytosine arabinoside; and purine analogs mercaptopurine, thioguanine, and azathioprine. Exemplary vinca alkyloids include, by way of example and not limitation, vinblastine, vincristine, paclitaxel, and colchicine. Exemplary antitumor antibiotics include, by way of example and not limitation, actinomycin D, daunorubicin, and bleomycin. An exemplary enzyme effective as anti-neoplastic agent is L-asparaginase. Exemplary coordination compounds include, by way of example and not limitation, cisplatin and carboplatin. Exemplary hormones and hormone related compounds include, by way of example and not limitation, adrenocorticosteroids prednisone, and dexamethasone; aromatase inhibitors amino glutethimide, formestane, and anastrozole; progestin compounds hydroxyprogesteron caproate, medroxyprogesterone; and anti-estrogen compound tamoxifen. Exemplary topoisomerase inhibitors include, by way of example and not limitation, amsacrine (m-AMSA); mitoxantrone, topotecan, irinotecan, and camptothecin. Various derivative anti-neoplastic agents that combine more than one anticancer activity may be used. For instance, NSC290205 is a combination compound incorporating d-lactam derivative of androsterone and an alkylating agent based on N,N-bis(2-chloroethyl)aniline (Trafalis et al., 2005, Br. J. Haematol. 128(3):343-50).

These and other useful anti-cancer compounds are described in Merck Index, 13th Ed. (O'Neil M. J. et al., ed) Merck Publishing Group (2001) and Goodman and Gilmans The Pharmacological Basis of Therapeutics, 10th Edition, Hardman, J. G. and Limbird, L. E. eds., pg. 1381-1287, McGraw Hill, (1996), both of which are incorporated by reference herein.

Additional antiproliferative compounds useful in combination with the active compounds(s) and/or prodrug(s) of formulae (I), (II), (III), (XI), (XII), and/or (XIII) described herein include, by way of example and not limitation, antibodies directed against growth factor receptors (e.g., anti-Her2); cytokines such as interferon-α and interferon-γ, interleukin-2, interleukin-6, IGF-I, and GM-CSF; and antibodies for cell surface markers (e.g., anti-CTLA-4. anti-CD20 (rituximab); anti-CD33). When antibodies against cell surface markers are used, a chemotherapeutic agent may be conjugated to it for delivering the agent to the tumor cell. Suitable conjugates include radioactive compounds (e.g., radioactive metal bound to a antibody conjugated chelator), cytotoxic compounds, and drug activating enzymes (e.g., allinase, peptidases, esterases, catalytic antibodies, etc.) (see, e.g., Arditti et al., 2005, Mol. Cancer. Therap. 4(2):325-331; U.S. Pat. No. 6,258,360; incorporated herein by reference)

In some embodiments, the active compounds(s) and/or prodrug(s) of formulae (I), (II), (III), (XI), (XII), and/or (XIII) provided herein may be used with a kinase inhibitor that targets an oncogenic kinase. In some embodiments, the kinase inhibitor is an inhibitor of Abl kinase. For example, chronic myelogenous leukemia is a myeloid neoplasm characterized by malignant proliferation of leukemic stem cells in the bone marrow. The majority of chronic myelogenous leukemia is associated with a cytogenetic abnormality defined by a reciprocal translocation t(9; 22)(q34; q11). This chromosomal aberration results in generation of a BCR/ABL fusion protein with activated kinase activity. Inhibitors of the fusion protein kinase activity are effective in treating chronic myelogenous leukemia although resistant forms may develop upon continued treatment. Use of the active compounds(s) and/or prodrug(s) of formulae (I), (II), (III), (XI), (XII), and/or (XIII) provided herein in combination of Abl kinase inhibitors may lessen the chances of resistant cells by targeting a cellular process different than that targeted by the kinase inhibitor alone. An exemplary Abl kinase inhibitor is 2-phenylaminopyrimidine, also known as imatinib mesylate and GLEEVEC®. Thus, in some embodiments, the active compounds(s) and/or prodrug(s) of formulae (I), (II), (III), (XI), (XII), and/or (XIII) provided herein may be used in combination with Abl kinase inhibitor 2-phenylaminopyrimidine and its derivatives. In other embodiments, the kinase inhibitor may be pyridol[2-3-d]pyrimidine and its derivatives, which was originally identified as inhibitors of Src kinase. In yet further embodiments, the kinase inhibitor is tyrphostins and its derivatives (e.g., adaphostin) which affects the association of the kinase with its substrates. Other kinase inhibitor compounds will be apparent to the skilled artisan.

As further described herein, the administration of other chemotherapeutic agents may be done in the form of a composition, or administered adjunctively in combination with the active compounds(s) and/or prodrug(s) of formulae (I), (II), (III), (XI), (XII), and/or (XIII) provided herein. When provided adjunctively, the chemotherapeutic agents may be administered simultaneously with or sequentially with administration of the active compounds(s) and/or prodrug(s) of formulae (I), (II), (III), (XI), (XII), and/or (XIII). Such chemotherapeutic agents are, e.g., HDAC inhibitors (e.g., MGCD0103 and vorinostat), HSP 90 inhibitors (e.g., 17-AAG), BCL-2 inhibitors, thalidomide, lenalidomide, mTOR inhibitors (e.g., rapamycin, CCI-779), sorafenib, doxorubicine, gemcitabine, dexamethasone, melphalan, proteasome inhibitors (e.g., bortezomib, NPI052), monoclonal antibodies (e.g., rituximab, anti-TRAIL deat receptor antibodies), and the like.

### EXAMPLES

### Example 1

### Synthesis of Prodrug Compounds

### 1. N4-(2,2-dimethyl-4-[(di-tert-butyl phosphonoxy)methyl]-3-oxo-5-pyrido[1,4]oxazin-6-yl)-5-fluoro-N2-(3,4,5-trimethoxyphenyl)-2,4-pyrimidinediamine (Compound 3)

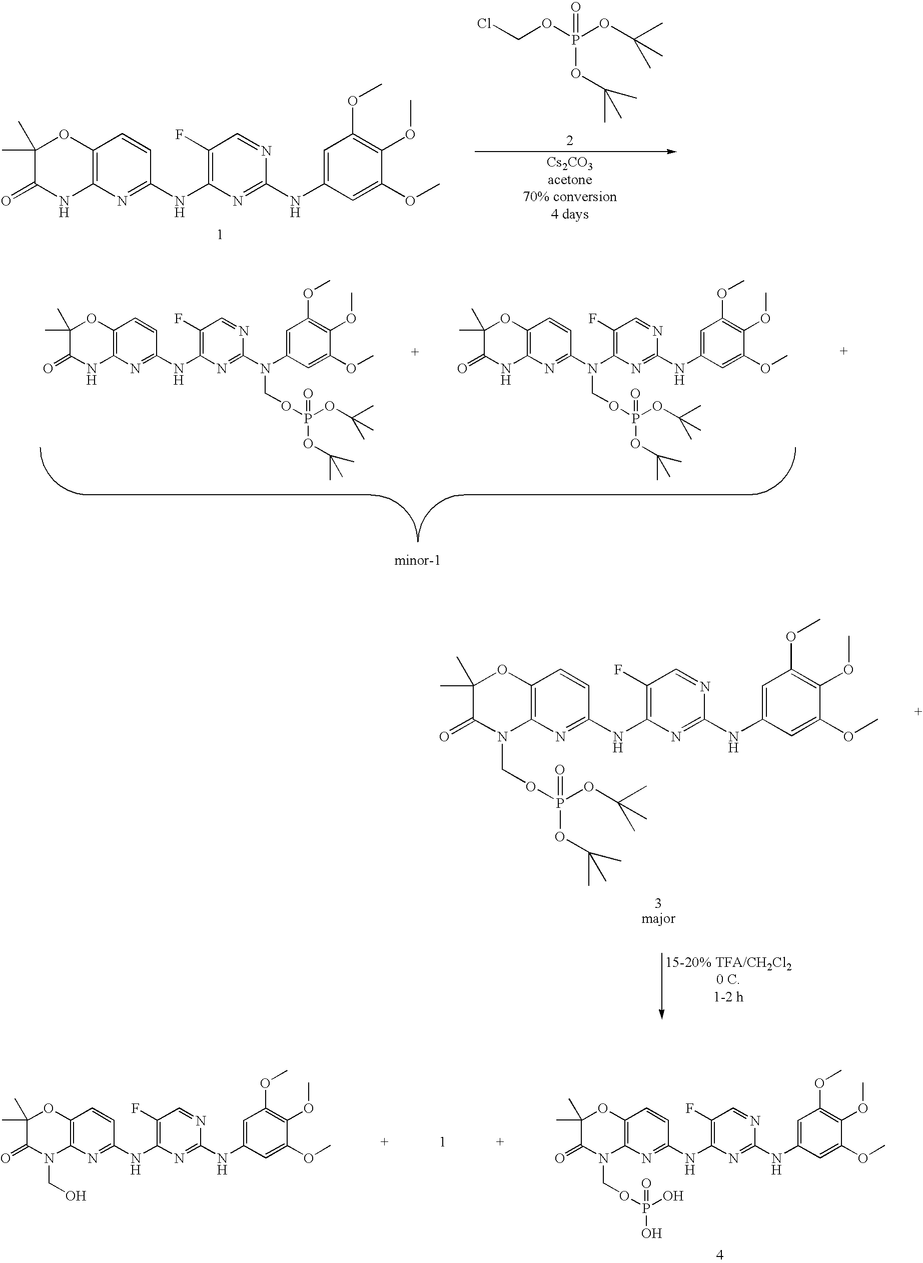

N4-(2,2-dimethyl-3-oxo-4H-5-pyrido[1,4]oxazin-6-yl)-5-fluoro-N2-(3,4,5-trimethoxyphenyl)-2,4-pyrimidinediamine (1, 1.0 g, 2.12 mmol), Cs₂CO₃ (1.0 g, 3.07 mmol) and di-tert-butyl chloromethyl phosphate (2, 0.67 g, 2.59 mmol) in acetone (20 mL) was stirred at room temperature under nitrogen atmosphere. Progress of the reaction was monitored by LC/MS. Crude reaction mixture displayed three product peaks with close retention times with M⁺+H 693 (minor-1), 693 (major; 3) and 477 (minor-2) besides starting material (Compound 1). Upon stirring the contents for 4 days (70% consumption), the reaction mixture was concentrated and diluted with water. The resultant pale yellow precipitate formed was collected by filtration and dried. The crude solid was purified by silica gel (pretreated with 10% NEt₃/CH₂Cl₂ followed by eluting with hexanes) column chromatography by gradient elution with 70% EtOAc/hexanes-100% EtOAc). The fractions containing Compound 1 and M⁺+H 693 were collected and concentrated. The resulting crude white solid was subjected to repurification in the similar manner as described previously but by eluting with 30%-50%-75%-100% EtOAc/hexanes. The major product peak with M⁺+H 693 was collected as a white solid (270 mg, 18%) and was characterized as N4-(2,2-dimethyl-4-[(di-tert-butylphosphonoxy)methyl]-3-oxo-5-pyrido[1,4]oxazin-6-yl)-5-fluoro-N2-(3,4,5-trimethoxyphenyl)-2,4-pyrimidinediamine (Compound 3). ¹H NMR (DMSO-d6): δ 9.21 (s, 1H), 9.17 (s, 1H), 8.16 (d, 1H, J=2.6 Hz), 7.76 (d, 1H, J=8.5 Hz), 7.44 (d, 1H, J=8.5 Hz), 7.02 (s, 2H), 5.78 (d, 1H, J³_{PH}=6.1 Hz), 3.64 (s, 6H), 3.58 (s, 3H), 1.45 (s, 6H), 1.33 (s, 9H). LCMS: ret. time: 14.70 min.; purity: 95%; MS (m/e): 693 (MH⁺). ³¹P NMR (DMSO-d6): −11.36.

### 2. N4-(2,2-dimethyl-4-[(dihydrogen phosphonoxy)methyl]-3-oxo-5-pyrido[1,4]oxazin-6-yl)-5-fluoro-N2-(3,4,5-trimethoxyphenyl)-2,4-pyrimidinediamine (Compound 4)

Trifluoroacetic acid (1.5 mL) was added dropwise as a neat for 5 min to N4-(2,2-dimethyl-4-[(di-tert-butylphosphonoxy)methyl]-3-oxo-5-pyrido[1,4]oxazin-6-yl)-5-fluoro-N-2-(3,4,5-trimethoxyphenyl)-2,4-pyrimidinediamine (Compound 3, 120 mg, 0.173 mmol) dissolved in CH₂Cl₂ (10 mL) at 0° C. under nitrogen atmosphere. The contents were allowed to stir for 1.5 h. Progress of the reaction mixture was monitored by LC/MS. After complete consumption of the starting material, reaction mixture was concentrated, dried and triturated with ether. The ethereal layer was decanted and dried to provide the crude solid. LC/MS analysis of the crude displayed three peaks with M⁺+H 581, 471 and 501. The peak corresponding to M⁺+H 581 was collected by preparative HPLC chromatographic purification. The fractions were lyophilised and dried to provide 53 mg (52%) of off white fluffy solid and characterized as N4-(2,2-dimethyl-4-[(dihydrogenphosphonoxy)methyl]-3-oxo-5-pyrido[1,4]oxazin-6-yl)-5-fluoro-N2-(3,4,5-trimethoxyphenyl)-2,4-pyrimidinediamine (Compound 4). ¹H NMR (DMSO-d6): δ 9.21 (br s, 2H), 8.16 (d, 1H, J=2.6 Hz), 7.93 (d, 1H, J=8.5 Hz), 7.39 (d, 1H, J=8.5 Hz), 7.05 (s, 2H), 5.79 (d, 1H, J³_{PH}=6.6 Hz), 3.67 (s, 6H), 3.59 (s, 3H), 1.44 (s, 6H). LCMS: ret. time: 8.52 min.; purity: 95%; MS (m/e): 581 (MH⁺). ³¹P NMR (DMSO-d6): −2.17.

### 3. Alternative Synthesis of Prodrug Compound 4

An alternative method of synthesizing prodrug Compound 4 which alleviates the need for column chromatography and HPLC purification is provided below.

### 3a. Synthesis of N4-(2,2-dimethyl-4-[(di-tert-butyl phosphonoxy)methyl]-3-oxo-5-pyrido[1,4]oxazin-6-yl)-5-fluoro-N2-(3,4,5-trimethoxyphenyl)-2,4-pyrimidinediamine (Compound 3)

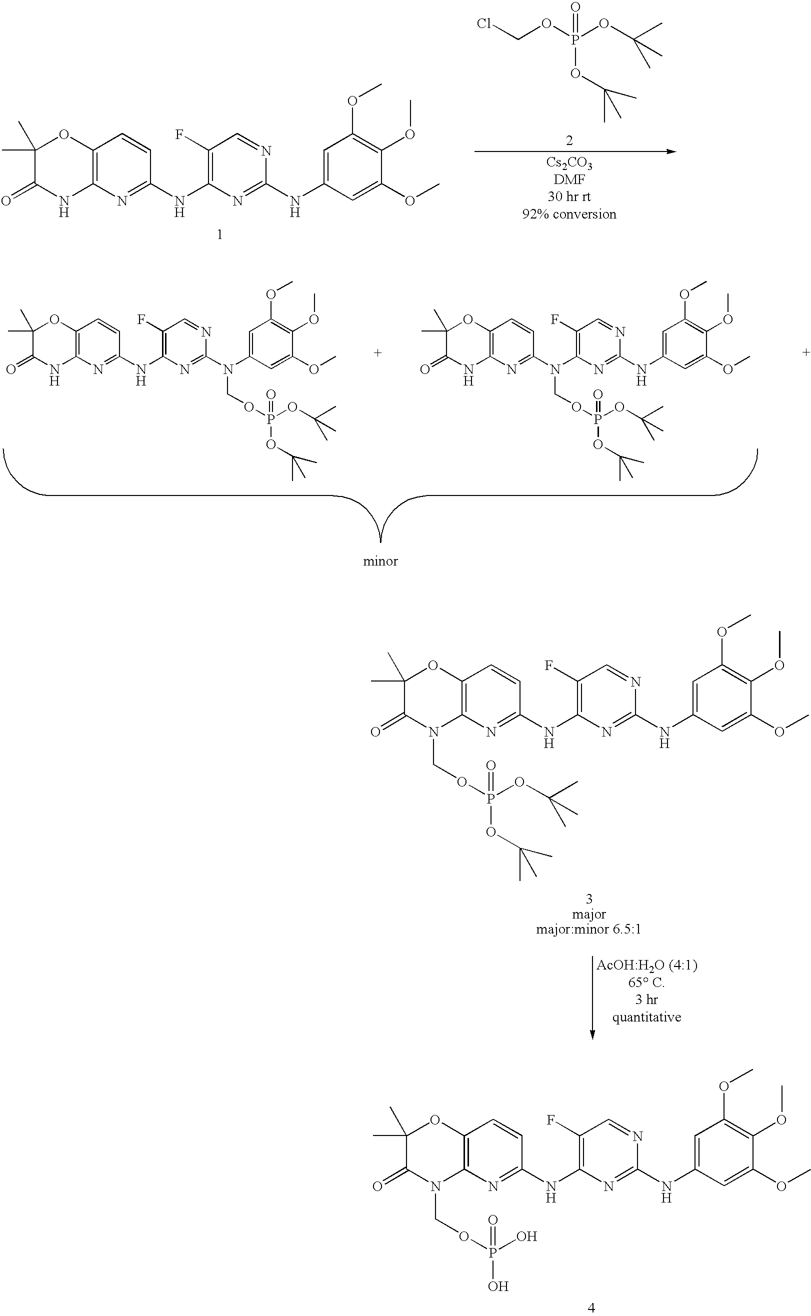

N4-(2,2-dimethyl-3-oxo-4H-5-pyrido[1,4]oxazin-6-yl)-5-fluoro-N2-(3,4,5-trimethoxyphenyl)-2,4-pyrimidinediamine (Compound 1, 19.73 g, 41.97 mmol), Cs₂CO₃ (15.04 g, 46.16 mmol) and di-tert-butyl chloromethyl phosphate (13.0 g, 50.38 mmol) in DMF (100 mL) was stirred at room temperature under nitrogen atmosphere. Progress of the reaction was monitored by in process LC/MS. Crude reaction mixture displayed two product peaks (ratio 1:6.5) with close retention times displaying M⁺+H 693 (minor) and 693 (major) besides starting material (Compound 1). Initial yellow reaction mixture turned to olive green as the reaction progressed. Workup was carried out as follows
1). Upon stirring the contents for 30 h (92% consumption), reaction mixture was poured onto ice-water (400 mL) and stirred the contents by adding brine solution (200 mL). Fine yellow tan solid formed was filtered, washed with water and dried overnight.
2). The solid (35 g) was dissolved in MTBE (500 mL) and washed with water (400 mL). Aqueous layer was extracted with MTBE (2×350 mL) till the absence of UV on TLC. Combined organic layers were dried over anhydrous Na₂SO₄ and decanted. Note: step 2 can be done directly, however, DMF extraction back into solution leads to difficulty in the crystallization step.
3). The dark red clear solution was subjected to 10 g of activated charcoal treatment, heated to boil and filtered.
4). The dark red clear solution was concentrated by normal heating to 400 mL of its volume and left for crystallization. The solid crystallized as granules was filtered, crushed the granules to powder, washed with MTBE (400 mL) and dried under high vacuum. See step 7 for the workup of mother liquor. Weight of the solid: 17 g; purity: 90% (Compound 3), 6.26% (Compound 1), 1.8% (minor M+ 693).
5). At this stage solid was taken in 500 mL of ethyl ether and heated to boil. Cooled and filtered to remove undissolved material. Filtrate was concentrated.
6). Above concentrate was subjected to crystallization in MTBE (300 mL). The white solid formed was filtered, washed with MTBE (100 mL) and dried under high vacuum to provide the desired N4-(2,2-dimethyl-4-[(di-tert-butyl phosphonoxy)methyl]-3-oxo-5-pyrido[1,4]oxazin-6-yl)-5-fluoro-N2-(3,4,5-trimethoxyphenyl)-2,4-pyrimidinediamine (Compound 3) in 97% purity. ¹H NMR (DMSO-d6): δ 9.21 (s, 1H), 9.17 (s, 1H), 8.16 (d, 1H, J=2.6 Hz), 7.76 (d, 1H, J=8.5 Hz), 7.44 (d, 1H, J=8.5 Hz), 7.02 (s, 2H), 5.78 (d, 1H, J³_{PH}=6.1 Hz), 3.64 (s, 6H), 3.58 (s, 3H), 1.45 (s, 6H), 1.33 (s, 9H). LCMS: ret. time: 14.70 min.; purity: 95%; MS (m/e): 693 (MH⁺). ³¹P NMR (DMSO-d6): −11.36. Weight of the solid: 15.64 g (yield: 55%); purity: 97% (Compound 3), 3% (Compound 1).
7). The mother liquor was concentrated and steps 5 and 6 were repeated to provide Compound 3.

### 3b. Synthesis of N4-(2,2-dimethyl-4-[(dihydrogenphosphonoxy)methyl]-3-oxo-5-pyrido[1,4]oxazin-6-yl)-5-fluoro-N2-(3,4,5-trimethoxyphenyl)-2,4-pyrimidinediamine (Compound 4)

N4-(2,2-dimethyl-4-[(di-tert-butylphosphonoxy)methyl]-3-oxo-5-pyrido[1,4]oxazin-6-yl)-5-fluoro-N2-(3,4,5-trimethoxyphenyl)-2,4-pyrimidinediamine (Compound 3); (15.0 g, 21.67 mmol) dissolved in AcOH:H₂O (225 mL, 4:1) was heated at 65° C. (oil bath temp). The progress of the reaction was monitored by in process LC/MS. The reaction mixture transformed to faint tan white solid after 1 h of heating. At this point most of Compound 3 converted to mono des t-butyl product. After 3 h of heating, consumption of SM and complete conversion of intermediate (mono des t-butylated) to product was observed.

Reaction mixture was cooled, poured onto ice-water (200 mL), stirred for 20 min and filtered. The clear white filter cake was washed with water (600 mL) and acetone (200 mL) successively, dried for 2 h followed by drying under high vacuum over P₂O₅ in a desiccator. Weight of the solid: 12.70 g; purity: 97% (Compound 3) and 3% (Compound 1) ¹H NMR indicated acetic acid presence (1:1)

To remove acetic acid, the solid was taken in acetonitrile (300 mL) and concentrated by rotovap vacuum. This process was repeated 2 times with acetonitrile and toluene (3×300 mL). The solid obtained was dried under high vacuum at 50° C.

Finally, the solid was taken in acetone (400 mL), filtered and dried to provide solid N4-(2,2-dimethyl-4-[(dihydrogenphosphonoxy)methyl]-3-oxo-5-pyrido[1,4]oxazin-6-yl)-5-fluoro-N2-(3,4,5-trimethoxyphenyl)-2,4-pyrimidinediamine (Compound 4). ¹H NMR (DMSO-d6): δ 9.21 (br s, 2H), 8.16 (d, 1H, J=2.6 Hz), 7.93 (d, 1H, J=8.5 Hz), 7.39 (d, 1H, J=8.5 Hz), 7.05 (s, 2H), 5.79 (d, 1H, J³_{PH}=6.6 Hz), 3.67 (s, 6H), 3.59 (s, 3H), 1.44 (s, 6H). LCMS: ret. time: 8.52 min.; purity: 95%; MS (m/e): 581 (MH⁺). ³¹P NMR (DMSO-d6): −2.17.

### 4. Synthesis of N4-(2,2-dimethyl-4-[(dihydrogen phosphonoxy)methyl]-3-oxo-5-pyrido[1,4]oxazin-6-yl)-5-fluoro-N2-(3,4,5-trimethoxyphenyl)-2,4-pyrimidinediamine mono calcium salt (Prodrug Salt 6)

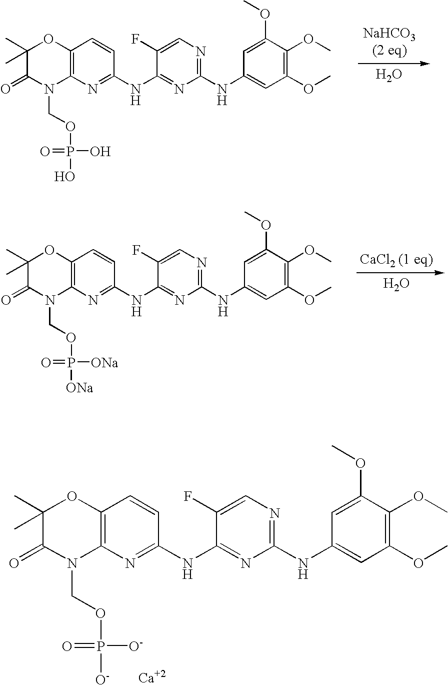

Aqueous (10 mL) NaHCO3 (0.17 g, 2.02 mmol) solution was added dropwise to a suspension of N4-(2,2-dimethyl-4-[(dihydrogenphosphonoxy)methyl]-3-oxo-5-pyrido[1,4]oxazin-6-yl)-5-fluoro-N2-(3,4,5-trimethoxyphenyl)-2,4-pyrimidinediamine (0.5 g, 0.86 mmol) in water (5 mL) at room temperature while stirring the contents. The clear solution formed was treated with aqueous (10 mL) CaCl₂ (0.11 g in 10 mL water, 0.99 mmol) in a dropwise manner at room temperature. The addition resulted in the precipitation of a white solid from reaction mixture. Upon completion of addition, the contents were stirred for a period of 30 min, filtered, washed with water (40 mL) and dried. The clear white solid was taken in water (30 mL) and heated on a stir plate to boil. The solution was cooled, filtered and dried. The white solid collected and further dried under high vacuo at 80° C. for 32 h to provide 0.41 g (83%) of solid N4-(2,2-dimethyl-4-[(dihydrogen phosphonoxy)methyl]-3-oxo-5-pyrido[1,4]oxazin-6-yl)-5-fluoro-N2-(3,4,5-trimethoxyphenyl)-2,4-pyrimidinediamine mono calcium salt (Prodrug Salt 6). Ca(OAc)₂ may also used in place Of CaCl₂ in this preparation.

### 5. Synthesis of N4-(2,2-dimethyl-4-[(dihydrogen phosphonoxy)methyl]-3-oxo-5-pyrido[1,4]oxazin-6-yl)-5-fluoro-N2-(3,4,5-trimethoxyphenyl)-2,4-pyrimidinediamine disodium salt hexahydrate and monosodium salt hydrate

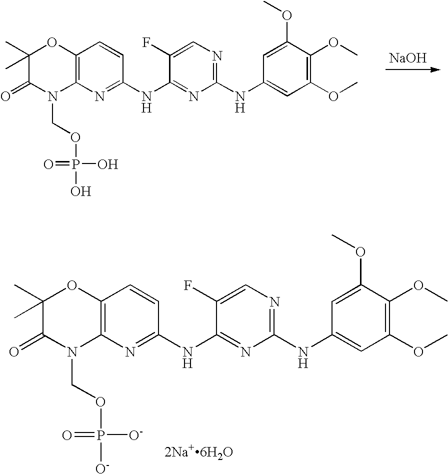

A round-bottomed flask was charged with 10.00 g N4-(2,2-dimethyl-4-[(dihydrogen phosphonoxy)methyl]-3-oxo-5-pyrido[1,4]oxazin-6-yl)-5-fluoro-N2-(3,4,5-trimethoxyphenyl)-2,4-pyrimidinediamine (Compound 4) and 140 mL water into a round bottom flask to form a slurry having a pH between 3.6 and 3.7. The pH was adjusted to in the range of 9.3 to 10.3 by addition of 1 M aqueous NaOH, initially forming a turbid solution, which returned to a suspension upon prolonged stirring. The mixture was heated at reflux, then the turbid solution was hot filtered through filter paper. The solid collected in the filter paper was rinsed with 10 mL hot water. Isopropanol (75 mL) was added to the filtrate, yielding a clear solution, which was allowed to cool to room temperature over about 1.5 hours with stirring, during which time a solid precipitated. The precipitate was collected by filtration, rinsed with 47 mL isopropanol, and taken up in 73 mL acetone to form a slurry, which was stirred for 1.5 hours at room temperature. The solid was again collected by filtration and rinsed with 18 mL acetone, then dried at about 40° C. under vacuum until substantially all isopropanol and acetone was removed (i.e., below 0.5 wt % each). The product was exposed to air at about 40% relative humidity and room temperature until the water content stabilized at about 15% by Karl Fisher titration, yielding 8.18 g of the title compound. ¹H NMR (D₂O): δ 7.67 (d, 1H, J=3.8 Hz), 7.49 (d, 1H, J=8.8 Hz), 6.87 (d, 1H, J=8.8 Hz), 6.50 (s, 2H), 5.52 (d, 1H, J³_{PH}=2.0 Hz), 3.53 (s, 3H), 3.47 (s, 6H), 1.32 (s, 6H). ³¹P NMR (D₂O): 2.75. The prodrug salt hydrate was obtained as a pure-white, highly crystalline material. Microscopic investigation indicated that the crystallites are plate-like with a particle size of less than 10 μm. Polarized light microscopy revealed birefringence corroborating the crystalline nature of the hydrate.

The monosodium salt can be prepared from N4-(2,2-dimethyl-4-[(dihydrogen phosphonoxy)methyl]-3-oxo-5-pyrido[1,4]oxazin-6-yl)-5-fluoro-N2-(3,4,5-trimethoxyphenyl)-2,4-pyrimidinediamine and sodium hydroxide by a proper pH control; pH of 5-5.5 results in predominantly the formation of monosodium salt.

### 6. Preparation of N4-(2,2-dimethyl-4-[(dihydrogenphosphonoxy)methyl]-3-oxo-5-pyrido[1,4]oxazin-6-yl)-5-fluoro-N2-(3,4,5-trimethoxyphenyl)-2,4-pyrimidinediamine dipotassium salt

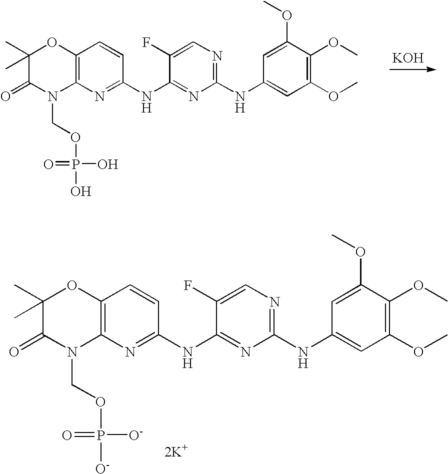

A suspension of N4-(2,2-dimethyl-4-[(dihydrogen phosphonoxy)methyl]-3-oxo-5-pyrido[1,4]oxazin-6-yl)-5-fluoro-N2-(3,4,5-trimethoxyphenyl)-2,4-pyrimidinediamine, acetic acid complex (1.0 g, 1.56 mmol) in water (15 mL) was heated at 70° C. (oil bath temp) for 10 min (pH=2.9). To the above stirring suspension, aqueous KOH (2.1 M, 1.5 ml) was added dropwise and the pH was observed as 5.9. At this point 2.5 M aqueous KOH was added dropwise while monitoring the pH. When the pH reached to 10.5 (after 0.95 mL), addition was stopped and the clear solution stirred at the same temperature for 15 min. The warm solution was filtered into a conical flask and washed the filter paper with water to a combined volume of 45 mL. The filtrate was transferred onto a hot plate and isopropanol (175 mL) was added portionwise to the hot solution, until the turbidity persisted upon heating. Water was then added dropwise until the solution was just clear at its boiling point. The conical flask was removed from the hot plate and allowed to cool to room temperature. A crystalline solid formed, which was collected by suction filtration, washed with minimum amount of isopropanol and dried for 30 min. The resultant white solid was dried under vacuum overnight at 70° C., yielding N4-(2,2-dimethyl-4-[(dihydrogenphosphonoxy)methyl]-3-oxo-5-pyrido[1,4]oxazin-6-yl)-5-fluoro-N2-(3,4,5-trimethoxyphenyl)-2,4-pyrimidinediamine dipotassium salt, (0.95 g, 1.44 mmol, 92%, 99% pure) ¹H NMR (D₂O): δ 7.68 (d, 1H, J=3.8 Hz), 7.49 (d, 1H, J=8.8 Hz), 6.87 (d, 1H, J=8.8 Hz), 6.51 (s, 2H), 5.52 (d, 1H, J³_{PH}=2.0 Hz), 3.54 (s, 3H), 3.48 (s, 6H), 1.32 (s, 6H). ³¹P NMR (D₂O): 2.7.

### 7. Preparation of N4-(2,2-dimethyl-4-[(dihydrogen phosphonoxy)methyl]-3-oxo-5-pyrido[1,4]oxazin-6-yl)-5-fluoro-N2-(3,4,5-trimethoxyphenyl)-2,4-pyrimidinediamine di-L-Arginine Salt

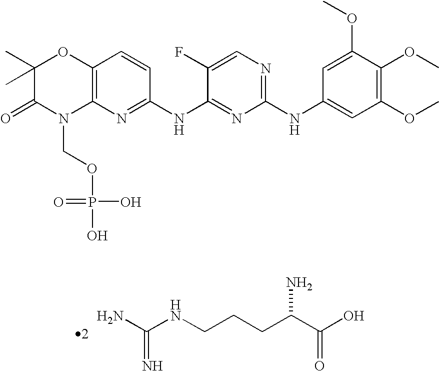

A suspension of N4-(2,2-dimethyl-4-[(dihydrogen phosphonoxy)methyl]-3-oxo-5-pyrido[1,4]oxazin-6-yl)-5-fluoro-N2-(3,4,5-trimethoxyphenyl)-2,4-pyrimidinediamine (0.25 g, 0.43 mmol) and L-arginine (0.15 g, 0.86 mmol) in EtOH (15 mL) was heated at 90° C. (oil bath temp) for 10 min. Water (7.5 mL) was added dropwise to the hot stirring suspension until it became a clear solution. After 1 h of heating with stirring, the hot solution was filtered into an Erlenmeyer flask. The filtrate in the Erlenmeyer flask was brought to boiling on a hot plate, the allowed to cool to room temperature. A solid formed, which was collected by gravity filtration and dried under vacuum overnight at 80° C. to form the subject prodrug salt hydrate (0.28 g, 0.3 mmol, 69%). ¹H NMR (D₂O): δ 7.64 (d, 1H, J=3.5 Hz), 7.42 (d, 1H, J=8.8 Hz), 6.80 (d, 1H, J=8.8 Hz), 6.45 (s, 2H), 5.53 (d, 1H, J³_{PH}=2.8 Hz), 3.57 (t, 2H, J=6.0 Hz), 3.51 (s, 3H), 3.44 (s, 6H), 3.01 (t, 4H, J=6.5 Hz), 1.74-1.69 (m, 4H), 1.55-1.46 (m, 4H), 1.30 (s, 6H). ³¹P NMR (D₂O): 2.56.

### 8. Preparation of N4-(2,2-dimethyl-4-[(dihydrogen phosphonoxy)methyl]-3-oxo-5-pyrido[1,4]oxazin-6-yl)-5-fluoro-N2-(3,4,5-trimethoxyphenyl)-2,4-pyrimidinediamine di-L-lysine Salt

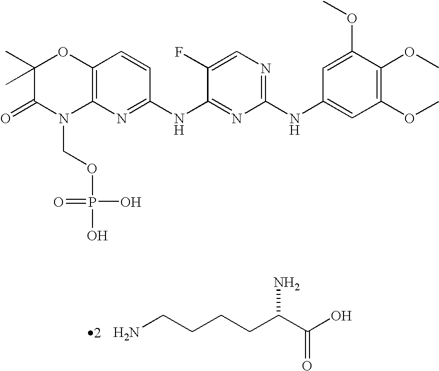

A suspension of N4-(2,2-dimethyl-4-[(dihydrogen phosphonoxy)methyl]-3-oxo-5-pyrido[1,4]oxazin-6-yl)-5-fluoro-N2-(3,4,5-trimethoxyphenyl)-2,4-pyrimidinediamine (0.25 g, 0.43 mmol) and L-lysine (0.125 g, 0.86 mmol) in EtOH (15 mL) was heated at 90° C. (oil bath temp) for 10 min. Water (4.5 mL) was added dropwise to the hot stirring suspension until it formed a clear solution. After 1 h of heating and stirring, the reaction mixture filtered, cooled and concentrated under vacuum. Precipitation of the crude concentrate was observed upon addition of EtOH (5 mL). The resultant solid was stirred overnight at room temperature in t-BuOMe after concentration of the mixture. The white solid was collected by gravity filtration and dried under vacuum overnight at 80° C. (0.32 g, 83%). ¹H NMR (D₂O): δ 7.67 (d, 1H, J=3.8 Hz), 7.47 (d, 1H, J=8.8 Hz), 6.84 (d, 1H, J=8.8 Hz), 6.48 (s, 2H), 5.54 (d, 1H, J³_{PH}=3.5 Hz), 3.57 (t, 2H, J=6.0 Hz), 3.51 (s, 3H), 3.44 (s, 6H), 2.86 (t, 4H, J=6.7 Hz), 1.77-1.70 (m, 4H), 1.62-1.52 (app q, 4H, J=6.3 Hz), 1.38-1.26 (m, 10H). ³¹P NMR (D₂O): 2.59.

### 9. Synthesis of N4-(2,2-dimethyl-4-[(dihydrogen phosphonoxy)methyl]-3-oxo-5-pyrido[1,4]oxazin-6-yl)-5-fluoro-N2-(3,4,5-trimethoxyphenyl)-2,4-pyrimidinediamine mono magnesium salt

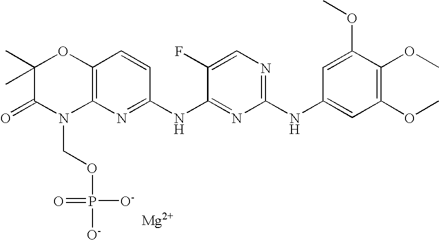

A suspension of N4-(2,2-dimethyl-4-[(dihydrogen phosphonoxy)methyl]-3-oxo-5-pyrido[1,4]oxazin-6-yl)-5-fluoro-N2-(3,4,5-trimethoxyphenyl)-2,4-pyrimidinediamine dipotassium salt (0.5 g, 0.76 mmol) in 10 mL water was placed onto preheated oil bath at 80° C. and stirred till the suspension formed a clear solution. The hot solution was filtered, and the filter paper was washed with another 10 mL of water. The clear filtrate was heated while stirring at 80° C. MgCl₂ (0.076 g, 0.8 mmol) was dissolved in water (10 mL), filtered into a flask through a filter column (rinsing with 10 mL water), and heated at 90° C. (pH=7.52). The preheated dipotassium salt solution was added dropwise to the above MgCl₂ solution for 15 min while stirring the contents. The initial white frothing suspension formed slowly turned to clear white solid upon heating the contents at 80° C. for 1.5 h (pH=6.3-6.7). The solid was collected by suction filtration and washed with water until there was no chloride ion was detected (AgNO₃ test). The solid was suction dried for 2 h, then by vacuum dried at 70° C. overnight to provide N4-(2,2-dimethyl-4-[(dihydrogenphosphonoxy)methyl]-3-oxo-5-pyrido[1,4]oxazin-6-yl)-5-fluoro-N2-(3,4,5-trimethoxyphenyl)-2,4-pyrimidinediamine mono magnesium salt (0.43 mg, 93%).

### 10. Synthesis of N2-Chlorocarbonyl-N4-(2,2-dimethyl-3-oxo-4H-5-pyrido[1,4]oxazin-6-yl)-5-fluoro-N2-(3,4,5-trimethoxyphenyl)-2,4-pyrimidinediamine

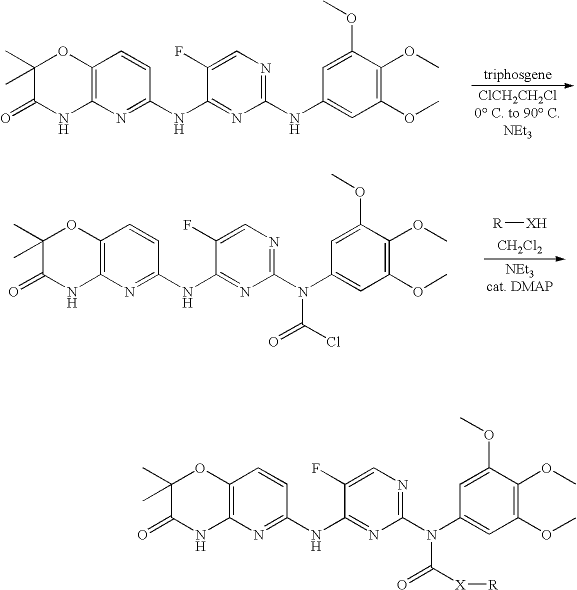

To the (pale yellow) stirring mixture of N4-(2,2-dimethyl-3-oxo-4H-5-pyrido[1,4]oxazin-6-yl)-5-fluoro-N2-(3,4,5-trimethoxyphenyl)-2,4-pyrimidinediamine (2.5 g, 5.31 mmol) and triphosgene (1.67 g, 5.62 mmol) in dicholoroethane (20 mL) at 0° C., NEt₃ (1.08 g, 1.5 mL, 10.76 mmol) in dichloroethane (10 mL) was added dropwise under nitrogen atmosphere for 10 min. The (orange) reaction mixture was allowed to stir for 15 min at 0° C. followed by refluxing at 90° C. overnight. The heterogeneous (tan orange) reaction mixture was cooled to room temperature. The reaction mixture was diluted with EtOAc (75 mL). Precipitated white solid formed was filtered. The white solid was collected, treated with water, filtered and dried to provide N2-chlorocarbonyl-N4-(2,2-dimethyl-3-oxo-4H-5-pyrido[1,4]oxazin-6-yl)-5-fluoro-N2-(3,4,5-trimethoxyphenyl)-2,4-pyrimidinediamine (1.75 g, 61%). ¹H NMR (DMSO-d₆): δ 11.08 (s, 1H), 9.97 (s, 1H), 8.44 (d, 1H, J=3.2 Hz), 7.35 (d, 1H, J=8.5 Hz), 7.24 (d, 1H, J=8.5 Hz), 6.77 (s, 1H), 3.72 (s, 6H), 3.66 (s, 3H), 1.40 (s, 6H). LCMS: ret. time: 12.53 min.; purity: 95%; MS (m/e): 534 (MH⁺).

### General Procedure for the Preparation of Ureas and Carbamates:

To N2-chlorocarbonyl-N4-(2,2-dimethyl-3-oxo-4H-5-pyrido[1,4]oxazin-6-yl)-5-fluoro-N2-(3,4,5-trimethoxyphenyl)-2,4-pyrimidinediamine (1 eq) in dry CH₂Cl₂ (4.8 mL/mmol), was added, the amine (for ureas) or alcohol (for carbamates) (2 eq), NEt₃ (7 eq) and DMAP (0.1 eq) successively under nitrogen atmosphere at room temperature. Contents were allowed to stir at room temperature and progress of the reaction mixture was monitored by LC/MS. Reaction mixture was concentrated upon consumption of carbamoylchloride. The crude concentrate was treated with aq. NaHCO₃ and the resulting solid precipitated was filtered, washed with water, dried and purified by either silica gel column chromatography or preparative HPLC.

### 11. Synthesis of N4-(2,2-Dimethyl-3-oxo-4H-5-pyrido[1,4]oxazin-6-yl)-5-fluoro-N2-[[[2-(4-morpholin-4-yl)ethyl]amino]carbonyl]-N2-(3,4,5-trimethoxyphenyl)-2,4-pyrimidinediamine

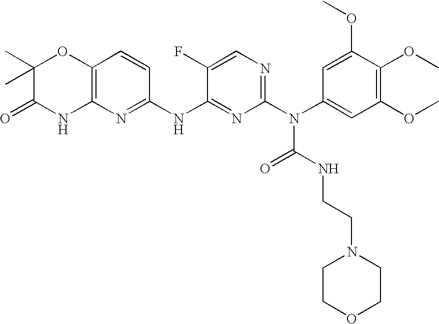

N4-(2,2-Dimethyl-3-oxo-4H-5-pyrido[1,4]oxazin-6-yl)-5-fluoro-N2-[[[2-(morpholin-4-yl)ethyl]amino]carbonyl]-N2-(3,4,5-trimethoxyphenyl)-2,4-pyrimidinediamine was prepared, according to the general procedure as described above, from N2-chlorocarbonyl-N4-(2,2-dimethyl-3-oxo-4H-5-pyrido[1,4]oxazin-6-yl)-5-fluoro-N2-(3,4,5-trimethoxyphenyl)-2,4-pyrimidinediamine and 4-(2-aminoethyl)morpholine. ¹H NMR (CDCl₃): δ 10.45 (s, 1H), 10.26 (t, 1H, J=4.7 Hz), 8.90 (s, 1H), 8.04 (d, 2H, J=2.9 Hz), 6.98 (d, 1H, J=8.8 Hz), 6.92 (d, 1H J=8.8 Hz), 6.51 (s, 2H), 3.93 (s, 3H), 3.80 (s, 6H), 3.77 (t, 4H, J=4.7 Hz), 3.54 (app qt, 2H, J=6.1 Hz), 2.64 (t, 2H, J=6.1 Hz), 2.57 (m, 4H), 1.51 (s, 6H). LCMS: ret. time: 8.32 min.; purity: 95%; MS (m/e): 627 (MH⁺).

### 12. Synthesis of N2-[[[(1S)-1-(t-Butoxycarbonyl)-2-methylpropyl]amino]-carbonyl]-N4-(2,2-dimethyl-3-oxo-4H-5-pyrido[1,4]oxazin-6-yl)-5-fluoro-N2-(3,4,5-trimethoxyphenyl)-2,4-pyrimidinediamine

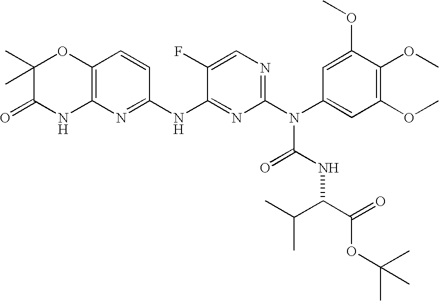

N2-[[[(1S)-1-(t-Butoxycarbonyl)-2-methylpropyl]amino]carbonyl]-N4-(2,2-dimethyl-3-oxo-4H-5-pyrido[1,4]oxazin-6-yl)-5-fluoro-N2-(3,4,5-trimethoxyphenyl)-2,4-pyrimidinediamine was prepared from N2-chlorocarbonyl-N4-(2,2-dimethyl-3-oxo-4H-5-pyrido[1,4]oxazin-6-yl)-5-fluoro-N2-(3,4,5-trimethoxyphenyl)-2,4-pyrimidinediamine and L-valine t-butyl ester hydrochloride in the similar manner as described in the general procedure. ¹H NMR (DMSO-d6): δ 10.97 (s, 1H), 10.45 (d, 1H, J=7.6 Hz), 8.35 (d, 1H, J=3.5 Hz), 6.75 (d, 1H, J=8.8 Hz), 6.71 (d, 1H, J=8.8 Hz), 6.52 (s, 2H), 4.15 (dd, 1H, J=4.7 and 6.7 Hz), 3.71 (s, 3H), 3.66 (s, 6H), 2.15 (m, 1H), 1.42 (s, 9H), 1.38 (s, 6H), 0.93 (dd, 6H, J=1.7 and 6.7 Hz). LCMS: ret. time: 14.87 min.; purity: 93%; MS (m/e): 670 (MH⁺).

### 13. Synthesis of N4-(2,2-Dimethyl-3-oxo-4H-5-pyrido[1,4]oxazin-6-yl)-5-fluoro-N2-[[2-(morpholin-4-yl)ethoxy]carbonyl]-N2-(3,4,5-trimethoxyphenyl)-2,4-pyrimidinediamine

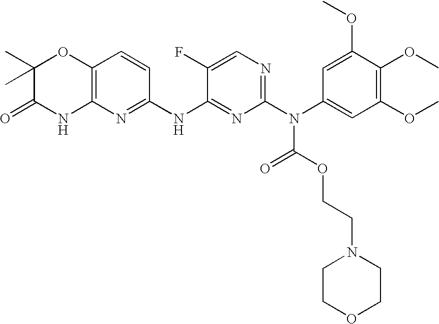

N4-(2,2-Dimethyl-3-oxo-4H-5-pyrido[1,4]oxazin-6-yl)-5-fluoro-N2-[[2-(morpholin-4-yl)ethoxy]carbonyl]-N2-(3,4,5-trimethoxyphenyl)-2,4-pyrimidinediamine was prepared from 4-(2-hydroxyethyl)morpholine and N2-chlorocarbonyl-N4-(2,2-dimethyl-3-oxo-4H-5-pyrido[1,4]oxazin-6-yl)-5-fluoro-N2-(3,4,5-trimethoxyphenyl)-2,4-pyrimidinediamine. The crude solid, obtained after concentration of the reaction mixture followed by treatment with aq. NaHCO₃, was purified by NEt₃ treated silica gel column chromatography. ¹H NMR (CDCl₃): δ 10.32 (s, 2H), 8.89 (s, 1H), 8.18 (d, 1H, J=2.9 Hz), 7.49 (d, 1H, J=8.8 Hz), 7.06 (d, 1H, J=8.8 Hz), 6.52 (s, 2H), 4.29 (m, 2H), 3.81 (s, 3H), 3.74 (s, 6H), 3.57 (m, 4H), 2.56 (m, 2H), 2.33 (m, 4H), 1.48 (s, 6H). LCMS: ret. time: 8.30 min.; purity: 92%; MS (m/e): 628 (MH⁺).

### 14. Synthesis of N2-[[2-(Carboxymethyl)amino]carbonyl]-N4-(2,2-dimethyl-3-oxo-4H-5-pyrido[1,4]oxazin-6-yl)-5-fluoro-N2-(3,4,5-trimethoxyphenyl)-2,4-pyrimidinediamine

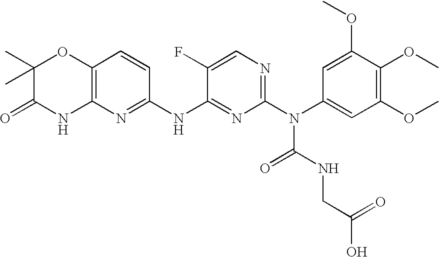

N2-[[2-(Carboxymethyl)amino]carbonyl]-N4-(2,2-dimethyl-3-oxo-4H-5-pyrido[1,4]oxazin-6-yl)-5-fluoro-N2-(3,4,5-trimethoxyphenyl)-2,4-pyrimidinediamine was prepared in the similar as described in the general procedure from glycine and N2-chlorocarbonyl-N4-(2,2-dimethyl-3-oxo-4H-5-pyrido[1,4]oxazin-6-yl)-5-fluoro-N2-(3,4,5-trimethoxyphenyl)-2,4-pyrimidinediamine. The crude concentrated reaction mixture was treated with 1N aq. HCl. The solid precipitated was dried and purified by preparative HPLC. ¹H NMR (DMSO-d6): δ 10.99 (s, 1H), 10.06 (t, 1H, J=5.0 Hz), 8.26 (d, 1H, J=3.8 Hz), 6.78 (app s, 2H), 6.51 (s, 2H), 3.85 (d, 2H, J=5.0 Hz), 3.71 (s, 3H), 3.67 (s, 6H), 1.37 (s, 6H). LCMS: ret. time: 9.74 min.; purity: 97%; MS (m/e): 572 (MH⁺).

### 15. Synthesis of 4-(2,2-Dimethyl-3-oxo-4H-5-pyrido[1,4]oxazin-6-yl)-5-fluoro-N2-[[1-methyl-piperidin-2-yl)methoxy]carbonyl]-N2-(3,4,5-trimethoxyphenyl)-2,4-pyrimidinediamine

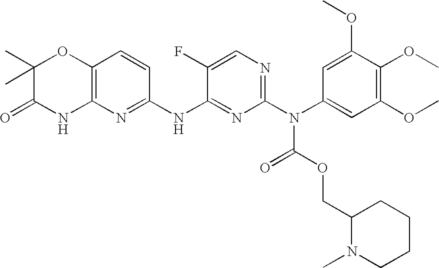

4-(2,2-Dimethyl-3-oxo-4H-5-pyrido[1,4]oxazin-6-yl)-5-fluoro-N2-[[1-methyl-piperidin-2-yl)methoxy]carbonyl]-N2-(3,4,5-trimethoxyphenyl)-2,4-pyrimidinediamine was prepared from N2-chlorocarbonyl-N4-(2,2-dimethyl-3-oxo-4H-5-pyrido[1,4]oxazin-6-yl)-5-fluoro-N2-(3,4,5-trimethoxyphenyl)-2,4-pyrimidinediamine and 1-methyl-2-piperidinemethanol in the similar manner as described in the general procedure. Crude off white solid obtained after the general workup was subjected to HPLC purification. ¹H NMR (DMSO-d6): δ 11.03 (s, 1H), 9.67 (s, 1H), 8.33 (d, 1H, J=3.0 Hz), 7.41 (d, 1H, J=8.5 Hz), 7.17 (d, 1H, J=8.5 Hz), 6.56 (s, 2H), 4.10 (d, 2H, J=4.7 Hz), 3.80 (s, 6H), 3.64 (s, 3H), 2.70-2.66 (m, 1H), 2.09 (s, 3H), 1.97-1.92 (m, 2H), 1.58-1.07 (m, 12H). LCMS: ret. time: 8.54 min.; purity: 92%; MS (m/e): 627 (MH⁺).

### 16. Synthesis of N2-[[[(2S)-2-(t-Butoxycarbonyl)amino-3-(1H-indol-3-yl)]propoxy]carbonyl]-N4-(2,2-dimethyl-3-oxo-4H-5-pyrido[1,4]oxazin-6-yl)-5-fluoro-N2-(3,4,5-trimethoxyphenyl)-2,4-pyrimidinediamine

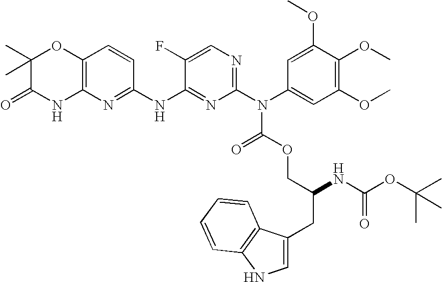

N2-[[[(2S)-2-(t-Butoxycarbonyl)amino-3-(1H-indol-3-yl)]propoxy]carbonyl]-N4-(2,2-dimethyl-3-oxo-4H-5-pyrido[1,4]oxazin-6-yl)-5-fluoro-N2-(3,4,5-trimethoxyphenyl)-2,4-pyrimidinediamine was prepared from N_{α}-(t-butoxycarbonyl)-L-tryptophanol and N2-chlorocarbonyl-N4-(2,2-dimethyl-3-oxo-4H-5-pyrido[1,4]oxazin-6-yl)-5-fluoro-N2-(3,4,5-trimethoxyphenyl)-2,4-pyrimidinediamine. The crude white solid collected after the workup was purified by NEt₃ treated silica gel column chromatography. ¹H NMR (DMSO-d6): δ 11.00 (s, 1H), 10.76 (s, 1H), 9.66 (s, 1H), 8.32 (d, 1H, J=3.2 Hz), 7.36 (d, 1H, J=8.8 Hz), 7.31-7.27 (m, 2H), 7.08 (d, 1H, J=8.5 Hz), 7.03-6.99 (m, 2H), 6.91-6.86 (m, 1H), 6.78 (d, 1H, J=8.2 Hz), 6.65 (s, 2H), 4.12-4.08 (m, 1H), 3.99-3.94 (m, 1H), 3.86-3.82 (m, 1H), 3.69 (s, 6H), 3.63 (s, 3H), 2.74 (m, 2H), 1.38 (s, 6H), 1.29 (s, 9H). LCMS: ret. time: 13.63 min.; purity: 91%; MS (m/e): 787 (MH⁺).

### 17. Synthesis of N2-[[[(2S)-2-Amino-3-(1H-indol-3-yl)]propoxy]carbonyl]-N4-(2,2-dimethyl-3-oxo-4H-5-pyrido[1,4]oxazin-6-yl)-5-fluoro-N2-(3,4,5-trimethoxyphenyl)-2,4-pyrimidinediamine

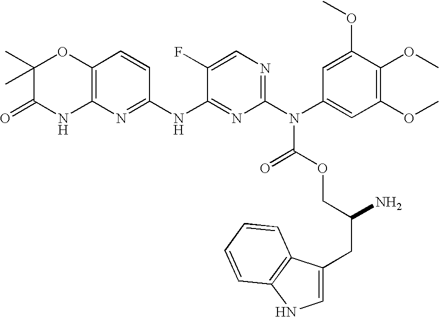

Trifluoracetic acid (0.04 mL, 59 mg, 0.519 mmol) was added to the stirring solution of N2-[[[(2S)-2-(t-butoxycarbonyl)amino-3-(1H-indol-3-yl)]propoxy]carbonyl]-N4-(2,2-dimethyl-3-oxo-4H-5-pyrido[1,4]oxazin-6-yl)-5-fluoro-N2-(3,4,5-trimethoxyphenyl)-2,4-pyrimidinediamine (93 mg, 0.118 mmol) in CH₂Cl₂ (5 mL) at 0° C. Progress of the reaction was monitored by LC/MS. Reaction mixture was concentrated after 1 hr of stirring the reaction mixture at 0° C. The crude was triturated with anhydrous Et₂O. Ethereal layer was decanted and dried to provide off white solid. The solid obtained was purified by HPLC to give 26 mg (32%) of N2-[[[(2S)-2-amino-3-(1H-indol-3-yl)]propoxy]carbonyl]-N4-(2,2-dimethyl-3-oxo-4H-5-pyrido[1,4]oxazin-6-yl)-5-fluoro-N2-(3,4,5-trimethoxyphenyl)-2,4-pyrimidinediamine as a white solid. LCMS: ret. time: 9.34 min.; purity: 92%; MS (m/e): 687 (MH⁺).

### 18. Synthesis of N4-(2,2-Dimethyl-3-oxo-4H-5-pyrido[1,4]oxazin-6-yl)-5-fluoro-N2-[[2-[4-(3-sulfopropyl)piperizin-1-yl]ethoxy]carbonyl]-N2-(3,4,5-trimethoxyphenyl)-2,4-pyrimidinediamine

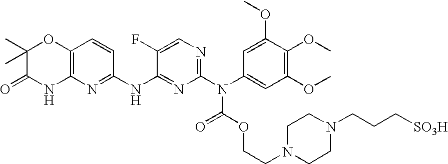

N4-(2,2-Dimethyl-3-oxo-4H-5-pyrido[1,4]oxazin-6-yl)-5-fluoro-N2-[[2-[4-(3-sulfopropyl)piperizin-1-yl]ethoxy]carbonyl]-N2-(3,4,5-trimethoxyphenyl)-2,4-pyrimidinediamine was prepared in the similar manner as described in the general procedure from N2-chlorocarbonyl-N4-(2,2-dimethyl-3-oxo-4H-5-pyrido[1,4]oxazin-6-yl)-5-fluoro-N2-(3,4,5-trimethoxyphenyl)-2,4-pyrimidinediamine and 4-(2-hydroxyethyl)-piperazinepropanesulfonic acid (EPPS) in CH₃CN. Reaction mixture was concentrated and diluted with water. The solid precipitated was filtered, dried and purified by preparative HPLC. ¹H NMR (DMSO-d6): δ 11.03 (s, 1H), 9.68 (s, 1H), 8.35 (d, 1H, J=3.2 Hz), 7.34 (d, 1H, J=8.8 Hz), 7.15 (d, 1H, J=8.8 Hz), 6.57 (s, 2H), 4.19 (m, 2H), 3.69 (s, 6H), 3.65 (s, 3H), 3.30-2.86 (m, 8H), 2.57-2.52 (m, 4H), 2.37-2.26 (m, 2H), 1.93-1.91 (m, 2H), 1.39 (s, 6H). LCMS: ret. time: 8.32 min.; purity: 98%; MS (m/e): 749 (MH⁺).

### 19. Synthesis of N2-[[2-(Dimethylamino)ethoxy]carbonyl]-N4-(2,2-dimethyl-3-oxo-4H-5-pyrido[1,4]oxazin-6-yl)-5-fluoro-N2-(3,4,5-trimethoxyphenyl)-2,4-pyrimidinediamine

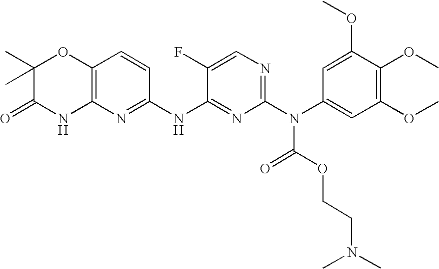

N2-[[2-(Dimethylamino)ethoxy]carbonyl]-N4-(2,2-dimethyl-3-oxo-4H-5-pyrido[1,4]oxazin-6-yl)-5-fluoro-N2-(3,4,5-trimethoxyphenyl)-2,4-pyrimidinediamine was prepared from N2-chlorocarbonyl-N4-(2,2-dimethyl-3-oxo-4H-5-pyrido[1,4]oxazin-6-yl)-5-fluoro-N2-(3,4,5-trimethoxyphenyl)-2,4-pyrimidinediamine and N,N-dimethylethanolamine. The crude solid obtained was purified by preparative HPLC. ¹H NMR (DMSO-d6): δ 11.04 (s, 1H), 9.68 (s, 1H), 8.33 (d, 1H, J=3.5 Hz), 7.39 (d, 1H, J=8.5 Hz), 7.16 (d, 1H, J=8.5 Hz), 6.54 (s, 2H), 4.17 (t, 2H, J=5.8 Hz), 3.68 (s, 6H), 3.64 (s, 3H), 2.45 (t, 2H, J=5.8 Hz), 2.08 (s, 6H), 1.39 (s, 6H). LCMS: ret. time: 8.87 min.; purity: 99%; MS (m/e): 586 (MH⁺).

### 20. Synthesis of (+/−)—N2-(1-Chloroethoxycarbonyl)-N4-(2,2-dimethyl-3-oxo-4H-5-pyrido[1,4]oxazin-6-yl)-5-fluoro-N2-(3,4,5-trimethoxyphenyl)-2,4-pyrimidinediamine

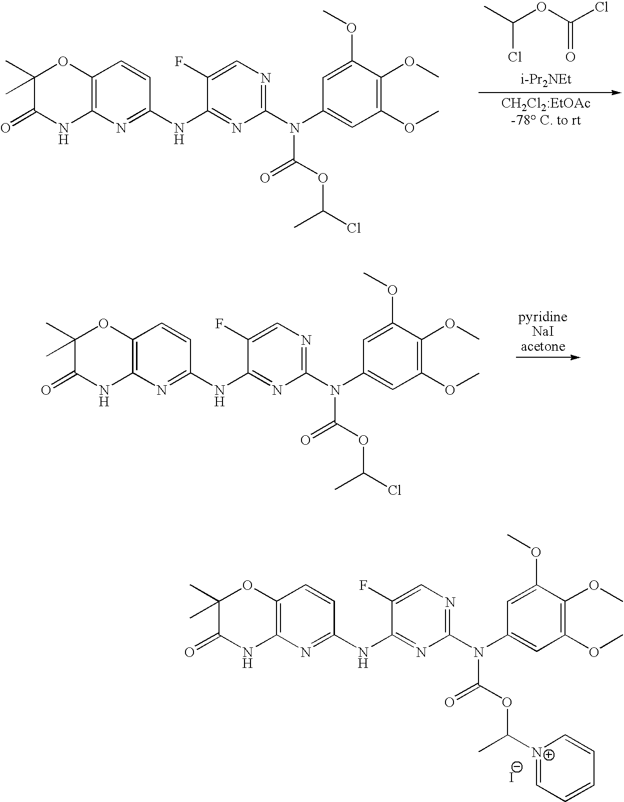

To pale yellow stirring mixture of N4-(2,2-dimethyl-3-oxo-4H-5-pyrido[1,4]oxazin-6-yl)-5-fluoro-N2-(3,4,5-trimethoxyphenyl)-2,4-pyrimidinediamine (250 mg, 0.53 mmol) and i-Pr₂NEt (0.14 mL, 102 mg, 0.78 mmol) in dicholoroethane (10 mL) at −78° C., 1-chloroethyl chloroformate (0.07 mL, 90 mg, 0.638 mmol) was added dropwise under nitrogen atmosphere for 5 min. The (clear, pale brown) reaction mixture was diluted with EtOAc (10 mL) at −78° C. after 1 h. Reaction mixture was allowed to warm to room temperature while stirring the contents by removing dry ice bath. Solid precipitated from pale brown transparent reaction mixture after stirring the contents at room temperature for 1 h. Reaction mixture was concentrated and diluted with water (15 mL). The solid precipitated was filtered and dried to provide (+/−)—N2-(1-chloroethoxycarbonyl)-N4-(2,2-dimethyl-3-oxo-4H-5-pyrido[1,4]oxazin-6-yl)-5-fluoro-N2-(3,4,5-trimethoxyphenyl)-2,4-pyrimidinediamine (250 mg, 81%) in 95% pure form. ¹H NMR (DMSO-d₆): δ 11.04 (s, 1H), 9.78 (s, 1H), 8.37 (d, 1H, J=3.2 Hz), 7.39 (d, 1H, J=8.5 Hz), 7.17 (d, 1H, J=8.5 Hz), 6.64 (qt, 1H, J=5.7 Hz), 6.57 (s, 2H), 3.69 (s, 6H), 3.65 (s, 3H), 1.65 (d, 3H, J=5.7 Hz), 1.39 (s, 6H). LCMS: ret. time: 10.35 min.; purity: 95%; MS (m/e): 578 (MH⁺).

### 21. Synthesis of (+/−)—N4-(2,2-Dimethyl-3-oxo-4H-5-pyrido[1,4]oxazin-6-yl)-5-fluoro-N2-[[1(1-pyridinium)ethoxy)carbonyl]]-N2-(3,4,5-trimethoxyphenyl)-2,4-pyrimidinediamine iodide salt

(+/−)—N2-(1-chloroethoxycarbonyl)-N4-(2,2-dimethyl-3-oxo-4H-5-pyrido[1,4]oxazin-6-yl)-5-fluoro-N2-(3,4,5-trimethoxyphenyl)-2,4-pyrimidinediamine (50 mg, 0.086 mmol) pyridine (34 mg, 0.43 mmol) and NaI (129 mg, 0.86 mmol) in acetone were stirred at room temperature for 24 h. Reaction mixture was concentrated, diluted with water (5 mL) and EtOAc (5 mL). The pale brown solid precipitated was filtered and dried to provide the desired material (+/−)—N-4-(2,2-dimethyl-3-oxo-4H-5-pyrido[1,4]oxazin-6-yl)-5-fluoro-N2-[[1(1-pyridinium)ethoxy)carbonyl]]-N2-(3,4,5-trimethoxyphenyl)-2,4-pyrimidinediamine iodide salt in 90% purity. The remaining impurity was characterized as N4-(2,2-dimethyl-3-oxo-4H-5-pyrido[1,4]oxazin-6-yl)-5-fluoro-N2-(3,4,5-trimethoxyphenyl)-2,4-pyrimidinediamine. LCMS: ret. time: 8.82 min.; purity: 90%; MS (m/e): 620 (M⁺).

### Example 2

### The Drug Compounds Inhibit RET Autophosphorylation

Materials

Control: Staurosporine 1 mM stock in DMSO (SIGMA, Cat# S4400)

Reagents:

Tyrosine Kinase Kit Green (Invitrogen, Cat# P2837)

Acetylated Bovine Gamma Globulin (BGG) (Invitrogen, Cat# P2255)

Active Ret Kinase (Upstate, Cat# 14-570)

Equipment: Fluorescence Polarization Plate Reader: Polarion, Tecan

Methods

Compounds were serially diluted in DMSO starting from 500× the desired final concentration and then diluted to 1% DMSO in kinase buffer (20 mM HEPES, pH 7.4, 5 mM MgCl₂, 2 mM MnCl₂, 1 mM DTT, 0.1 mg/mL acetylated BGG). Compound in 1% DMSO (0.2% DMSO final) was mixed with ATP in kinase buffer at room temperature.

The measurement of Ret autophosphorylation was initiated by the addition of kinase to the mixture of compound and ATP to give a final volume of 20 μL. Reactions were allowed to proceed at room temperature. The final reaction conditions and reaction time are summarized in Table 1.

TABLE 1
Final reaction conditions for the autophosphorylation of Ret

| | Enzyme Amount | ATP | Assay |
|---|---|---|---|
| Enzyme | per Reaction | Concentration | Time |
| Ret | 4 ng | 5 μM | 20 min |

The reactions were stopped by adding 20 μL of PTK quench mix containing EDTA/anti-phosphotyrosine antibody (1× final)/fluorescent phosphopeptide tracer (0.5× final) diluted in FP Dilution Buffer according to manufacturer's instructions (Invitrogen). The plates were incubated for 30 minutes in the dark at room temperature and then read on a Polarion fluorescence polarization plate reader (Tecan).

Data were converted to amount of phosphopeptide present using a calibration curve generated by competition with the phosphopeptide competitor provided in the Tyrosine Kinase Assay Kit, Green (Invitrogen). For IC₅₀ determination, the compound was tested at eleven concentrations in duplicate and curve-fitting was performed by non-linear regression analysis using Matlab version 6.5 (MathWorks, Inc., Natick, Mass., USA), yielding the values in Table 2 for inhibition of autophosphorylation.

TABLE 2

| Cmpd | | | IC₅₀ |
|---|---|---|---|
| No. | Structure | Name | (nM) |
| Control | | Staurosporine | 3.6 |
| 1 | 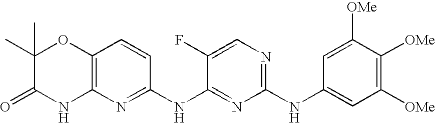 | 6-(5-fluoro-2-(3,4,5-trimethoxyphenyl-amino)pyrimidin-4-ylamino)-2,2-dimethyl-2H-pyrido[3,2-b][1,4]oxazin-3(4H)-one | 5.2 |
| 2 | 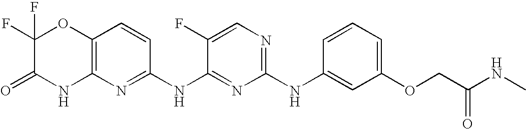 | 2-(3-(4-(2,2-difluoro-3-oxo-3,4-dihydro-2H-pyrido[3,2-b][1,4]oxazin-6-ylamino)-5-fluoropyrimidin-2-ylamino)phenoxy)-N-methylacetamide | 11.8 |
| 3 | 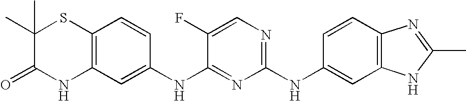 | 6-(5-fluoro-2-(2-methyl-1H-benzo[d]imidazol-6-ylamino)pyrimidin-4-ylamino)-2,3-dimethyl-2H-benzo[b][1,4]thiazin-3(4H)-one | 16.5 |
| 4 | 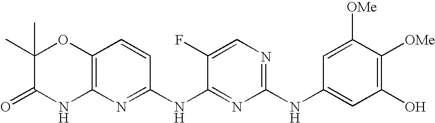 | 6-(5-fluoro-2-(3-hydroxy-4,5-dimethoxy-phenylamino)pyrimidin-4-ylamino)-2,2-dimethyl-2H-pyrido[3,2-b][1,4]oxazin-3(4H)-one | 7.3 |

### Example 3

### Acid Addition Salts

| Name | ¹H NMR |
|---|---|
| N4-(2,2-Dimethyl-3-oxo-4H-5- | 1H NMR (DMSO-d6): δ 11.31 (s, 1H), 9.89 (s, |
| pyrid[1,4]oxazin-6-yl)-5-fluoro-N2-(3,4,5- | 1H), 9.66 (s, 1H), 8.18 (d, J = 4.5 Hz, 1H), 7.55 |
| trimethoxyphenyl)-2,4-pyrimidinediamine | (d, J = 8.4 Hz, 1H), 7.30 (d, J = 8.7 Hz, 1H), 6.89 |
| Hydrogen Chloride Salt | (s, 2H), 3.65 (s, 6H), 3.61 (s, 3H), 1.43 (s, 6H). |
| N4-(2,2-Dimethyl-3-oxo-4H-5- | 1H NMR (DMSO-d6) δ 11.14 (s, 1H), 9.98 (s, |
| pyrid[1,4]oxazin-6-yl)-5-fluoro-N2-(3,4,5- | 1H), 9.63 (s, 1H), 8.17 (d, J = 3.9 Hz, 1H), 7.62- |
| trimethoxyphenyl)-2,4-pyrimidinediamine | 7.52 (m, 3H), 7.36-7.25 (m, 4H), 6.87 (s, 2H), |
| Benzenesulfonic Acid Salt | 3.66 (s, 6H), 3.61 (s, 3H), 1.43 (s, 6H). |
| N4-(2,2-Dimethyl-3-oxo-4H-5- | 1H NMR (DMSO-d6) δ 11.13 (s, 1H), 9.95 (s, |
| pyrid[1,4]oxazin-6-yl)-5-fluoro-N2-(3,4,5- | 1H), 9.62 (s, 1H), 8.18 (d, J = 3.9 Hz, 1H), 7.56 |
| trimethoxyphenyl)-2,4-pyrimidinediamine | (d, J = 9.0 Hz, 1H), 7.31 (d, J = 8.4 Hz, 1H), 6.88 |
| Methanesulfonic Acid Salt | (s, 2H), 3.66 (s, 6H), 3.61 (s, 3H), 2.33 (s, 3H), |
| | 1.43 (s, 6H). |
| N4-(2,2-Dimethyl-3-oxo-4H-5- | 1H NMR (DMSO-d6) δ 11.12 (s, 1H), 9.89 (s, |
| pyrid[1,4]oxazin-6-yl)-5-fluoro-N2-(3,4,5- | 1H), 9.57 (s, 1H), 8.17 (d, J = 3.9 Hz, 1H), 7.57 |
| trimethoxyphenyl)-2,4-pyrimidinediamine p- | (d, J = 8.4 Hz, 1H), 7.45 (d, J = 7.8 Hz, 2H), 7.31 |
| Toluene Sulfonic Acid Salt | (d, J = 8.4 Hz, 1H), 7.09 (d, J = 7.8 Hz, 2H), 6.89 |
| | (s, 2H), 3.66 (s, 6H), 3.61 (s, 3H), 2.28 (s, 3H), |
| | 1.43 (s, 6H). |
| N4-(2,2-Dimethyl-3-oxo-4H-5- | 1H NMR (DMSO-d6) δ 11.12 (s, 1H), 9.81 (s, |
| pyrid[1,4]oxazin-6-yl)-5-fluoro-N2-(3,4,5- | 1H), 9.53 (s, 1H), 8.16 (d, J = 4.2 Hz, 1H), 7.58 |
| trimethoxyphenyl)-2,4-pyrimidinediamine 4- | (d, J = 8.1 Hz, 2H), 7.37 (d, J = 8.1 Hz, 2H), 7.31 |
| Hydroxybenzenesulfonic Acid Salt | (d, J = 8.7 Hz, 1H), 6.90 (s, 2H), 6.64 (d, J = 8.7 |
| | Hz, 2H), 3.66 (s, 6H), 3.61 (s, 3H), 1.43 (s, 6H). |
| N4-(2,2-Dimethyl-3-oxo-4H-5- | 1H NMR (DMSO-d6) δ 11.10 (s, 1H), 9.72 (s, |
| pyrid[1,4]oxazin-6-yl)-5-fluoro-N2-(3,4,5- | 1H), 9.47 (s, 1H), 8.15 (d, J = 4.2 Hz, 1H), 7.62- |
| trimethoxyphenyl)-2,4-pyrimidinediamine | 7.56 (m, 1H), 7.31 (d, J = 8.1 Hz, 1H), 6.91 (s, |
| 2,4,6-Trimethylbenzenesulfonic Acid Salt | 2H), 6.72 (s, 2H), 3.66 (s, 6H), 3.61 (s, 3H), 2.48 |
| | (s, 6H), 2.16 (s, 3H), 1.43 (s, 6H). |
| N4-(2,2-Dimethyl-3-oxo-4H-5- | 1H NMR (DMSO-d6) δ 11.08 (s, 2H), 9.46 (s, |
| pyrid[1,4]oxazin-6-yl)-5-fluoro-N2-(3,4,5- | 2H), 9.30 (s, 2H), 8.91 (s, 1H), 8.70 (d, J = 5.4 |
| trimethoxyphenyl)-2,4-pyrimidinediamine 0.5 | Hz, 1H), 8.37 (dd, J = 1.5 and 7.8 Hz, 1H), 8.13 |
| Pyridine-3-sulfonic Acid Salt | (d, J = 3.6 Hz, 2H), 7.80-7.74 (m, 1H), 7.62 (d, J = |
| | 8.1 Hz, 2H), 7.31 (d, J = 8.1 Hz, 2H), 6.97 (s, |
| | 4H), 3.66 (s, 12H), 3.60 (s, 6H), 1.43 (s, 12H). |
| N4-(2,2-Dimethyl-3-oxo-4H-5- | 1H NMR (DMSO-d6) δ 11.08 (s, 1H), 9.44 (s, |
| pyrid[1,4]oxazin-6-yl)-5-fluoro-N2-(3,4,5- | 1H), 9.26 (s, 1H), 8.13 (d, J = 3.3 Hz, 1H), 7.63 |
| trimethoxyphenyl)-2,4-pyrimidinediamine p- | (d, J = 8.4 Hz, 1H), 7.47 (d, J = 8.1 Hz, 2H), 7.31 |
| Ethylbenzenesulfonic Acid Salt | (d, J = 8.1 Hz, 1H), 7.12 (d, J = 7.8 Hz, 2H), 6.97 |
| | (s, 2H), 3.65 (s, 6H), 3.59 (s, 3H), 2.57 (q, J = 7.8 |
| | Hz, 2H), 1.42 (s, 6H), 1.15 (t, J = 7.8 Hz, 3H). |
| N4-(2,2-Dimethyl-3-oxo-4H-5- | R932125 1H NMR (DMSO-d6) δ 11.08 (s, 2H), |
| pyrid[1,4]oxazin-6-yl)-5-fluoro-N2-(3,4,5- | 9.54 (s, 2H), 9.35 (s, 2H), 8.14 (d, J = 3.9 Hz, |
| trimethoxyphenyl)-2,4-pyrimidinediamine 0.5 | 2H), 7.60 (d, J = 8.4 Hz, 2H), 7.31 (d, J = 8.4 Hz, |
| 1,2-Ethanedisulfonic Acid Salt | 2H), 6.95 (s, 4H), 3.66 (s, 12H), 3.60 (s, 6H), |
| | 2.62 (s, 4H), 1.43 (s, 12H). |
| N4-(2,2-Dimethyl-3-oxo-4H-5- | 1H NMR (DMSO-d6) δ 11.11 (s, 1H), 9.83 (s, |
| pyrid[1,4]oxazin-6-yl)-5-fluoro-N2-(3,4,5- | 1H), 9.54 (s, 1H), 8.17 (d, J = 3.9 Hz, 1H), 7.57 |
| trimethoxyphenyl)-2,4-pyrimidinediamine | (d, J = 8.7 Hz, 1H), 7.30 (d, J = 8.4 Hz, 1H), 6.99 |
| (1R)-10-Camphorsulfonic Acid Salt | (s, 2H), 3.66 (s, 6H), 3.61 (s, 3H), 2.86 (d, J = |
| | 14.7 Hz, 1H), 2.67 (t, J = 9.9 Hz, 1H), 2.38 (d, J = |
| | 14.7 Hz, 1H), 2.22 (dt, J = 3.6 and 18.0 Hz, 1H), |
| | 1.93 (t, J = 4.5 Hz, 1H), 1.89-1.75 (m, 2H), 1.43 |
| | (s, 6H), 1.30-1.23 (m, 2H), 1.08 (t, J = 6.9 Hz, |
| | 1H), 1.04 (s, 3H), 0.74 (s, 3H). |
| N4-(2,2-Dimethyl-3-oxo-4H-5- | 1H NMR (DMSO-d6) δ 11.08 (s, 1H), 9.55 (s, |
| pyrid[1,4]oxazin-6-yl)-5-fluoro-N2-(3,4,5- | 1H), 9.36 (s, 1H), 8.14 (d, J = 3.9 Hz, 1H), 7.60 |
| trimethoxyphenyl)-2,4-pyrimidinediamine | (d, J = 8.7 Hz, 1H), 7.31 (d, J = 8.4 Hz, 1H), 6.94 |
| (1S)-10-Camphorsulfonic Acid Salt | (s, 2H), 3.66 (s, 6H), 3.60 (s, 3H), 2.85 (d, J = |
| | 14.7 Hz, 1H), 2.68 (t, 11.4 Hz, 1H), 2.36 (d, J = |
| | 14.7 Hz, 1H), 2.28-2.17 (m, 1H), 1.92 (t, J = 4.8 |
| | Hz, 1H), 1.89-1.74 (m, 2H), 1.43 (s, 6H), 1.26 (q, |
| | J = 10.8 Hz, 2H), 1.05 (s, 3H), 0.74 (s, 3H). |

### Example 4

### The Drug Compounds Inhibit RET Autophosphorylation

RET kinase is pre-diluted to a 10× working concentration prior to addition into the assay. The composition of the dilution buffer is 20 mM MOPS pH 7.0, 1 mM EDTA, 0.1% p-mercaptoethanol, 0.01% Brij-35, 5% glycerol and 1 mg/ml BSA.

In a final reaction volume of 25 μL, Ret (h) (5-10 mU) is incubated with 8 mM MOPS pH 7.0, 0.2 mM EDTA, 250 μL KKKSPCEYVNIEFC, 10 mM MgAcetate and [γ-³³P-ATP] specific activity approx. 500 cpm/μmol, concentration as required). The reaction is initiated by the addition of the MgATP mix. After incubation for 40 minutes at room temperature, the reaction is stopped by the addition of 5 μL of a 3% phosphoric acid solution. 10 μL of the reaction is then spotted onto a P30 filtermat and washed three times for 5 minutes in 75 mM phosphoric acid and once in methanol prior to drying and scintillation counting.

RET kinase autophosphorylation activity as a percentage of control RET kinase activity at various concentrations are reported in Table 3.

TABLE 3

| Cmpd | | | | | | | | | | |
|---|---|---|---|---|---|---|---|---|---|---|
| # | Structure | | | | | | | | | |
| 2 | 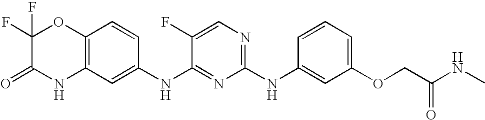 | | | | | | | | | |
| 5 | 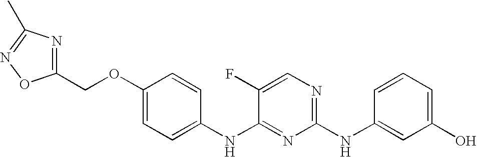 | | | | | | | | | |
| 6 | 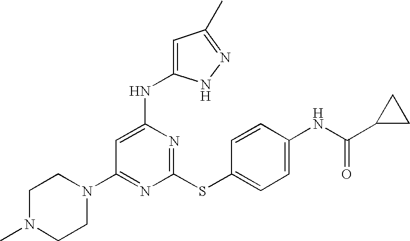 | | | | | | | | | |
| 7 | 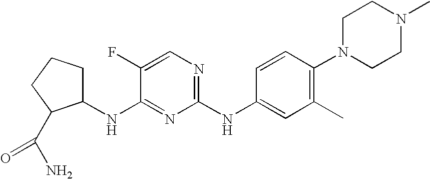 | | | | | | | | | |
| 8 | 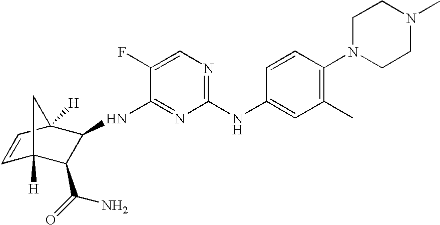 | | | | | | | | | |
| 9 | 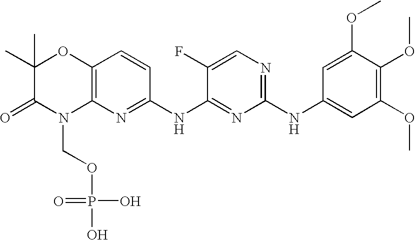 | | | | | | | | | |
| 10 | 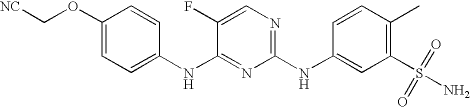 | | | | | | | | | |
| Cmpd | | Compound concentration, in micromoles | | | | | | | | |
| # | Name | 0.01 | 0.02 | 0.03 | 0.05 | 0.1 | 0.2 | 0.3 | 1 | 3 |
| 2 | 2-(3-(4-(2,2-difluoro-3-oxo-3,4-dihydro-2H-benzo[b][1,4]oxazin-6-ylamino)-5-fluoropyrimidin-2-ylamino)phenoxy-N-methyl-acetamide | | | 0 | | | | | 0 | 0 |
| 5 | 3-(5-fluoro-4-(4-((3-methyl-1,2,4-oxadiazol-5-yl)methoxy)phenylamino)pyrimidin-2-ylamino)phenol | 0 | | 0 | | 0 | | 0 | 0 | |
| 6 | N-(4-(4-(3-methyl-1H-pyrazol-5-ylamino)-6-(4-methylpiperazin-1-yl)pyrimidin-2-ylthio)phenyl)cyclopropanecarboxamide | | 78 | | | | 26 | | 4 | |
| 7 | 2-(5-fluoro-2-(3-methyl-4-(4-methylpiperazin-1-yl)phenylamino)pyrimidin-4-ylamino)cyclo-pentanecarboxamide | | 74 | | | | 34 | | 2 | |
| 8 | rac-3-(5-fluoro-2-(3-methyl-4-(4-methyl-piperazin-1-yl)phenylamino)pyrimidin-4-ylamino)bicyclo[2.2.1]hept-5-ene-2-carboxamide | 0 | | 0 | | 0 | | 0 | 0 | |
| 9 | (6-(5-fluoro-2-(3,4,5-trimethoxyphenylamino)pyrimidin-4-ylamino)-2,2-dimethyl-3-oxo-2H-pyrido[3,2-b][1,4]oxazin-4(3H)-yl)methyldihydrogenphosphate | | | | | | | 47 | 11 | |
| 10 | 5-(4-(4-cyanomethyl)phenylamino)-5-fluoro-pyrimidin-2-ylamino)-2-methylbenzene-sulfonamide | | | | | | 19 | | 1 | |

### Example 5

### The Drug Compounds Inhibit Proliferation of Tumor Cells without Cytotoxicity to Normal Cells

The 2,4-pyrimidinediamine drug and prodrug compounds of the methods herein disclosed were synthesized using methods described in U.S. application Ser. No. 11/337,049 filed Jan. 19, 2006 (US2006/0211657 A1), in particular, in the Examples section 7.1 at paragraphs 247-263 of the printed publication. Salts of the compounds were prepared using standard techniques, and used in screening tumor cell lines for antiproliferative activity.

In exemplary embodiments of the methods for inhibiting tumor cell proliferation using a besylate salt of 2,4-pyrimidinediamine drug Compound 1, the GI₅₀, TGI and LC₅₀ values of the drug were determined using standard in vitro antiproliferation assays. The effects of 2,4-pyrimidinediamine drug Compound 1 (supra, besylate salt) on tumor cell proliferation are illustrated in Table 4, below. A blank indicates that the drug compound was not tested against the specified cell line.

TABLE 4
Effect of drug on cancer cell proliferation

| Cancer | Panel/Cell | | | |
|---|---|---|---|---|
| Type | Line | GI₅₀ | TGI | LC₅₀ |
| Leukemia | CCRF-CEM | | >1.00E−4 | >1.00E−4 |
| | HL-60(TB) | >1.00E−4 | >1.00E−4 | >1.00E−4 |
| | K-562 | >1.00E−4 | >1.00E−4 | >1.00E−4 |
| | MOLT-4 | 4.82E−6 | >1.00E−4 | >1.00E−4 |
| | RPM1-8226 | >1.00E−4 | >1.00E−4 | >1.00E−4 |
| | SR | 3.56E−6 | >1.00E−4 | >1.00E−4 |
| Non-Small | A549/ATCC | | >1.00E−4 | >1.00E−4 |
| Cell Lung | EKVX | 1.66E−6 | >1.00E−4 | >1.00E−4 |
| Cancer | HOP-62 | 5.37E−7 | >1.00E−4 | >1.00E−4 |
| | HOP-92 | 4.10E−7 | | >1.00E−4 |
| | NCI-H226 | 4.97E−7 | | >1.00E−4 |
| | NCI-H23 | | >1.00E−4 | >1.00E−4 |
| | NCI-H322M | >1.00E−4 | >1.00E−4 | >1.00E−4 |
| | NCI-H460 | | >1.00E−4 | >1.00E−4 |
| | NCI-H522 | | >1.00E−4 | >1.00E−4 |
| Colon | COLO 205 | 7.26E−6 | >1.00E−4 | >1.00E−4 |
| Cancer | HCC-2998 | >1.00E−4 | >1.00E−4 | >1.00E−4 |
| | HCT-116 | | >1.00E−4 | >1.00E−4 |
| | HCT-15 | | >1.00E−4 | >1.00E−4 |
| | HT29 | 2.19E−6 | >1.00E−4 | >1.00E−4 |
| | KM12 | 3.55E−7 | >1.00E−4 | >1.00E−4 |
| | SW-620 | | >1.00E−4 | >1.00E−4 |
| CNS | SF-268 | 1.70E−6 | >1.00E−4 | >1.00E−4 |
| Cancer | SF-295 | | >1.00E−4 | >1.00E−4 |
| | SF-539 | 6.05E−7 | | >1.00E−4 |
| | SNB-19 | >1.00E−4 | >1.00E−4 | >1.00E−4 |
| | SNB-75 | 8.71E−7 | >1.00E−4 | >1.00E−4 |
| | U251 | | >1.00E−4 | >1.00E−4 |
| Melanoma | LOX I MVI | 1.13E−6 | 1.13E−6 | >1.00E−4 |
| | MALME-3M | >1.00E−4 | >1.00E−4 | >1.00E−4 |
| | M14 | >1.00E−4 | >1.00E−4 | >1.00E−4 |
| | SK-MEL-2 | >1.00E−4 | >1.00E−4 | >1.00E−4 |
| | SK-MEL-28 | >1.00E−4 | >1.00E−4 | >1.00E−4 |
| | SK-MEL-5 | >1.00E−4 | >1.00E−4 | >1.00E−4 |
| | UACC-257 | >1.00E−4 | >1.00E−4 | >1.00E−4 |
| | UACC-62 | | >1.00E−4 | >1.00E−4 |
| Ovarian | IGROV1 | 7.96E−7 | >1.00E−4 | >1.00E−4 |
| Cancer | OVCAR-3 | >1.00E−4 | >1.00E−4 | >1.00E−4 |
| | OVCAR-4 | >1.00E−4 | >1.00E−4 | >1.00E−4 |
| | OVCAR-5 | >1.00E−4 | >1.00E−4 | >1.00E−4 |
| | OVCAR-8 | >1.00E−4 | >1.00E−4 | >1.00E−4 |
| | SK-OV-3 | 4.29E−7, | >1.00E−4 | >1.00E−4 |
| Renal | 786-0 | 8.73E−7 | >1.00E−4 | >1.00E−4 |
| Cancer | A498 | 3.60E−7 | | >1.00E−4 |
| | ACHN | 1.24E−6 | >1.00E−4 | >1.00E−4 |
| | CAKI-1 | 5.15E−8 | 2.44E−6 | >1.00E−4 |
| | RXF 393 | 1.96E−7 | 7.74E−7 | >1.00E−4 |
| | SN12C | 6.98E−7 | >1.00E−4 | >1.00E−4 |
| | TK-10 | | >1.00E−4 | >1.00E−4 |
| | UO-31 | 5.96E−7 | >1.00E−4 | >1.00E−4 |
| | PC-3 | 3.07E−6 | >1.00E−4 | >1.00E−4 |
| Prostate | PC-3 | 3.07E−6 | >1.00E−4 | >1.00E−4 |
| Cancer | DU-145 | | >1.00E−4 | >1.00E−4 |
| Breast | MCF7 | 2.72E−6 | >1.00E−4 | >1.00E−4 |
| Cancer | NCI/ADR-RES | >1.00E−4 | >1.00E−4 | >1.00E−4 |
| | MDA-MB- | | >1.00E−4 | >1.00E−4 |
| | 231/ATCC | | | |
| | HS 578T | 1.19E−6 | >1.00E−4 | >1.00E−4 |
| | MDA-MB-435 | 3.62E−6 | >1.00E−4 | >1.00E−4 |
| | BT-549 | 2.43E−6 | >1.00E−4 | >1.00E−4 |
| | T-47D | | >1.00E−4 | >1.00E−4 |

As shown in Table 1, antiproliferative effects of the 2,4-pyrimidinediamine drug Compound 1 (besylate salt) were observed in several cell lines. As shown in the column labeled LC₅₀, these antiproliferative effects were apparently not attributable to cytotoxicity and/or cell death.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

All patents, patent applications, publications, and references cited herein are expressly incorporated by reference to the same extent as if each individual publication or patent application was specifically and individually indicated to be incorporated by reference.

What is claimed is:

1. A method for treating a disease or condition caused by a mutation in RET kinase, comprising administering to a subject in need of such treatment an amount of a compound according to the formula, 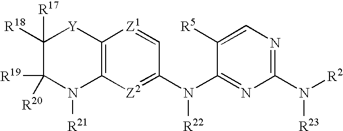
or a pharmaceutically acceptable salt, solvate, hydrate or N-oxide thereof, wherein:
Y is CH₂, NR²⁴, O, S, S(O), or S(O)₂;
Z¹ and Z² are each independently CH or N;
R² is lower alkyl optionally substituted with one or more of the same or different R⁸ groups, lower cycloalkyl optionally substituted with one or more of the same or different R⁸ groups, cyclohexyl optionally substituted with one or more of the same or different R⁸ groups, 3-8 membered heterocycloalkyl optionally substituted with one or more of the same or different R⁸ groups, (C₆-C₁₄) aryl optionally substituted with one or more of the same or different R⁸ groups, phenyl optionally substituted with one or more of the same or different R⁸ groups, or 5-15 membered heteroaryl optionally substituted with one or more of the same or different R⁸ groups;
R⁵ is halo, cyano, nitro, trihalomethyl or trifluoromethyl;
R⁸ is R^{a}, R^{b}, —B(OR^{a})₂, —B(R^{c}R^{c})₂, —(CH₂)ₘ—R^{b}, (CHR^{a})ₘ—R^{b}, —O—(CH₂)ₘ—R^{b}, S—(CH₂)ₘ—R^{b}, —O—CHR^{a}R^{b}, —O—CR^{a}(R^{b})₂, O—(CHR^{a})ₘ—R^{b}, —O(CH₂)ₘ—CH[(CH₂)ₘR^{b}]R^{b}, —S—(CHR^{a})ₘ—R^{b}, —O—C(O)NH—(CH₂)ₘ—R^{b}, —C(O)NH—(CHR^{a})ₘ —R^{b}, —O—(CH₂)ₘ—C(O)NH—(CH₂)ₘ—R^{b}, —S—(CH₂)ₘ—C(O)NH—(CH₂)ₘ—R^{b}, —O—(CHR^{a})ₘ—C(O)NH—(CHR^{a})R^{b}, —S—(CHR^{a})ₘ—C(O)NH—(CHR^{a})ₘ—R^{b}, —NH—(CH₂)ₘ—R^{k}, —NH—(CHR^{a})ₘ—R^{b}, —NH—[(CH₂)ₘR^{b}], —NH[(CH₂)ₘR^{b}]₂, NH—C(O)—NH—(CH₂)ₘ—R^{b}, —NH—C(O)—(CH₂)ₘ—CHR^{b}R^{b}, —NH—(CH₂)ₘ—C(O)—NH(CH₂)ₘ—R^{b}, R^{a} substituted with one to four, of the same or different R^{a} or R^{b}; or —OR^{a} substituted with one or more of the same or different R^{a} or R^{b};
R¹⁷ and R¹⁸ are independently hydrogen, halogen, fluoro, lower alkyl, or methyl;
or R¹⁷ and R¹⁸ taken together form an oxo (═O) group or, together with the carbon atom to which they are attached, form a spirocycle containing from 3 to 7 carbon atoms;
R¹⁹ and R²⁰ are independently hydrogen, lower alkyl, or methyl;
or R¹⁹ and R²⁰ taken together form an oxo (═O) group or, together with the carbon atom to which they are attached, form a spirocycle containing from 3 to 7 carbon atoms;
each R^{a} is independently hydrogen, lower alkyl, lower cycloalkyl, cyclohexyl, (C₄-C₁₁) cycloalkylalkyl, (C₆-C₁₀) aryl, phenyl, (C₇-C₁₆) arylalkyl, benzyl, 2-6 membered heteroalkyl, 3-8 membered heterocycloalkyl, morpholinyl, piperazinyl, homopiperazinyl, piperidinyl, 4-11 membered heterocycloalkylalkyl, 5-10 membered heteroaryl, or 6-16 membered heteroarylalkyl;
each R^{b} is independently ═O, —OR^{a}, (C₁-C₃) haloalkyloxy, ═S, —SR^{a}, ═NR^{a}, ═NOR^{a}, —NR^{c}R^{c}, halogen, —CF₃, —CN, —NC, —OCN, —SCN, —NO, —NO₂, ═N₂, —N₃, —S(O)R^{a}, —S(O)₂R^{a}, —S(O)₂OR^{a}, —S(O)NR^{c}R^{c}, —S(O)₂NR^{c}R^{c}, —OS(O)₂R^{a}, —OS(O)₂OR^{a}, —OS(O)₂NR^{c}R^{c}, —C(O)R^{a}, —C(O)OR^{a}, —C(O)NR^{c}R^{c}, —C(NH)NR^{c}R^{c} —C(NR^{a})NR^{c}R^{c}, —C(NOH)R^{a}, —C(NOH)NR^{c}R^{c}, —OC(O)R^{a}, —OC(O)OR^{a}, —OC(O)NR^{c}R^{c}, —OC(NH)NR^{c}R^{c}, —OC(NR^{a})NR^{c}R^{c}, —[NHC(O)]ₙR^{a}, —[NR^{a}C(O)]ₙR^{a}, —[NHC(O)]ₙOR^{a}, —[NR^{a}C(O)]ₙOR^{a}, —[NHC(O)]ₙNR^{c}R^{c}, —[NR^{a}C(O)]ₙR^{c}R^{c}, —[NHC(NH)]ₙNR^{c}R^{c}, —[NR^{a}C(NR^{a})]ₙNR^{c}R^{c};
each R^{c} is independently R^{a},
or two R^{c} bonded to the same nitrogen atom taken together with the nitrogen atom to which they are both attached form a 5 to 8-membered heterocycloalkyl or heteroaryl group comprising one or more of the same or different additional heteroatoms and optionally substituted with one to four of the same or different R^{a} groups;
R²¹, R²² and R²³ are each independently hydrogen or R^{p};
R²⁴ is hydrogen, lower alkyl, or R^{p};
each m is 1, 2, or 3; and
each n is 0, 1, 2, or 3,
with the proviso that at least one of R²¹, R²², R²³ and R²⁴ is R^{p}, wherein
each R^{p} is independently R^{p1} or R^{p2}, wherein
R^{p1} is —C(═X²)—X¹ —(CR⁵⁵R⁶⁵)_{q}—R⁷⁵, wherein
X¹ is O, S, or NR¹¹, wherein each R¹¹ is independently H or lower alkyl;
X² is O or S;
R⁵⁵ and R⁶⁵ are each independently H, OH, —OR¹¹, NR¹⁵R¹⁵, halo, lower alkyl, —C(O)O-alkyl, —C(O)OH, —OP(═O)(OR¹¹)₂, —OC(═O)OR¹¹, —OC(═O)R¹¹, cycloalkyl, aryl, heteroaryl or together form an oxo, wherein
each R¹⁵ is independently selected from H, lower alkyl, prenyl, allyl, —C(O)O-alkyl, cycloalkyl, aryl, heteroaryl, alkaryl and alkheteroaryl,
or two of R¹⁵ combine to form an optionally substituted heterocycloalkyl wherein each optionally substituted group is independently selected from R^{b};
R⁷⁵ is straight or branched, saturated or unsaturated alkyl, allyl, cycloalkyl, cycloalkyl, heterocycloalkyl, aryl, heteroaryl, prenylalkaryl, or heteroarylalkyl, each of which is optionally substituted wherein each optionally substituted with one or more R^{b} groups; and
q is an integer from 0 to 10; and
R^{p2} is —C(R^{d}R^{d})_{y}-A-R³, wherein
each R^{d} is independently hydrogen, cyano, —C(O)R^{e1}, —C(O)OR^{e1}, —C(O)NR^{e1}R^{e1}, —C(OR^{e1})(OR^{e1}), optionally substituted (C₁-C₂₀) alkyl, (C₁-C₂₀) perfluoroalkyl, optionally substituted (C₇-C₃₀) arylalkyl, or optionally substituted 6-30 membered heteroarylalkyl, wherein
each R^{e1} is independently hydrogen, alkyl, aryl, arylalkyl, heteroaryl, or heteroarylalkyl;
y is 1, 2, or 3;
A is O, S or NR⁵⁰, wherein R⁵⁰ is R^{d} or cycloalkyl; and
R³ is —R^{f}, —C(O)R^{f}, —C(O)O—R^{f}, —C(O)NR^{f}R^{f}, —Si(R^{f})₃, —P(O)(OH)₂, —P(O)(OH)(OR^{e}), —P(O)(OR^{e})₂, —P(OH)₂, —P(OH)(OR^{e}), or —P(OR^{e})₂, wherein
each R^{e} is independently (i) substituted or unsubstituted lower alkyl, substituted or unsubstituted (C₆-C₁₄) aryl, or substituted or unsubstituted (C₇-C₂₀) arylalkyl wherein each is optionally substituted with one or more groups independently selected from R^{b}, or (ii)-(CR^{d}R^{d})_{y}—OR^{f}, —(CR^{d}R^{d})_{y}—O—C(O)R^{f}, —(CR^{d}R^{d})_{y}—O—C(O)OR^{f}, —(CR^{d}R^{d})_{y}—S—C(O)R^{f}, —(CR^{d}R^{d})_{y}—S—C(O)OR^{f}, —(CR^{d}R^{d})_{y}—NH—C(O)R^{f}, —(CR^{d}R^{d})_{y}—NH—C(O)OR^{f}, or —Si(R^{f})₃;
or two R^{e} taken together with the oxygen atoms to which they are attached, form a 5-8 membered heterocycloalkyl group optionally substituted with substituted or unsubstituted lower alkyl, substituted or unsubstituted lower heterocycloalkyl, substituted or unsubstituted (C₆-C₁₄) aryl, substituted or unsubstituted (C₇-C₂₀) arylalkyl, or substituted or unsubstituted 5-14 membered heteroaryl wherein each is optionally substituted with one or more groups independently selected from R^{b};
each R^{f} group is independently hydrogen, optionally substituted lower alkyl, optionally substituted lower heteroalkyl, optionally substituted lower cycloalkyl, optionally substituted lower heterocycloalkyl, optionally substituted (C₆-C₁₀) aryl, optionally substituted 5-10 membered heteroaryl, optionally substituted (C₇-C₁₈) arylalkyl, or optionally substituted 6-18 membered heteroarylalkyl, wherein each is optionally substituted with one or more groups independently selected from R^{b},
or R⁵⁰ and R³ taken together with nitrogen atom to which they are both attached, form a three- to seven-membered ring;
effective to treat the cell proliferative disorder.

2. The method of claim 1, wherein the prodrug of a RET kinase inhibitory compound is of the formula,

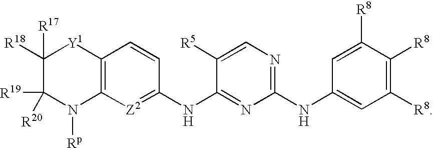

3. The method of claim 2, wherein R⁵ is fluoro.

4. The method of claim 2, wherein each R⁸ is independently hydrogen, hydroxy, or lower alkoxy.

5. The method of claim 2, wherein
R^{p} is —(CR^{d}R^{d})_{y}—P(O)(OH)(OH), —(CR^{d}R^{d})_{y}—O—P(O)(OH)(OR^{e}), —(CR^{d}R^{d})_{y}—O—P(O)(OR^{e})₂, —(CR^{d}R^{d})_{y}—O—P(OH)(OR^{e}), or —(CR^{d}R^{d})_{y}—O—P(OR^{e})₂, wherein
y is 1, 2, or 3;
each R^{e} is independently substituted or unsubstituted lower alkyl, substituted or unsubstituted (C₆-C₁₄) aryl, substituted or unsubstituted (C₇-C₂₀) arylalkyl, —(CR^{d}R^{d})_{y}—OR^{f}, —(CR^{d}R^{d})_{y}—O—C(O)R^{f}, —(CR^{d}R^{d})_{y}—O—C(O)OR¹, —(CR^{d}R^{d})_{y}—S—C(O)R^{f}, —(CR^{d}R^{d})_{y}—S—C(O)OR^{f}, —(CR^{d}R^{d})_{y}—NH—C(O)R^{f}, —(CR^{d}R^{d})_{y}—NH—C(O)OR^{e}, or —Si(R^{f})₃, wherein each R^{f} is independently hydrogen, substituted or unsubstituted lower alkyl, substituted or unsubstituted (C₆-C₁₄) aryl, or substituted or unsubstituted (C₇-C₂₀) arylalkyl.

6. The method of claim 5, wherein
R^{p} is —(CH₂)_{y}—O—P(O)(OH)(OH), —(CH₂)_{y}—O—P(O)(OH)(OR^{e}), —(CH₂)_{y}—O—P(O)(OR^{e})₂, —(CH₂)_{y}—O—P(OH)(OR^{e}), or —(CH₂)_{y}—O—P(OR^{e})₂, wherein
y is 1, 2, or 3;
each R^{e} is independently substituted or unsubstituted lower alkyl, substituted or unsubstituted (C₆-C₁₄) aryl, substituted or unsubstituted (C₇-C₂₀) arylalkyl.

7. The method of claim 6, wherein R^{p} is —CH₂—O—P(O)(OH)(OH).

8. The method of claim 1, wherein the prodrug of a RET kinase inhibitory compound is of the formula, 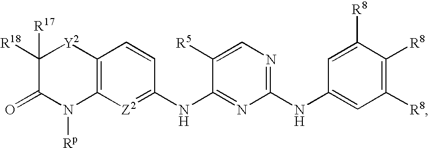
wherein Y² is O, S, S(O), or S(O)₂.

9. The method of claim 8, wherein R⁵ is fluoro.

10. The method of claim 8, wherein each R⁸ is independently hydrogen, hydroxy, or lower alkoxy.

11. The method of claim 8, wherein
R^{p} is —(CR^{d}R^{d})_{y}—O—P(O)(OH)(OH), —(CR^{d}R^{d})_{y}—O—P(O)(OH)(OR^{e}), —(CR^{d}R^{d})_{y}—O—P(O)(OR^{e})₂, —(CR^{d}R^{d})_{y}—O—P(OH)(OR^{e}), or —(CR^{d}R^{d})_{y}—O—P(OR^{e})₂, wherein
y is 1, 2, or 3;
each R^{e} is independently substituted or unsubstituted lower alkyl, substituted or unsubstituted (C₆-C₁₄) aryl, substituted or unsubstituted (C₇-C₂₀) arylalkyl, —(CR^{d}R^{d})_{y}—OR^{f}, —(CR^{d}R^{d})_{y}—O—C(O)R^{f}, —(CR^{d}R^{d})_{y}—O—C(O)OR^{f}, —(CR^{d}R^{d})_{y}—S—C(O)R^{f}, —(CR^{d}R^{d})_{y}—S—C(O)OR^{f}, —(CR^{d}R^{d})_{y}—NH—C(O)R^{f}, —(CR^{d}R^{d})_{y}—NH—C(O)OR^{f}, or —Si(R^{f})₃, wherein each R^{f} is independently hydrogen, substituted or unsubstituted lower alkyl, substituted or unsubstituted (C₆-C₁₄) aryl, or substituted or unsubstituted (C₇-C₂₀) arylalkyl.

12. The method of claim 11, wherein
R^{p} is —(CH₂)_{y}—O—P(O)(OH)(OH), —(CH₂)_{y}—O—P(O)(OH)(OR^{e}), —(CH₂)_{y}—O—P(O)(OR^{e})₂, —(CH₂)_{y}—O—P(OH)(OR^{e}), or —(CH₂)_{y}—O—P(OR^{e})₂, wherein
y is 1, 2, or 3;
each R^{e} is independently substituted or unsubstituted lower alkyl, substituted or unsubstituted (C₆-C₁₄) aryl, substituted or unsubstituted (C₇-C₂₀) arylalkyl.

13. The method of claim 12, wherein R^{p} is —CH₂—O—P(O)(OH)(OH).

14. The method of claim 1, wherein the compound is
6-(5-fluoro-2-(3,4,5-trimethoxyphenylamino)pyrimidin-4-ylamino)-2,2-dimethyl-2H-pyrido[3, 2-b][1,4]oxazin-3(4H)-one;
2-(3-(4-(2,2-difluoro-3-oxo-3,4-dihydro-2H-pyrido[3,2-b][1,4]oxazin-6-ylamino)-5-fluoropyrimidin-2-ylamino)phenoxy)-N-methylacetamide;
6-(5-fluoro-2-(2-methyl-1H-benzo[d]imidazol-6-ylamino)pyrimidin-4-ylamino)-2,2-dimethyl-2H-pyrido[3,2-b][1,4]thiazin-3(4H)-one;
6-(5-fluoro-2-(3-hydroxy-4,5-dimethoxyphenylamino)pyrimidin-4-ylamino)-2,2-dimethyl-2H-pyrido[3,2-b][1,4]oxazin-3(4H)-one;
or a pharmaceutically acceptable salt, solvate, hydrate, or N-oxide thereof.

15. The method of claim 1, wherein the disease or condition is a thyroid cancer.

16. The method of claim 1, wherein the disease or condition is medullary thyroid carcinoma, papillary thyroid carcinoma, multiple endocrine neoplasia type 2A (MEN2A), parathyroid adenoma, multiple endocrine neoplasia type 2B (MEN2B), familial medullary thyroid carcinoma (FMTC), pheochromocytoma or parathyroid hyperplasia.

17. The method claim 16, wherein the disease or condition is medullary thyroid carcinoma.

18. The method claim 1, in which the compound is administered in the form of a pharmaceutical composition.

19. The method claim 1, in which the compound is administered orally or intravenously.

20. A method of inhibiting proliferation of a thyroid tumor cell, comprising, administering to a tumor cell an amount of a prodrug of a RET kinase inhibitory compound effective to inhibit proliferation of the tumor cell of the formula: 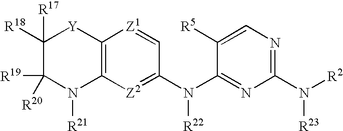
or a pharmaceutically acceptable salt, solvate, hydrate or N-oxide thereof, wherein:
Y is CH₂, NR²⁴, O, S, S(O), or S(O)₂;
Z¹ and Z² are each independently CH or N;
R² is lower alkyl optionally substituted with one or more of the same or different R⁸ groups, lower cycloalkyl optionally substituted with one or more of the same or different R⁸ groups, cyclohexyl optionally substituted with one or more of the same or different R⁸ groups, 3-8 membered heterocycloalkyl optionally substituted with one or more of the same or different R⁸ groups, (C₆-C₁₄) aryl optionally substituted with one or more of the same or different R⁸ groups, phenyl optionally substituted with one or more of the same or different R⁸ groups, or 5-15 membered heteroaryl optionally substituted with one or more of the same or different R⁸ groups;
R⁵ is halo, fluoro, cyano, nitro, trihalomethyl, or trifluoromethyl;
R⁸ is R^{a}, R^{b}, —B(OR^{a})₂, —B(R^{c}R^{c})₂, —(CH₂)ₘ—R^{b}, —(CHR^{a})ₘ—R^{b}, —O—(CH₂)ₘ—R^{b}, S—(CH₂)ₘ—R^{b}, —O—CHR^{a}R^{b}, —O—CR^{a}(R^{b})₂, O—(CHR^{a})ₘ—R^{b}, —O—(CH₂)ₘ—CH[(CH₂)ₘR^{b}]R^{b}, —O—(CHR^{a})ₘ—R^{b}, —O—C(O)NH—(CH₂)ₘ—R^{b}, —C(O)NH—(CHR^{a})ₘ —R^{b}, —O—(CH₂)ₘ—C(O)NH—(CH₂)ₘ—R^{b}, —S—(CH₂)ₘ—C(O)NH—(CH₂)ₘ—R^{b}, O—(CHR^{a})ₘ—C(O)NH—(CHR^{a})—R^{b}, —S(CHR^{a})ₘ—C(O)NH—(CHR^{a})ₘ—R^{b}, —NH—(CH₂)ₘ—R^{b}, —NH—(CHR^{a})ₘ—R^{b}, —NH—(CH₂)ₘR^{b}, —NH[(CH₂)ₘR^{b}]₂, NH—C(O)—NH—(CH₂)ₘ—R^{b}, —NH—C(O)—(CH₂)ₘ—CHR^{b}R^{b}, —NH—(CH₂)ₘ—R^{b}, R^{a} substituted with one to four, of the same or different R^{a} or R^{b}; or —OR^{a} substituted with one or more of the same or different R^{a} or R^{b};
R¹⁷ and R¹⁸ are independently hydrogen, halogen, fluoro, lower alkyl, or methyl;
or R¹⁷ and R¹⁸ taken together form an oxo (═O) group or, together with the carbon atom to which they are attached, form a spirocycle containing from 3 to 7 carbon atoms;
R¹⁹ and R²⁰ are independently hydrogen, lower alkyl, or methyl;
or R¹⁹ and R²⁰ taken together form an oxo (═O) group or, together with the carbon atom to which they are attached, form a spirocycle containing from 3 to 7 carbon atoms;
each R^{a} is independently hydrogen, lower alkyl, lower cycloalkyl, cyclohexyl, (C₄-C₁₁) cycloalkylalkyl, (C₆-C₁₀) aryl, phenyl, (C₇-C₁₆) arylalkyl, benzyl, 2-6 membered heteroalkyl, 3-8 membered heterocycloalkyl, morpholinyl, piperazinyl, homopiperazinyl, piperidinyl, 4-11 membered heterocycloalkylalkyl, 5-10 membered heteroaryl, or 6-16 membered heteroarylalkyl;
each R^{b} is independently ═O, —OR^{a}, (C₁-C₃) haloalkyloxy, ═S, —SR^{a}, ═NR^{a}, ═NOR^{a}, —NR^{c}R^{c}, halogen, —CF₃, —CN, —NC, —OCN, —SCN, —NO, —NO₂, ═N₂, —N₃, —S(O)R^{a}, —S(O)₂R^{a}, —S(O)₂OR^{a}, —S(O)NR^{c}R^{c}, —S(O)₂NR^{c}R^{c}, —OS(O)₂R^{a}, —OS(O)₂OR^{a}, —OS(O)₂NR^{c}R^{c}, —C(O)R^{a}, —C(O)OR^{a}, —C(O)NR^{c}R^{c}, —C(NH)NR^{c}R^{c} —C(NR^{a})NR^{c}R^{c}, —C(NOH)R^{a}, —C(NOH)NR^{c}R^{c}, —OC(O)R^{a}, —OC(O)OR^{a}, —OC(O)NR^{c}R^{c}, —OC(NH)NR^{c}R^{c}, —OC(NR^{a})NR^{c}R^{c}, —[NHC(O)]ₙR^{a}, —[NR^{a}C(O)]ₙR^{a}, —[NHC(O)]ₙOR^{a}, —[NR^{a}C(O)]ₙOR^{a}, —[NHC(O)]ₙNR^{c}R^{c}, —[NR^{a}C(O)]ₙR^{c}R^{c}, —[NHC(NH)]ₙNR^{c}R^{c}, or —[NR^{a}C(NR^{a})]NR^{c}R^{c};
each R^{c} is independently R^{a},
or two R^{c} bonded to the same nitrogen atom are taken together, with the nitrogen atom to which they are both attached, form a 5 to 8-membered heterocycloalkyl or heteroaryl group comprising one or more of the same or different additional heteroatoms and optionally substituted with one to four of the same or different R^{a} groups;
R²¹, R²² and R²³ are each independently hydrogen or R^{p};
R²⁴ is hydrogen, lower alkyl, or R^{p};
each m is 1, 2, or 3; and
each n is 0, 1, 2, or 3;
with the proviso that at least one of R²¹, R²², R²³ and R²⁴ is a R^{p}, wherein
each R^{p} is independently R^{p1} or R^{p2}, wherein
R^{p1} is —C(═X²)—X¹—(CR⁵⁵R⁶⁵)_{q}—R⁷⁵, wherein
X¹ is O, S, or NR¹¹, wherein each R¹¹ is independently H or lower alkyl;
X² is O or S;
R⁵⁵ and R⁶⁵ are each independently H, OH, —OR¹¹, NR¹⁵R¹⁵, halo, lower alkyl, —C(O)O-alkyl, —C(O)OH, —OP(═O)(OR¹¹)₂, —OC(═O)OR¹¹, —OC(═O)R¹¹, cycloalkyl, aryl, heteroaryl or together form an oxo, wherein
each R¹⁵ is independently selected from H, lower alkyl, prenyl, allyl, —C(O)O-alkyl, cycloalkyl, aryl, heteroaryl, alkaryl and alkheteroaryl,
or two of R¹⁵ combine to form an optionally substituted heterocycloalkyl wherein each optionally substituted group is independently selected from R^{b};
R⁷⁵ is straight or branched, saturated or unsaturated alkyl, allyl, cycloalkyl, cycloalkyl, heterocycloalkyl, aryl, heteroaryl, prenylalkaryl, or heteroarylalkyl, each of which is optionally substituted wherein each optionally substituted with one or more R^{b} groups; and
q is an integer from 0 to 10; and
R^{p2} is —C(R^{d}R^{d})_{y}-A-R³, wherein
each R^{d} is independently hydrogen, cyano, —C(O)R^{e1}, —C(O)OR^{e1}, —C(O)NR^{e1}R^{e1}, —C(OR^{e1})(OR^{e1}), optionally substituted (C₁-C₂₀) alkyl, (C₁-C₂₀) perfluoroalkyl, optionally substituted (C₇-C₃₀) arylalkyl, or optionally substituted 6-30 membered heteroarylalkyl, wherein
each R^{e1} is independently hydrogen, alkyl, aryl, arylalkyl, heteroaryl, or heteroarylalkyl;
y is 1, 2, or 3;
A is O, S or NR⁵⁰, wherein R⁵⁰ is R^{d} or cycloalkyl; and
R³ is —R^{f}, —C(O)R^{f}, —C(O)O—R^{f}, —C(O)NR^{f}R^{f}, —Si(R^{f})₃, —P(O)(OH)₂, —P(O)(OH)(OR^{e}), —P(O)(OR^{e})₂, —P(OH)₂, —P(OH)(OR^{e}), or —P(OR^{e})₂, wherein
each R^{e} is independently (i) substituted or unsubstituted lower alkyl, substituted or unsubstituted (C₆-C₁₄) aryl, or substituted or unsubstituted (C₇-C₂₀) arylalkyl wherein each is optionally substituted with one or more groups independently selected from R^{b}, or (ii) —(CR^{d}R^{d})_{y}—OR^{f}, —(CR^{d}R^{d})_{y}—O—C(O)R^{f}, —(CR^{d}R^{d})_{y}—O—C(O)OR^{f}, —(CR^{d}R^{d})_{y}—S—C(O)R^{f}, —(CR^{d}R^{d})_{y}—S—C(O)OR^{f}, —(CR^{d}R^{d})_{y}—NH—C(O)R^{f}, —(CR^{d}R^{d})_{y}—NH—C(O)OR^{f}, or —Si(R^{f})₃;
or two R^{e} taken together with the oxygen atoms to which they are attached, form a 5-8 membered heterocycloalkyl group optionally substituted with substituted or unsubstituted lower alkyl, substituted or unsubstituted lower heterocycloalkyl, substituted or unsubstituted (C₆-C₁₄) aryl, substituted or unsubstituted (C₇-C₂₀) arylalkyl, or substituted or unsubstituted 5-14 membered heteroaryl wherein each is optionally substituted with one or more groups independently selected from R^{b};
each R^{f} group is independently hydrogen, optionally substituted lower alkyl, optionally substituted lower heteroalkyl, optionally substituted lower cycloalkyl, optionally substituted lower heterocycloalkyl, optionally substituted (C₆-C₁₀) aryl, optionally substituted 5-10 membered heteroaryl, optionally substituted (C₇-C₁₈) arylalkyl, or optionally substituted 6-18 membered heteroarylalkyl, wherein each is optionally substituted with one or more groups independently selected from R^{b},
or R⁵⁰ and R³ taken together with nitrogen atom to which they are both attached, form a three- to seven-membered ring.

21. The method of claim 20 wherein the prodrug of a RET kinase inhibitory compound is of the formula,

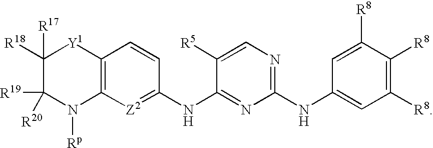

22. The method of claim 21, wherein R⁵ is fluoro.

23. The method of claim 21, wherein each R⁸ is independently hydrogen, hydroxy, or lower alkoxy.

24. The method of claim 21, wherein
R^{p} is —(CR^{d}R^{d})_{y}—O—P(O)(OH)(OH), —(CR^{d}R^{d})_{y}—O—P(O)(OH)(OR^{e}), —(CR^{d}R^{d})_{y}—O—P(O)(OR^{e})₂, —(CR^{d}R^{d})_{y}—O—P(OH)(OR^{e}), or —(CR^{d}R^{d})_{y}—O—P(OR^{e})₂, wherein
y is 1, 2, or 3;
each R^{e} is independently substituted or unsubstituted lower alkyl, substituted or unsubstituted (C₆-C₁₄) aryl, substituted or unsubstituted (C₇-C₂₀) arylalkyl, —(CR^{d}R^{d})_{y}—OR^{f}, —(CR^{d}R^{d})_{y}—OC(O)R^{f}, —(CR^{d}R^{d})_{y}—O—C(O)OR^{f}, —(CR^{d}R^{d}), —S—C(O)R^{f}, —(CR^{d}R^{d})_{y}—S—C(O)OR^{f}, —(CR^{d}R^{d})_{y}—NH—C(O)R^{f}, —(CR^{d}R^{d})_{y}—NH—C(O)OR^{f}, or —Si(R^{f})₃, wherein each R^{f} is independently hydrogen, substituted or unsubstituted lower alkyl, substituted or unsubstituted (C₆-C₁₄) aryl, or substituted or unsubstituted (C₇-C₂₀) arylalkyl.

25. The method of claim 24, wherein
R^{p} is —(CH₂)_{y}—O—P(O)(OH)(OH), —(CH₂)_{y}—O—P(O)(OH)(OR^{e}), —(CH₂)_{y}—O—P(O)(OR^{e})₂, —(CH₂)_{y}—O—P(OH)(OR^{e}), or —(CH₂)_{y}—O—P(OR^{e})₂, wherein
y is 1, 2, or 3;
each R^{e} is independently substituted or unsubstituted lower alkyl, substituted or unsubstituted (C₆-C₁₄) aryl, substituted or unsubstituted (C₇-C₂₀) arylalkyl.

26. The method of claim 25, wherein R^{p} is —CH₂—O—P(O)(OH)(OH).

27. The method of claim 20, wherein the prodrug of a RET kinase inhibitory compound is of the formula, 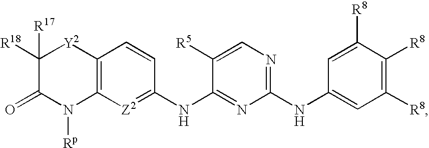
wherein Y² is O, S, S(O) or S(O)₂.

28. The method of claim 27, wherein R⁵ is fluoro.

29. The method of claim 27 wherein each R⁸ is independently hydrogen, hydroxy, or lower alkoxy.

30. The method of claim 27, wherein
R^{p} is —(CR^{d}R^{d})_{y}—O—P(O)(OH)(OH), —(CR^{d}R^{d})_{y}—O—P(O)(OH)(OR^{e}), —(CR^{d}R^{d})_{y}—O—P(O)(OR^{e})₂, —(CR^{d}R^{d})_{y}—O—P(OH)(OR^{e}), or —(CR^{d}R^{d})_{y}—O—P(OR^{e})₂, wherein
y is 1, 2, or 3;
each R^{e} is independently substituted or unsubstituted lower alkyl, substituted or unsubstituted (C₆-C₁₄) aryl, substituted or unsubstituted (C₇-C₂₀) arylalkyl, —(CR^{d}R^{d})_{y}—OR^{f}, —(CR^{d}R^{d})_{y}—O—C(O)R^{f}, —(CR^{d}R^{d})_{y}—O—C(O)OR^{f}, —(CR^{d}R^{d})_{y}—S—C(O)R^{f}, —(CR^{d}R^{d})_{y}—S—C(O)OR^{f}, —(CR^{d}R^{d})_{y}—NH—C(O)R^{f}, —(CR^{d}R^{d})_{y}—NH—C(O)OR^{f}, or —Si(R^{f})₃; each R^{f} is independently hydrogen, substituted or unsubstituted lower alkyl, substituted or unsubstituted (C₆-C₁₄) aryl, or substituted or unsubstituted (C₇-C₂₀) arylalkyl.

31. The method of claim 30, wherein
R^{f} is —(CH₂)_{y}—O—P(O)(OH)(OH), —(CH₂)_{y}—O—P(O)(OH)(OR^{e}), —(CH₂)_{y}—O—P(O)(OR^{e})₂, —(CH₂)_{y}—O—P(OH)(OR^{e}), or —(CH₂)_{y}—O—P(OR^{e})₂, wherein
y is 1, 2, or 3;
each R^{e} is independently substituted or unsubstituted lower alkyl, substituted or unsubstituted (C₆-C₁₄) aryl, substituted or unsubstituted (C₇-C₂₀) arylalkyl.

32. The method of claim 31 wherein R^{p} is —CH₂—O—P(O)(OH)(OH).

33. The method of claim 1, wherein the compound is
6-(5-fluoro-2-(3,4,5-trimethoxyphenylamino)pyrimidin-4-ylamino)-2,2-dimethyl-2H-pyrido[3, 2-b][1,4]oxazin-3(4H)-one;
2-(3-(4-(2,2-difluoro-3-oxo-3,4-dihydro-2H-pyrido[3,2-b][1,4]oxazin-6-ylamino)-5-fluoropyrimidin-2-ylamino)phenoxy)-N-methylacetamide;
6-(5-fluoro-2-(2-methyl-1H-benzo[d]imidazol-6-ylamino)pyrimidin-4-ylamino)-2,2-dimethyl-2H-pyrido[3,2-b][1,4]thiazin-3(4H)-one;
6-(5-fluoro-2-(3-hydroxy-4,5-dimethoxyphenylamino)pyrimidin-4-ylamino)-2,2-dimethyl-2H-pyrido[3,2-b][1,4]oxazin-3(4H)-one;
or a pharmaceutically acceptable salt, solvate, hydrate, or N-oxide thereof.

34. The method of claim 1, which is carried out in vitro.

35. The method of claim 1, which is carried out in vivo in a subject.

36. The method of claim 1, in which the tumor cell is a thyroid tumor cell.

37. The method of claim 35, in which the compound is administered in the form of a pharmaceutical composition.

38. The method of claim 35, in which the compound is administered orally or intravenously.

39. A method of treating a solid thyroid tumor cancer in a subject, comprising administering to a subject an amount of a compound effective to treat the solid tumor cancer according to the formula: 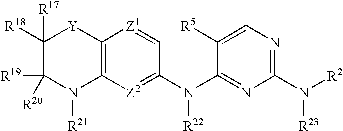
or a pharmaceutically acceptable salt, solvate, hydrate or N-oxide thereof, wherein:
Y is CH₂, NR²⁴, O, S, S(O), or S(O)₂;
Z¹ and Z² are each independently CH or N;
R² is lower alkyl optionally substituted with one or more of the same or different R⁸ groups, lower cycloalkyl optionally substituted with one or more of the same or different R⁸ groups, cyclohexyl optionally substituted with one or more of the same or different R⁸ groups, 3-8 membered heterocycloalkyl optionally substituted with one or more of the same or different R⁸ groups, (C₆-C₁₄) aryl optionally substituted with one or more of the same or different R⁸ groups, phenyl optionally substituted with one or more of the same or different R⁸ groups, or 5-15 membered heteroaryl optionally substituted with one or more of the same or different R⁸ groups;
R⁵ is halo, fluoro, cyano, nitro, trihalomethyl, or trifluoromethyl;
R⁸ is R^{a}, R^{b}, —B(OR^{a})₂, —B(R^{c}R^{c})₂, —(CH₂)ₘ—R^{b}, —(CHR^{a})ₘ—R^{b}, —O—(CH₂)ₘ—R^{b}, S—(CH₂)ₘ—R^{b}, —CHR^{a}R^{b}, —O—CR^{a}(R^{b})₂, —O—(CHR^{a})ₘ—R^{b}, —O—(CH₂)ₘ—CH[(CH₂)ₘR^{b}]R^{b}, —S—(CHR^{a})ₘ—R^{b}, —O—C(O)NH—(CH₂)ₘ—R^{b}, —C(O)NH—(CHR^{a})ₘ—R^{b}, —O—(CH₂)ₘ—C(O)NH—(CH₂)ₘ—R^{b}, —S—(CH₂)ₘ—C(O)NH—(CH₂)ₘ—R^{b}, O—(CHR^{a})ₘ—C(O)NH—(CHR^{a})_R^{b}, —S(CHR^{a})ₘ—C(O)NH—(CHR^{a})ₘ—R^{b}, —NH—(CH₂)ₘ—R^{b}, —NH—(CHR^{a})ₘ—R^{b}, —NH—[(CH₂)ₘR^{b}], NH[(CH₂)ₘR^{b}]₂, NH—C(O)—NH—(CH₂)ₘ—R^{b}, —NH—C(O)—(CH₂)ₘ—CHR^{b}R^{b}, —NH—(CH₂)ₘ—C(O)—NH—(CH₂)ₘ—; R^{a} substituted with one to four, of the same or different R^{a} or R^{b}; or —OR^{a} substituted with one or more of the same or different R^{a} or R^{b};
R¹⁷ and R¹⁸ are independently hydrogen, halogen, fluoro, lower alkyl or methyl;
or R¹⁷ and R¹⁸ taken together form an oxo (═O) group or, together with the carbon atom to which they are attached, form a spirocycle containing from 3 to 7 carbon atoms;
R¹⁹ and R²⁰ are independently hydrogen, lower alkyl, or methyl;
or R¹⁹ and R²⁰ taken together form an oxo (═O) group or, together with the carbon atom to which they are attached, form a spirocycle containing from 3 to 7 carbon atoms;
each R^{a} is independently hydrogen, lower alkyl, lower cycloalkyl, cyclohexyl, (C₄-C₁₁) cycloalkylalkyl, (C₆-C₁₀) aryl, phenyl, (C₇-C₁₆) arylalkyl, benzyl, 2-6 membered heteroalkyl, 3-8 membered heterocycloalkyl, morpholinyl, piperazinyl, homopiperazinyl, piperidinyl, 4-11 membered heterocycloalkylalkyl, 5-10 membered heteroaryl, or 6-16 membered heteroarylalkyl;
each R^{b} is independently ═O, —OR^{a}, (C₁-C₃) haloalkyloxy, ═S, —SR^{a}, ═NR^{a}, ═NOR^{a}, —NR^{c}R^{c}, halogen, —CF₃, —CN, —NC, —OCN, —SCN, —NO, —NO₂, ═N₂, —N₃, —S(O)R³, —S(O)₂R³, —S(O)₂OR^{a}, —S(O)NR^{c}R^{c}, —S(O)₂NR^{c}R^{c}, —OS(O)₂R^{a}, —OS(O)₂OR^{a}, —OS(O)₂NR^{c}R^{c}, —C(O)R^{a}, —C(O)OR^{a}, —C(O)NR^{c}R^{c}, —C(NH)NR^{c}R^{c} —C(NR^{a})NR^{c}R^{c}, —C(NOH)R^{a}, —C(NOH)NR^{c}R^{c}, —OC(O)R^{a}, —OC(O)OR^{a}, —OC(O)NR^{c}R^{c}, —OC(NH)NR^{c}R^{c}, —OC(NR^{a})NR^{c}R^{c}, —[NHC(O)]ₙR^{a}, —[NR^{a}C(O)]ₙR^{a}, —[NHC(O)]ₙOR^{a}, —[NR^{a}C(O)]ₙOR^{a}, —[NHC(O)]ₙNR^{c}R^{c}, —[NR^{a}C(O)]ₙNR^{c}R^{c}, —[NHC(NH)]ₙNR^{c}R^{c}, or —[NR^{a}C(NR^{a})]ₙNR^{c}R^{c};
each R^{e} is independently R^{a},
or two R^{e} bonded to the same nitrogen atom are taken together with that nitrogen atom to form a 5 to 8-membered heterocycloalkyl or heteroaryl group comprising one or more of the same or different additional heteroatoms and optionally substituted with one to four of the same or different R^{a} groups;
R²¹, R²² and R²³ are each independently hydrogen or R^{p};
R²⁴ is hydrogen, lower alkyl, or R^{p};
each m is independently 1, 2, or 3; and
each n is independently 0, 1, 2, or 3,
with the proviso that at least one of R²¹, R²², R²³ and R²⁴ is a R^{p}, wherein
each R^{p} is independently R^{p1} or R^{p2}, wherein
R^{p1} is —C(═X²)—X¹—(CR⁵⁵R⁶⁵)_{q}—R⁷⁵, wherein
X¹ is O, S, or NR¹¹, wherein each R¹¹ is independently H or lower alkyl;
X² is O or S;
R⁵⁵ and R⁶⁵ are each independently H, OH, —OR¹¹, NR¹⁵R¹⁵, halo, lower alkyl, —C(O)O-alkyl, —C(O)OH, —OP(═O)(OR¹¹)₂, —OC(═O)OR¹¹, —OC(═O)R¹¹, cycloalkyl, aryl, heteroaryl or together form an oxo, wherein
each R¹⁵ is independently selected from H, lower alkyl, prenyl, allyl, —C(O)O-alkyl, cycloalkyl, aryl, heteroaryl, alkaryl and alkheteroaryl,
or two of R¹⁵ combine to form an optionally substituted heterocycloalkyl wherein each optionally substituted group is independently selected from R^{b};
R⁷⁵ is straight or branched, saturated or unsaturated alkyl, allyl, cycloalkyl, cycloalkyl, heterocycloalkyl, aryl, heteroaryl, prenylalkaryl, or heteroarylalkyl, each of which is optionally substituted wherein each optionally substituted with one or more R^{b} groups; and
q is an integer from 0 to 10; and
R^{p2} is —C(R^{d}R^{d})_{y}-A-R³, wherein
each R^{d} is independently hydrogen, cyano, —C(O)R^{e1}, —C(O)OR^{e1}, —C(O)NR^{e1}R^{e1}, —C(OR^{e1})(OR^{e1}), optionally substituted (C₁-C₂₀) alkyl, (C₁-C₂₀) perfluoroalkyl, optionally substituted (C₇-C₃₀) arylalkyl, or optionally substituted 6-30 membered heteroarylalkyl, wherein
each R^{e1} is independently hydrogen, alkyl, aryl, arylalkyl, heteroaryl, or heteroarylalkyl;
y is 1, 2, or 3;
A is O, S or NR⁵⁰, wherein R⁵⁰ is R^{d} or cycloalkyl; and
R³ is —R^{f}, —C(O)R^{f}, —C(O)O—R^{f}, —C(O)NR^{f}R^{f}, —Si(R^{f})₃, —P(O)(OH)₂, —P(O)(OH)(OR^{e}), —P(O)(OR^{e})₂, —P(OH)₂, —P(OH)(OR^{e}), or —P(OR^{e})₂, wherein
each R^{e} is independently (i) substituted or unsubstituted lower alkyl, substituted or unsubstituted (C₆-C₁₄) aryl, or substituted or unsubstituted (C₇-C₂₀) arylalkyl wherein each is optionally substituted with one or more groups independently selected from R^{b}, or (ii) —(CR^{d}R^{d})_{y}—OR^{f}, —(CR^{d}R^{d})_{y}—O—C(O)R^{f}, —(CR^{d}R^{d})_{y}—O—C(O)OR^{f}, —(CR^{d}R^{d})_{y}—S—C(O)R^{f}, —(CR^{d}R^{d})_{y}—S—C(O)OR^{f}, —(CR^{d}R^{d})_{y}—NH—C(O)R^{f}, —(CR^{d}R^{d})_{y}—NH—C(O)OR^{f}, or —Si(R^{f})₃;
or two R^{e} taken together with the oxygen atoms to which they are attached, form a 5-8 membered heterocycloalkyl group optionally substituted with substituted or unsubstituted lower alkyl, substituted or unsubstituted lower heterocycloalkyl, substituted or unsubstituted (C₆-C₁₄) aryl, substituted or unsubstituted (C₇-C₂₀) arylalkyl, or substituted or unsubstituted 5-14 membered heteroaryl wherein each is optionally substituted with one or more groups independently selected from R^{b};
each R^{f} group is independently hydrogen, optionally substituted lower alkyl, optionally substituted lower heteroalkyl, optionally substituted lower cycloalkyl, optionally substituted lower heterocycloalkyl, optionally substituted (C₆-C₁₀) aryl, optionally substituted 5-10 membered heteroaryl, optionally substituted (C₇-C₁₈) arylalkyl, or optionally substituted 6-18 membered heteroarylalkyl, wherein each is optionally substituted with one or more groups independently selected from R^{b},
or R⁵⁰ and R³ taken together with nitrogen atom to which they are both attached, form a three- to seven-membered ring.

40. The method of claim 39 wherein the prodrug of a RET kinase inhibitory compound is of the formula,

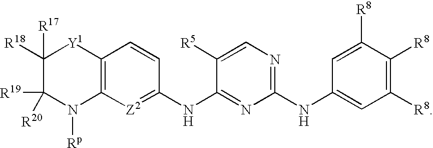

41. The method of claim 40, wherein R⁵ is fluoro.

42. The method of claim 40, wherein each R⁸ is independently hydrogen, hydroxy, or lower alkoxy.

43. The method of claim 40, wherein
R^{p} is —(CR^{d}R^{d})_{y}—O—P(O)(OH)(OH), —(CR^{d}R^{d})_{y}—O—P(O)(OH)(OR^{e}), —(CR^{d}R^{d})_{y}—O—P(O)(OR^{e})₂, —(CR^{d}R^{d})_{y}—O—P(OH)(OR^{e}), or —(CR^{d}R^{d})_{y}—O—P(OR^{e})₂, wherein
y is an integer ranging from 1 to 3;
each R^{e} is independently substituted or unsubstituted lower alkyl, substituted or unsubstituted (C₆-C₁₄) aryl, substituted or unsubstituted (C₇-C₂₀) arylalkyl, —(CR^{d}R^{d})_{y}—OR^{f}, —(CR^{d}R^{d})_{y}—O—C(O)R^{f}, —(CR^{d}R^{d})_{y}—O—C(O)OR^{f}, —(CR^{d}R^{d})_{y}—S—C(O)R^{f}, —(CR^{d}R^{d})_{y}—S—C(O)OR^{f}, —(CR^{d}R^{d})_{y}—NHC(O)R^{f}, —(CR^{d}R^{d})_{y}—NHC(O)OR^{f}, or —Si(R^{f})₃ wherein
each R^{f} is independently hydrogen, substituted or unsubstituted lower alkyl, substituted or unsubstituted (C₆-C₁₄) aryl, or substituted or unsubstituted (C₇-C₂₀) arylalkyl.

44. The method of claim 43, wherein
R^{p} is —(CH₂)_{y}—O—P(O)(OH)(OH), —(CH₂)_{y}—O—P(O)(OH)(OR^{e}), —(CH₂)_{y}—O—P(O)(OR^{e})₂, —(CH₂)_{y}—O—P(OH)(OR^{e}), or —(CH₂)_{y}—O—P(OR^{e})₂, wherein
y is 1, 2, or 3;
each R^{e} is independently substituted or unsubstituted lower alkyl, substituted or unsubstituted (C₆-C₁₄) aryl, substituted or unsubstituted (C₇-C₂₀) arylalkyl.

45. The method of claim 44, wherein R^{p} is —CH₂—O—P(O)(OH)(OH).

46. The method of claim 39, wherein the prodrug of a RET kinase inhibitory compound is of the formula, 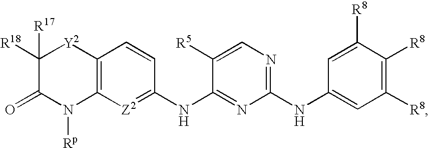
wherein Y² is O, S, S(O) or S(O)₂.

47. The method of claim 46, wherein R⁵ is fluoro.

48. The method of claim 46 wherein each R⁸ is independently hydrogen, hydroxy, or lower alkoxy.

49. The method of claim 46, wherein
R^{p} is —(CR^{d}R^{d})_{y}—O—P(O)(OH)(OH), —(CR^{d}R^{d})_{y}—O—P(O)(OH)(OR^{e}), —(CR^{d}R^{d})_{y}—O—P(O)(OR^{e})₂, —(CR^{d}R^{d})_{y}—O—P(OH)(OR^{e}), or —(CR^{d}R^{d})_{y}—O—P(OR^{e})₂, wherein
y is 1, 2, or 3;
each R^{e} is independently substituted or unsubstituted lower alkyl, substituted or unsubstituted (C₆-C₁₄) aryl, substituted or unsubstituted (C₇-C₂₀) arylalkyl, —(CR^{d}R^{d})_{y}—OR^{f}, —(CR^{d}R^{d})_{y}—O—C(O)R^{f}, —(CR^{d}R^{d})_{y}—O—C(O)OR^{f}, —(CR^{d}R^{d})_{y}—S—C(O)R^{f}, —(CR^{d}R^{d})_{y}—S—C(O)OR^{f}, —(CR^{d}R^{d})_{y}—NHC(O)R^{f}, —(CR^{d}R^{d})_{y}—NHC(O)OR^{f}, or —Si(R^{f})₃, wherein
each R^{f} is independently hydrogen, substituted or unsubstituted lower alkyl, substituted or unsubstituted (C₆-C₁₄) aryl, or substituted or unsubstituted (C₇-C₂₀) arylalkyl.

50. The method of claim 49, wherein
R^{p} is —(CH₂)_{y}—O—P(O)(OH)(OH), —(CH₂)_{y}—O—P(O)(OH)(OR^{e}), —(CH₂)_{y}—O—P(O)(OR^{e})₂, —(CH₂)_{y}—O—P(OH)(OR^{e}), or —(CH₂)_{y}—O—P(OR^{e})₂, wherein
y is 1, 2, or 3;
each R^{e} is independently substituted or unsubstituted lower alkyl, substituted or unsubstituted (C₆-C₁₄) aryl, substituted or unsubstituted (C₇-C₂₀) arylalkyl.

51. The method of claim 50, wherein R^{p} is —CH₂—O—P(O)(OH)(OH).

52. The method of claim 39 wherein the compound is administered in the form of a pharmaceutical composition.

53. The method of claim 39 wherein the compound is administered orally or intravenously.

54. The method of claim 39, wherein the compound is
6-(5-fluoro-2-(3,4,5-trimethoxyphenylamino)pyrimidin-4-ylamino)-2,2-dimethyl-2H-pyrido[3,2-b][1,4]oxazin-3(4H)-one;
2-(3-(4-(2,2-difluoro-3-oxo-3,4-dihydro-2H-pyrido[3,2-b][1,4]oxazin-6-ylamino)-5-fluoropyrimidin-2-ylamino)phenoxy)-N-methylacetamide;
6-(5-fluoro-2-(2-methyl-1H-benzo[d]imidazol-6-ylamino)pyrimidin-4-ylamino)-2,2-dimethyl-2H-pyrido[3,2-b][1,4]thiazin-3(4H)-one;
6-(5-fluoro-2-(3-hydroxy-4,5-dimethoxyphenylamino)pyrimidin-4-ylamino)-2,2-dimethyl-2H-pyrido[3,2-b][1,4]oxazin-3(4H)-one;
or a pharmaceutically acceptable salt, solvate, hydrate, or N-oxide thereof.

55. A method of inhibiting proliferation of a tumor cell, comprising, administering to a tumor cell an amount of a drug compound according to structural formula (I) effective to inhibit proliferation of the tumor cell: 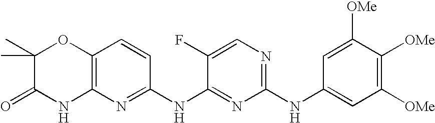
or a pharmaceutically acceptable salt, hydrate, solvate or N-oxide thereof.

56. The method of claim 55, which is carried out in vitro.

57. The method of claim 55, which is carried out in vivo in a subject.

58. The method of claim 55, in which the tumor cell is a renal tumor cell.

59. The method of claim 55, in which the tumor cell is a thyroid tumor cell.

60. A method of inhibiting the proliferation of a tumor cell comprising administering to a tumor cell a prodrug compound according to the structural formula, 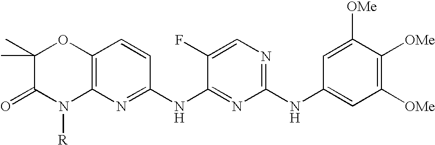
or a pharmaceutically acceptable salt, hydrate, solvate or N-oxide thereof, wherein
R^{p1} is R^{p1} or R^{p2}, wherein
R^{p1} is —C(═X²)—X¹—(CR⁵⁵R⁶⁵)_{q}—R⁷⁵, wherein
X¹ is O, S, or NR¹¹, wherein each R¹¹ is independently H or lower alkyl;
X² is O or S;
R⁵⁵ and R⁶⁵ are each independently H, OH, —OR¹¹, NR¹⁵R¹⁵, halo, lower alkyl, —C(O)O-alkyl, —C(O)OH, —OP(═O)(OR¹¹)₂, —OC(═O)OR¹¹, —OC(═O)R¹¹, cycloalkyl, aryl, heteroaryl or together form an oxo, wherein
each R¹⁵ is independently selected from H, lower alkyl, prenyl, allyl, —C(O)O-alkyl, cycloalkyl, aryl, heteroaryl, alkaryl and alkheteroaryl,
or two of R¹⁵ combine to form an optionally substituted heterocycloalkyl wherein each optionally substituted group is independently selected from R^{b};
R⁷⁵ is straight or branched, saturated or unsaturated alkyl, allyl, cycloalkyl, cycloalkyl, heterocycloalkyl, aryl, heteroaryl, prenylalkaryl, or heteroarylalkyl, each of which is optionally substituted wherein each optionally substituted with one or more R¹ groups; and
q is an integer from 0 to 10; and
R^{p2} is —C(R^{d}R^{d})_{y}-A-R³, wherein
each R^{d} is independently hydrogen, cyano, —C(O)R^{e1}, —C(O)OR^{e1}, —C(O)NR^{e1}R^{e1}, —C(OR^{e1})(OR^{e1}), optionally substituted (C₁-C₂₀) alkyl, (C₁-C₂₀) perfluoroalkyl, optionally substituted (C₇-C₃₀) arylalkyl, or optionally substituted 6-30 membered heteroarylalkyl, wherein
each R^{e1} is independently hydrogen, alkyl, aryl, arylalkyl, heteroaryl, or heteroarylalkyl;
y is 1, 2, or 3;
A is O, S or NR⁵⁰, wherein R⁵⁰ is R^{d} or cycloalkyl; and
R³ is —R^{f}, —C(O)R^{f}, —C(O)O—R^{f}, —C(O)NR^{f}R^{f}, —Si(R^{f})₃, —P(O)(OH)₂, —P(O)(OH)(OR^{e}), —P(O)(OR^{e})₂, —P(OH)₂, —P(OH)(OR^{e}), or —P(OR^{e})₂, wherein
each R^{e} is independently (i) substituted or unsubstituted lower alkyl, substituted or unsubstituted (C₆-C₁₄) aryl, or substituted or unsubstituted (C₇-C₂₀) arylalkyl wherein each is optionally substituted with one or more groups independently selected from R^{b}, or (ii)-(CR^{d}R^{d})_{y}—OR^{f}, —(CR^{d}R^{d})_{y}—O—C(O)R^{f}, —(CR^{d}R^{d})_{y}—O—C(O)OR^{f}, —(CR^{d}R^{d})_{y}—S—C(O)R^{f}, —(CR^{d}R^{d})_{y}—S—C(O)OR^{f}, —(CR^{d}R^{d})_{y}—NH—C(O)R^{f}, —(CR^{d}R^{d})_{y}—NH—C(O)OR^{f}, or —Si(R^{f})₃;
or two R^{e} taken together with the oxygen atoms to which they are attached, form a 5-8 membered heterocycloalkyl group optionally substituted with substituted or unsubstituted lower alkyl, substituted or unsubstituted lower heterocycloalkyl, substituted or unsubstituted (C₆-C₁₄) aryl, substituted or unsubstituted (C₇-C₂₀) arylalkyl, or substituted or unsubstituted 5-14 membered heteroaryl wherein each is optionally substituted with one or more groups independently selected from R^{b};
each R^{f} group is independently hydrogen, optionally substituted lower alkyl, optionally substituted lower heteroalkyl, optionally substituted lower cycloalkyl, optionally substituted lower heterocycloalkyl, optionally substituted (C₆-C₁₀) aryl, optionally substituted 5-10 membered heteroaryl, optionally substituted (C₇-C₁₈) arylalkyl, or optionally substituted 6-18 membered heteroarylalkyl, wherein each is optionally substituted with one or more groups independently selected from R^{b},
or R⁵⁰ and R³ taken together with nitrogen atom to which they are both attached, form a three- to seven-membered ring;
each R^{a} is independently hydrogen, lower alkyl, lower cycloalkyl, cyclohexyl, (C₄-C₁₁) cycloalkylalkyl, (C₆-C₁₀) aryl, phenyl, (C₇-C₁₆) arylalkyl, benzyl, 2-6 membered heteroalkyl, 3-8 membered heterocycloalkyl, morpholinyl, piperazinyl, homopiperazinyl, piperidinyl, 4-11 membered heterocycloalkylalkyl, 5-10 membered heteroaryl, or 6-16 membered heteroarylalkyl;
each R^{b} is independently ═O, —OR^{a}, (C₁-C₃) haloalkyloxy, ═S, —SR^{a}, ═NR^{a}, ═NOR^{a}, —NR^{c}R^{c}, halogen, —CF₃, —CN, —NC, —OCN, —SCN, —NO, —NO₂, ═N₂, —N₃, —S(O)R^{a}, —S(O)₂R^{a}, —S(O)₂OR^{a}, —S(O)NR^{c}R^{c}, —S(O)₂NR^{c}R^{c}, —OS(O)₂R^{a}, —OS(O)₂OR^{a}, —OS(O)₂NR^{c}R^{c}, —C(O)R^{a}, —C(O)OR^{a}, —C(O)NR^{c}R^{c}, —C(NH)NR^{c}R^{c} —C(NR^{a})NR^{c}R^{c}, —C(NOH)R^{a}, —C(NOH)NR^{c}R^{c}, —OC(O)R^{a}, —OC(O)OR^{a}, OC(O)NR^{c}R^{c}, —OC(NH)NR^{c}R^{c}, —OC(NR^{a})NR^{c}R^{c}, —[NHC(O)]ₙR^{a}, —[NR^{a}C(O)]ₙR^{a}, —[NHC(O)]ₙOR^{a}, —[NR^{a}C(O)]ₙOR^{a}, —[NHC(O)]ₙNR^{c}R^{c}, —[NR^{a}C(O)]ₙNR^{c}R^{c}, —[NHC(NH)]ₙNR^{c}R^{c}, —[NR^{a}C(NR^{a})]ₙNR^{c}R^{c}; and
each R^{e} is independently R^{a},
or two R^{e} bonded to the same nitrogen atom taken together with the nitrogen atom to which they are both attached form a 5 to 8-membered heterocycloalkyl or heteroaryl group comprising one or more of the same or different additional heteroatoms and optionally substituted with one to four of the same or different R^{a} groups;
in an amount effective to, and under conditions suitable to, yield an amount of a drug compound effective to inhibit proliferation of the tumor cell.

61. The method of claim 60, wherein R^{p} is —(CR^{d}R^{d})_{y}—O—P(O)(OH)(OH),
—(CR^{d}R^{d})_{y}—O—P(O)(OH)(OR^{e}), —(CR^{d}R^{d})_{y}—O—P(O)(OR^{e})₂, —(CR^{d}R^{d})_{y}—O—P(OH)(OR^{e}), or —(CR^{d}R^{d})_{y}—O—P(OR^{e})₂, wherein
y is 1, 2, or 3;
each R^{e} is independently substituted or unsubstituted lower alkyl, substituted or unsubstituted (C₆-C₁₄) aryl, substituted or unsubstituted (C₇-C₂₀) arylalkyl, —(CR^{d}R^{d})_{y}—OR, —(CR^{d}R^{d})_{y}—O—C(O)R^{f}, —(CR^{d}R^{d})_{y}—O—C(O)OR^{f}, —(CR^{d}R^{d})_{y}—S—C(O)R^{f}, (CR^{d}R^{d})_{y}—S—C(O)OR^{f}, (CR^{d}R^{d})_{y}—NH—C(O)R^{f}, —(CR^{d}R^{d})_{y}—NH—C(O)OR^{f}, or —Si(R^{f})₃, wherein
each R^{f} is independently hydrogen, substituted or unsubstituted lower alkyl, substituted or unsubstituted (C₆-C₁₄) aryl, or substituted or unsubstituted (C₇-C₂₀) arylalkyl.

62. The method of claim 61, in which R is —CH₂—O—P(O)(OH)₂, or and ionized form or salt thereof.

63. The method of claim 60, which is carried out in vitro.

64. The method of claim 60, which is carried out in vivo in a subject.

65. The method of claim 60, in which the tumor cell is a renal tumor cell.

66. The method of claim 60, in which the tumor cell is a thyroid tumor cell.

67. A method of treating a solid tumor cancer in a subject, comprising administering to a subject an amount of a compound according to the structural formula, 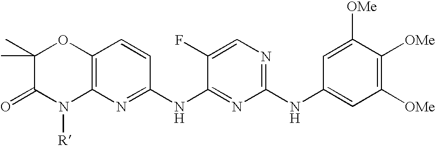
or a pharmaceutically acceptable salt, hydrate, solvate or N-oxide thereof, wherein R′ is selected from hydrogen and R^{p}, wherein R^{p} is R^{p1} or R^{p2}, wherein
R^{p1} is —C(═X²)—X¹—(CR⁵⁵R⁶⁵)_{q}—R⁷⁵, wherein
X¹ is O, S, or NR¹¹, wherein each R¹¹ is independently H or lower alkyl;
X² is O or S;
R⁵⁵ and R⁶⁵ are each independently H, OH, —OR¹¹, NR¹⁵R¹⁵, halo, lower alkyl, —C(O)O-alkyl, —C(O)OH, —OP(═O)(OR¹¹)₂, —OC(═O)OR¹¹, —OC(═O)R¹¹, cycloalkyl, aryl, heteroaryl or together form an oxo, wherein
each R¹⁵ is independently selected from H, lower alkyl, prenyl, allyl, —C(O)O-alkyl, cycloalkyl, aryl, heteroaryl, alkaryl and alkheteroaryl,
or two of R¹⁵ combine to form an optionally substituted heterocycloalkyl wherein each optionally substituted group is independently selected from R^{b};
R⁷⁵ is straight or branched, saturated or unsaturated alkyl, allyl, cycloalkyl, cycloalkyl, heterocycloalkyl, aryl, heteroaryl, prenylalkaryl, or heteroarylalkyl, each of which is optionally substituted wherein each optionally substituted with one or more R^{b} groups; and
q is an integer from 0 to 10; and
R^{p2} is —C(R^{d}R^{d})_{y}-A-R³, wherein
each R^{d} is independently hydrogen, cyano, —C(O)R^{e1}, —C(O)OR^{e1}, —C(O)NR^{e1}R^{e1}, —C(OR^{e1})(OR^{e1}), optionally substituted (C₁-C₂₀) alkyl, (C₁-C₂₀) perfluoroalkyl, optionally substituted (C₇-C₃₀) arylalkyl, or optionally substituted 6-30 membered heteroarylalkyl, wherein
each R^{e1} is independently hydrogen, alkyl, aryl, arylalkyl, heteroaryl, or heteroarylalkyl;
y is 1, 2, or 3;
A is O, S or NR⁵⁰, wherein R⁵⁰ is R^{d} or cycloalkyl; and
R³ is —R^{f}, —C(O)R^{f}, —C(O)O—R^{f}, —C(O)NR^{f}R^{f}, —Si(R^{f})₃, —P(O)(OH)₂, —P(O)(OH)(OR^{e}), —P(O)(OR^{e})₂, —P(OH)₂, —P(OH)(OR^{e}), or —P(OR^{e})₂, wherein
each R^{e} is independently (i) substituted or unsubstituted lower alkyl, substituted or unsubstituted (C₆-C₁₄) aryl, or substituted or unsubstituted (C₇-C₂₀) arylalkyl wherein each is optionally substituted with one or more groups independently selected from R^{b}, or (ii) —(CR^{d}R^{d})_{y}—OR^{f}, —(CR^{d}R^{d})_{y}—O—C(O)R^{f}, —(CR^{d}R^{d})_{y}—O—C(O)OR^{f}, —(CR^{d}R^{d})_{y}—S—C(O)R^{f}, —(CR^{d}R^{d})_{y}—S—C(O)OR^{f}, —(CR^{d}R^{d})_{y}—NH—C(O)R^{f}, —(CR^{d}R^{d})_{y}—NH—C(O)OR^{f}, or —Si(R^{f})₃;
or two R^{e} taken together with the oxygen atoms to which they are attached, form a 5-8 membered heterocycloalkyl group optionally substituted with substituted or unsubstituted lower alkyl, substituted or unsubstituted lower heterocycloalkyl, substituted or unsubstituted (C₆-C₁₄) aryl, substituted or unsubstituted (C₇-C₂₀) arylalkyl, or substituted or unsubstituted 5-14 membered heteroaryl wherein each is optionally substituted with one or more groups independently selected from R^{b};
each R^{f} group is independently hydrogen, optionally substituted lower alkyl, optionally substituted lower heteroalkyl, optionally substituted lower cycloalkyl, optionally substituted lower heterocycloalkyl, optionally substituted (C₆-C₁₀) aryl, optionally substituted 5-10 membered heteroaryl, optionally substituted (C₇-C₁₈) arylalkyl, or optionally substituted 6-18 membered heteroarylalkyl, wherein each is optionally substituted with one or more groups independently selected from R^{b},
or R⁵⁰ and R³ taken together with nitrogen atom to which they are both attached, form a three- to seven-membered ring;
each R^{a} is independently hydrogen, lower alkyl, lower cycloalkyl, cyclohexyl, (C₄-C₁₁) cycloalkylalkyl, (C₆-C₁₀) aryl, phenyl, (C₇-C₁₆) arylalkyl, benzyl, 2-6 membered heteroalkyl, 3-8 membered heterocycloalkyl, morpholinyl, piperazinyl, homopiperazinyl, piperidinyl, 4-11 membered heterocycloalkylalkyl, 5-10 membered heteroaryl, or 6-16 membered heteroarylalkyl;
each R^{b} is independently ═O, —OR^{a}, (C₁-C₃) haloalkyloxy, ═S, —SR^{a}, ═NR^{a}, ═NOR^{a}, —NR^{c}R^{c}, halogen, —CF₃, —CN, —NC, —OCN, —SCN, —NO, —NO₂, ═N₂, —N₃, —S(O)R^{a}, —S(O)₂R^{a}, —S(O)₂OR^{a}, —S(O)NR^{c}R^{c}, —S(O)₂NR^{c}R^{c}, —OS(O)₂R^{a}, —OS(O)₂OR^{a}, —OS(O)₂NR^{c}R^{c}, —C(O)R^{a}, —C(O)OR^{a}, —C(O)NR^{c}R^{c}, —C(NH)NR^{c}R^{c} —C(NR^{a})NR^{c}R^{c}, —C(NOH)R^{a}, —C(NOH)NR^{c}R^{c}, —OC(O)R^{a}, —OC(O)OR^{a}, OC(O)NR^{c}R^{c}, —OC(NH)NR^{c}R^{c}, —OC(NR^{a})NR^{c}R^{c}, —[NHC(O)]ₙR^{a}, —[NR^{a}C(O)]ₙR^{a}, —[NHC(O)]ₙOR^{a}, —[NR^{a}C(O)]ₙOR^{a}, —[NHC(O)]ₙNR^{c}R^{c}, —[NR^{a}C(O)]ₙNR^{c}R^{c}, —[NHC(NH)]ₙNR^{c}R^{c}, —[NR^{a}C(NR^{a})]ₙNR^{c}R^{c}; and
each R^{e} is independently R^{a},
or two R^{e} bonded to the same nitrogen atom taken together with the nitrogen atom to which they are both attached form a 5 to 8-membered heterocycloalkyl or heteroaryl group comprising one or more of the same or different additional heteroatoms and optionally substituted with one to four of the same or different R^{a} groups;
effective to treat the solid tumor cancer.

68. The method of claim 67, in which R′ is hydrogen.

69. The method of claim 67, in which R′ is a R^{p}.

70. The method of claim 69, wherein R^{p} is —(CR^{d}R^{d})_{y}—O—P(O)(OH)(OH), —(CR^{d}R^{d})_{y}—O—P(O)(OH)(OR^{e}), —(CR^{d}R^{d})_{y}—O—P(O)(OR^{e})₂, —(CR^{d}R^{d})_{y}—O—P(OH)(OR^{e}), or —(CR^{d}R^{d})_{y}—O—P(OR^{e})₂, wherein
y is 1, 2, or 3;
each R^{e} is independently substituted or unsubstituted lower alkyl, substituted or unsubstituted (C₆-C₁₄) aryl, substituted or unsubstituted (C₇-C₂₀) arylalkyl, —(CR^{d}R^{d})_{y}—OR, —(CR^{d}R^{d})_{y}—O—C(O)R^{f}, —(CR^{d}R^{d})_{y}—O—C(O)OR^{f}, —(CR^{d}R^{d})_{y}—S—C(O)R^{f}, (CR^{d}R^{d})_{y}—S—C(O)OR^{f}, (CR^{d}R^{d})_{y}—NH—C(O)R^{f}, —(CR^{d}R^{d})_{y}—NH—C(O)OR^{f}, or —Si(R^{f})₃, wherein
each R^{f} is independently hydrogen, substituted or unsubstituted lower alkyl, substituted or unsubstituted (C₆-C₁₄) aryl, or substituted or unsubstituted (C₇-C₂₀) arylalkyl;

71. The method of claim 70, in which R^{p} is —CH₂—O—P(O)(OH)₂, or an ionized form or salt thereof.

72. The method of claim 67, in which the compound is administered in the form of a pharmaceutical composition.

73. The method of claim 67, in which the compound is administered orally or intravenously.

74. The method of claim 67, in which the solid tumor cancer is selected from renal cell carcinoma, ovarian carcinoma, kidney carcinoma, clear cell carcinoma of kidney, renal cell adenocarcinoma, ovarian adenocarcinoma, colon adenocarcinoma, lung adenocarcinoma, large cell lung carcinoma, squamous cell carcinoma of the lung, mesothelioma, and glioma.

75. The method of claim 74, in which the solid tumor cancer is renal cell carcinoma and/or renal cell adenocarcinoma.

76. The method of claim 67, in which the subject is a human.

77. The method of claim 67, in which the solid tumor cancer is medullary thyroid carcinomas, papillary thyroid carcinomas, multiple endocrine neoplasia type 2A (MEN2A), parathyroid adenomas, multiple endocrine neoplasia type 2B (MEN2B), or familial medullary thyroid carcinoma (FMTC).

78. The method of claim 77, in which the solid tumor cancer is medullary thyroid carcinoma.

79. A method for treating a disease or condition caused by a mutation in RET kinase, comprising administering to a subject in need of such treatment an amount of a compound according to the structural formula, 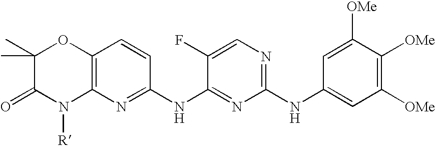
or a pharmaceutically acceptable salt, hydrate, solvate or N-oxide thereof, wherein R′ is selected from hydrogen and a R^{p}, wherein R^{p} is R^{p1} or R^{p2}, wherein
R^{p1} is —C(═X²)—X¹—(CR⁵⁵R⁶⁵)_{q}—R⁷⁵, wherein
X¹ is O, S, or NR¹¹, wherein each R¹¹ is independently H or lower alkyl;
X² is O or S;
R⁵⁵ and R⁶⁵ are each independently H, OH, —OR¹¹, NR¹⁵R¹⁵, halo, lower alkyl, —C(O)O-alkyl, —C(O)OH, —OP(═O)(OR¹¹)₂, —OC(═O)OR¹¹, —OC(═O)R¹¹, cycloalkyl, aryl, heteroaryl or together form an oxo, wherein
each R¹⁵ is independently selected from H, lower alkyl, prenyl, allyl, —C(O)O-alkyl, cycloalkyl, aryl, heteroaryl, alkaryl and alkheteroaryl,
or two of R¹⁵ combine to form an optionally substituted heterocycloalkyl wherein each optionally substituted group is independently selected from R^{b};
R⁷⁵ is straight or branched, saturated or unsaturated alkyl, allyl, cycloalkyl, cycloalkyl, heterocycloalkyl, aryl, heteroaryl, prenylalkaryl, or heteroarylalkyl, each of which is optionally substituted wherein each optionally substituted with one or more R^{b} groups; and
q is an integer from 0 to 10; and
R^{p2} is —C(R^{d}R^{d})_{y}-A-R³, wherein
each R^{d} is independently hydrogen, cyano, —C(O)R^{e1}, —C(O)OR^{e1}, —C(O)NR^{e1}R^{e1}, —C(OR^{e1})(OR^{e1}), optionally substituted (C₁-C₂₀) alkyl, (C₁-C₂₀) perfluoroalkyl, optionally substituted (C₇-C₃₀) arylalkyl, or optionally substituted 6-30 membered heteroarylalkyl, wherein
each R^{e1} is independently hydrogen, alkyl, aryl, arylalkyl, heteroaryl, or heteroarylalkyl;
y is 1, 2, or 3;
A is O, S or NR⁵⁰, wherein R⁵⁰ is R^{d} or cycloalkyl; and
R³ is —R^{f}, —C(O)R^{f}, —C(O)O—R^{f}, —C(O)NR^{f}R^{f}, —Si(R^{f})₃, —P(O)(OH)₂, —P(O)(OH)(OR^{e}), —P(O)(OR^{e})₂, —P(OH)₂, —P(OH)(OR^{e}), or —P(OR^{e})₂, wherein
each R^{e} is independently (i) substituted or unsubstituted lower alkyl, substituted or unsubstituted (C₆-C₁₄) aryl, or substituted or unsubstituted (C₇-C₂₀) arylalkyl wherein each is optionally substituted with one or more groups independently selected from R^{b}, or (ii)-(CR^{d}R^{d})_{y}—OR^{f}, —(CR^{d}R^{d})_{y}—O—C(O)R^{f}, —(CR^{d}R^{d})_{y}—O—C(O)OR^{f}, —(CR^{d}R^{d})_{y}—S—C(O)R^{f}, —(CR^{d}R^{d})_{y}—S—C(O)OR^{f}, —(CR^{d}R^{d})_{y}—NH—C(O)R^{f}, —(CR^{d}R^{d})_{y}—NH—C(O)OR^{f}, or —Si(R^{f})₃;
or two R^{e} taken together with the oxygen atoms to which they are attached, form a 5-8 membered heterocycloalkyl group optionally substituted with substituted or unsubstituted lower alkyl, substituted or unsubstituted lower heterocycloalkyl, substituted or unsubstituted (C₆-C₁₄) aryl, substituted or unsubstituted (C₇-C₂₀) arylalkyl, or substituted or unsubstituted 5-14 membered heteroaryl wherein each is optionally substituted with one or more groups independently selected from R^{b};
each R^{f} group is independently hydrogen, optionally substituted lower alkyl, optionally substituted lower heteroalkyl, optionally substituted lower cycloalkyl, optionally substituted lower heterocycloalkyl, optionally substituted (C₆-C₁₀) aryl, optionally substituted 5-10 membered heteroaryl, optionally substituted (C₇-C₁₈) arylalkyl, or optionally substituted 6-18 membered heteroarylalkyl, wherein each is optionally substituted with one or more groups independently selected from R^{b},
or R⁵⁰ and R³ taken together with nitrogen atom to which they are both attached, form a three- to seven-membered ring;
each R^{a} is independently hydrogen, lower alkyl, lower cycloalkyl, cyclohexyl, (C₄-C₁₁) cycloalkylalkyl, (C₆-C₁₀) aryl, phenyl, (C₇-C₁₆) arylalkyl, benzyl, 2-6 membered heteroalkyl, 3-8 membered heterocycloalkyl, morpholinyl, piperazinyl, homopiperazinyl, piperidinyl, 4-11 membered heterocycloalkylalkyl, 5-10 membered heteroaryl, or 6-16 membered heteroarylalkyl;
each R^{b} is independently ═O, —OR^{a}, (C₁-C₃) haloalkyloxy, ═S, —SR^{a}, ═NR^{a}, ═NOR^{a}, —NR^{c}R^{c}, halogen, —CF₃, —CN, —NC, —OCN, —SCN, —NO, —NO₂, ═N₂, —N₃, —S(O)R^{a}, —S(O)₂R^{a}, —S(O)₂OR^{a}, —S(O)NR^{c}R^{c}, —S(O)₂NR^{c}R^{c}, —OS(O)₂R^{a}, —OS(O)₂OR^{a}, —OS(O)₂NR^{c}R^{c}, —C(O)R^{a}, —C(O)OR^{a}, —C(O)NR^{c}R^{c}, —C(NH)NR^{c}R^{c} —C(NR^{a})NR^{c}R^{c}, —C(NOH)R^{a}, —C(NOH)NR^{c}R^{c}, —OC(O)R^{a}, —OC(O)OR^{a}, OC(O)NR^{c}R^{c}, —OC(NH)NR^{c}R^{c}, —OC(NR^{a})NR^{c}R^{c}, —[NHC(O)]ₙR^{a}, —[NR^{a}C(O)]ₙR^{a}, —[NHC(O)]ₙOR^{a}, —[NR^{a}C(O)]ₙOR^{a}, —[NHC(O)]ₙNR^{c}R^{c}, —[NR^{a}C(O)]ₙNR^{c}R^{c}, —[NHC(NH)]ₙNR^{c}R^{c}, —[NR^{a}C(NR^{a})]ₙNR^{c}R^{c}; and
each R^{e} is independently R^{a},
or two R^{e} bonded to the same nitrogen atom taken together with the nitrogen atom to which they are both attached form a 5 to 8-membered heterocycloalkyl or heteroaryl group comprising one or more of the same or different additional heteroatoms and optionally substituted with one to four of the same or different R^{a} groups;
effective to inhibit at least one activity of the mutated RET kinase.

80. The method of claim 79, in which R′ is hydrogen.

81. The method of claim 79, in which R′ is a R^{p}.

82. The method of claim 81, in which R^{p} is —(CR^{d}R^{d})_{y}—O—P(O)(OH)(OH), —(CR^{d}R^{d})_{y}—O—P(O)(OH)(OR^{e}), —(CR^{d}R^{d})_{y}—O—P(O)(OR^{e})₂, —(CR^{d}R^{d})_{y}—O—P(OH)(OR^{e}), or —(CR^{d}R^{d})_{y}—O—P(OR^{e})₂, wherein
y is 1, 2, or 3;
each R^{e} is independently substituted or unsubstituted lower alkyl, substituted or unsubstituted (C₆-C₁₄) aryl, substituted or unsubstituted (C₇-C₂₀) arylalkyl, —(CR^{d}R^{d})_{y}—OR^{f}, —(CR^{d}R^{d})_{y}—O—C(O)R^{f}, —(CR^{d}R^{d})_{y}—O—C(O)OR^{f}, —(CR^{d}R^{d})_{y}—S—C(O)R^{f}, (CR^{d}R^{d})_{y}—S—C(O)OR^{f}, (CR^{d}R^{d})_{y}—NH—C(O)R^{f}, —(CR^{d}R^{d})_{y}—NH—C(O)OR^{f}, or —Si(R^{f})₃, wherein
each R^{f} is independently hydrogen, substituted or unsubstituted lower alkyl, substituted or unsubstituted (C₆-C₁₄) aryl, or substituted or unsubstituted (C₇-C₂₀) arylalkyl.

83. The method of claim 82, in which R^{p} is —CH₂—O—P(O)(OH)₂, or an ionized form or salt thereof.

84. The method of claim 79, in which the compound is administered in the form of a pharmaceutical composition.

85. The method of claim 79, in which the compound is administered orally or intravenously.

86. The method of claim 79, in which the disease or condition is a thyroid cancer.

87. The method of claim 79, in which the disease or condition is medullary thyroid carcinomas, papillary thyroid carcinomas, multiple endocrine neoplasia type 2A (MEN2A), parathyroid adenomas, multiple endocrine neoplasia type 2B (MEN2B), or familial medullary thyroid carcinoma (FMTC).

88. The method of claim 87, in which the disease or condition is medullary thyroid carcinoma.
